(19) United States
(12) Patent Application Publication
(10) Pub. No.: US 2013/0022649 A1
(43) Pub. Date: Jan. 24, 2013

(54) SNALP FORMULATIONS CONTAINING ANTIOXIDANTS

(71) Applicants: Ed Yaworski, Maple Ridge (CA); James Heyes, Vancouver (CA); Adam Judge, Vancouver (CA); Stephen Reid, Surrey (CA); Ian MacLachlan, Mission (CA)

(72) Inventors: Ed Yaworski, Maple Ridge (CA); James Heyes, Vancouver (CA); Adam Judge, Vancouver (CA); Stephen Reid, Surrey (CA); Ian MacLachlan, Mission (CA)

(73) Assignee: Protiva Biotherapeutics, Inc., Burnaby, BC (CA)

(21) Appl. No.: 13/513,548

(22) PCT Filed: Dec. 1, 2010
(86) PCT No.: PCT/CA2010/001919

§ 371 Date: Sep. 19, 2012

Related U.S. Application Data
(60) Provisional application No. 61/265,671, filed on Dec. 1, 2009.

(51) Int. Cl. A61K 31/7088; A61K 9/14; B82Y 5/00

(52) U.S. Cl. 424/400; 514/44 R; 977/773

(57) ABSTRACT

The present invention provides methods of preventing, decreasing, or inhibiting the degradation of cationic lipids and/or active agents (e.g., therapeutic nucleic acids) present in lipid particles, compositions comprising lipid particles stabilized by these methods, methods of making these lipid particles, and methods of delivering and/or administering these lipid particles, e.g., for the treatment of a disease or disorder.

### CROSS-REFERENCE TO RELATED APPLICATION

The present application claims priority to U.S. Provisional Application No. 61/265,671, filed Dec. 1, 2009, the disclosure of which is hereby incorporated by reference in its entirety for all purposes.

### BACKGROUND OF THE INVENTION

Therapeutic nucleic acids include, e.g., small interfering RNA (siRNA), microRNA (miRNA), antisense oligonucleotides, ribozymes, plasmids, and immune-stimulating nucleic acids. These nucleic acids act via a variety of mechanisms. In the case of interfering RNA molecules such as siRNA and miRNA, these nucleic acids can down-regulate intracellular levels of specific proteins through a process termed RNA interference (RNAi). Following introduction of interfering RNA into the cell cytoplasm, these double-stranded RNA constructs can bind to a protein termed RISC. The sense strand of the interfering RNA is displaced from the RISC complex, providing a template within RISC that can recognize and bind mRNA with a complementary sequence to that of the bound interfering RNA. Having bound the complementary mRNA, the RISC complex cleaves the mRNA and releases the cleaved strands. RNAi can provide down-regulation of specific proteins by targeting specific destruction of the corresponding mRNA that encodes for protein synthesis.

The therapeutic applications of RNAi are extremely broad, since interfering RNA constructs can be synthesized with any nucleotide sequence directed against a target protein. To date, siRNA constructs have shown the ability to specifically down-regulate target proteins in both in vitro and in vivo models. In addition, siRNA constructs are currently being evaluated in clinical studies.

However, two problems currently faced by interfering RNA constructs are, first, their susceptibility to nuclease digestion in plasma and, second, their limited ability to gain access to the intracellular compartment where they can bind RISC when administered systemically as free interfering RNA molecules. These double-stranded constructs can be stabilized by the incorporation of chemically modified nucleotide linkers within the molecule, e.g., phosphothioate groups. However, such chemically modified linkers provide only limited protection from nuclease digestion and may decrease the activity of the construct. Intracellular delivery of interfering RNA can be facilitated by the use of carrier systems such as polymers, cationic liposomes, or by the covalent attachment of a cholesterol moiety to the molecule. However, improved delivery systems are required to increase the potency of interfering RNA molecules such as siRNA and miRNA and to reduce or eliminate the requirement for chemically modified nucleotide linkers.

In addition, problems remain with the limited ability of therapeutic nucleic acids such as interfering RNA to cross cellular membranes (see, Vlassov et al., Biochim. Biophys. Acta, 1197:95-1082 (1994)) and in the problems associated with systemic toxicity, such as complement-mediated anaphylaxis, altered coagulatory properties, and cytopenia (Galbraith et al., Antisense Nucl. Acid Drug Des., 4:201-206 (1994)).

To attempt to improve efficacy, investigators have also employed lipid-based carrier systems to deliver chemically modified or unmodified therapeutic nucleic acids. Zelphati et al. (J. Contr. Rel., 41:99-119 (1996)) describes the use of anionic (conventional) liposomes, pH sensitive liposomes, immunoliposomes, fusogenic liposomes, and cationic lipid/antisense aggregates. Similarly, siRNA has been administered systemically in cationic liposomes, and these nucleic acid-lipid particles have been reported to provide improved down-regulation of target proteins in mammals including non-human primates (Zimmermann et al., Nature, 441: 111-114 (2006)).

In spite of this progress, there remains a need in the art for improved lipid-therapeutic nucleic acid compositions that are suitable for general therapeutic use. Preferably, these compositions encapsulate nucleic acids with high-efficiency, have high drug:lipid ratios, stabilize both the lipid and nucleic acid components from degradation, protect the encapsulated nucleic acid from degradation and clearance in serum, are suitable for systemic delivery, and provide intracellular delivery of the encapsulated nucleic acid. In addition, these nucleic acid-lipid particles should be well-tolerated and provide an adequate therapeutic index, such that patient treatment at an effective dose of the nucleic acid is not associated with significant toxicity and/or risk to the patient. The present invention provides such compositions, methods of making them, and methods of using them to introduce nucleic acids into cells, including for the treatment of diseases.

### BRIEF SUMMARY OF THE INVENTION

The present invention provides methods of preventing, decreasing, or inhibiting the degradation of cationic lipids and/or active agents (e.g., therapeutic nucleic acids such as interfering RNA) present in lipid particles, compositions comprising lipid particles stabilized by these methods, methods of making these lipid particles, and methods of delivering and/or administering these lipid particles (e.g., for the treatment of a disease or disorder).

In one aspect, the invention provides a method for preventing, decreasing, or inhibiting the degradation of a cationic lipid present in a lipid particle, the method comprising:
including an antioxidant in the lipid particle, wherein the lipid particle comprises an active agent, the cationic lipid, a non-cationic lipid, and a conjugated lipid that inhibits aggregation of the particle.

In another aspect, the present invention provides a lipid particle composition, the composition comprising:
(a) a plurality of lipid particles comprising: an active agent; a cationic lipid; a non-cationic lipid; and a conjugated lipid that inhibits aggregation of the particle; and
(b) an antioxidant.

The antioxidant can be a hydrophilic antioxidant, a lipophilic antioxidant, a metal chelator, a primary antioxidant, a secondary antioxidant, salts thereof, and mixtures thereof. In certain embodiments, the antioxidant comprises a metal chelator such as EDTA or salts thereof, alone or in combination with one, two, three, four, five, six, seven, eight, or more additional antioxidants such as primary antioxidants, secondary antioxidants, or other metal chelators.

The cationic lipid component of the lipid particle can be a saturated cationic lipid, an unsaturated (e.g., monounsaturated and/or polyunsaturated) cationic lipid, or mixtures thereof. In some embodiments, the monounsaturated cationic lipid comprises a mixture of saturated and monounsaturated lipid moieties. In other embodiments, the polyunsaturated cationic lipid comprises a mixture of polyunsaturated lipid moieties with saturated and/or monounsaturated lipid moieties. In preferred embodiments, the cationic lipid component comprises one or more polyunsaturated cationic lipids, alone or in combination with one or more other cationic lipid species.

The active agent component of the lipid particle can be a nucleic acid, peptide, polypeptide, small molecule, or mixtures thereof. Non-limiting examples of nucleic acids include interfering RNA molecules (e.g., siRNA, Dicer-substrate dsRNA, shRNA, aiRNA, and/or miRNA), antisense oligonucleotides, plasmids, ribozymes, immunostimulatory oligonucleotides, and mixtures thereof. Examples of peptides or polypeptides include, without limitation, antibodies, cytokines, growth factors, apoptotic factors, differentiation-inducing factors, cell-surface receptors and their ligands, hormones, and mixtures thereof. Examples of small molecules include, but are not limited to, small organic molecules or compounds such as any conventional agent or drug known to those of skill in the art.

In some embodiments, the present invention provides a method for preventing, decreasing, or inhibiting the degradation of a cationic lipid present in a nucleic acid-lipid particle, the method comprising:
including an antioxidant in the nucleic acid-lipid particle, wherein the nucleic acid-lipid particle comprises a nucleic acid, the cationic lipid, a non-cationic lipid, and a conjugated lipid that inhibits aggregation of the particle.

In one particular embodiment, the invention provides a method for preventing, decreasing, or inhibiting the degradation of a polyunsaturated cationic lipid present in a nucleic acid-lipid particle, the method comprising:
including an antioxidant in the nucleic acid-lipid particle, wherein the antioxidant comprises ethylenediaminetetraacetic acid (EDTA) or a salt thereof, and
wherein the nucleic acid-lipid particle comprises a nucleic acid, the polyunsaturated cationic lipid, a non-cationic lipid, and a conjugated lipid that inhibits aggregation of the particle.

In another particular embodiment, the invention provides a method for preventing, decreasing, or inhibiting the degradation of a polyunsaturated cationic lipid present in a nucleic acid-lipid particle, the method comprising:
including an antioxidant in the nucleic acid-lipid particle, wherein the antioxidant comprises at least about 100 mM citrate or a salt thereof, and
wherein the nucleic acid-lipid particle comprises a nucleic acid, the polyunsaturated cationic lipid, a non-cationic lipid, and a conjugated lipid that inhibits aggregation of the particle.

In other embodiments, the present invention provides a nucleic acid-lipid particle composition, the composition comprising:
(a) a plurality of nucleic acid-lipid particles comprising: a nucleic acid; a cationic lipid; a non-cationic lipid; and a conjugated lipid that inhibits aggregation of the particle; and
(b) an antioxidant.

In one particular embodiment, the invention provides a nucleic acid-lipid particle composition, the composition comprising:
(a) a plurality of nucleic acid-lipid particles comprising: a nucleic acid; a polyunsaturated cationic lipid; a non-cationic lipid; and a conjugated lipid that inhibits aggregation of the particle; and
(b) an antioxidant, wherein the antioxidant comprises EDTA or a salt thereof.

In another particular embodiment, the invention provides a nucleic acid-lipid particle composition, the composition comprising:
(a) a plurality of nucleic acid-lipid particles comprising: a nucleic acid; a polyunsaturated cationic lipid; a non-cationic lipid; and a conjugated lipid that inhibits aggregation of the particle; and
(b) an antioxidant, wherein the antioxidant comprises at least about 100 mM citrate or a salt thereof.

In particular embodiments, the antioxidant can further comprise at least one, two, three, four, five, six, seven, eight, or more additional antioxidants including, but not limited to, primary antioxidants, secondary antioxidants, and other metal chelators. In one preferred embodiment, the antioxidant comprises a metal chelator such as EDTA or salts thereof in a mixture with one or more primary antioxidants and/or secondary antioxidants. For example, the antioxidant may comprise a mixture of EDTA or a salt thereof, a primary antioxidant such as α-tocopherol or a salt thereof, and a secondary antioxidant such as ascorbyl palmitate or a salt thereof.

In some instances, the nucleic acid-lipid particle comprises 1, 2, 3, 4, 5, 6, 7, 8, 9, 10, or more of unmodified and/or modified nucleic acid (e.g., interfering RNA) sequences. In certain instances, the nucleic acid-lipid particle comprises one or a cocktail (e.g., at least 2, 3, 4, 5, 6, 7, 8, 9, 10, or more) of 2′OMe-modified siRNA sequences.

In other instances, the nucleic acid (e.g., interfering RNA) component is fully encapsulated in the nucleic acid-lipid particle. With respect to formulations comprising an siRNA cocktail, the different types of siRNAs may be co-encapsulated in the same nucleic acid-lipid particle, or each type of siRNA species present in the cocktail may be encapsulated in its own nucleic acid-lipid particle.

The present invention also provides pharmaceutical compositions comprising a lipid particle, an antioxidant, and a pharmaceutically acceptable carrier.

The compositions and methods of the invention are useful for the delivery of therapeutic agents such as interfering RNA (e.g., siRNA) molecules that silence the expression of one or more genes. In some embodiments, one or a cocktail of siRNA molecules is formulated into the same or different nucleic acid-lipid particles, and the particles are administered to a mammal (e.g., a rodent such as a mouse or a primate such as a human, chimpanzee, or monkey) requiring such treatment. In certain instances, a therapeutically effective amount of the nucleic acid-lipid particles can be administered to the mammal, e.g., for treating a liver disorder such as dyslipidemia or for treating a cell proliferative disorder such as cancer. Administration of the nucleic acid-lipid particle formulation can be by any route known in the art, such as, e.g., oral, intranasal, intravenous, intraperitoneal, intramuscular, intra-articular, intralesional, intratracheal, subcutaneous, or intradermal.

Other objects, features, and advantages of the present invention will be apparent to one of skill in the art from the following detailed description and figures.

### BRIEF DESCRIPTION OF THE DRAWINGS

FIG. 1 illustrates an AX-HPLC chromatogram revealing degradation products in the SNALP phosphorothioate payload.

FIG. 2 illustrates an IPRP-HPLC chromatogram revealing siRNA payload conversion.

FIG. 3 illustrates a schematic of an exemplary SNALP formulation process.

FIG. 4 illustrates representative IPRP-HPLC traces from the data in Table 5. EDTA formulations inhibit the siRNA conversion apparent in the control (top trace).

FIG. 5 illustrates gene silencing efficacy of EDTA-4 SNALP. BALB/c mice were administered SNALP containing ApoB siRNA with phosphorothioate linkages as bolus tail vein injections at an siRNA dosage of 0.2 mg/kg. Liver ApoB mRNA was measured 48 h later using the QuantiGene assay (Panomics) and target gene data was normalized against GAPDH mRNA. Each bar represents an individual animal. Error bars represent the standard deviation of the mean of 2 replicate assay measurements. Gene silencing (KD) for each treatment is expressed as an animal group mean (n=4)±standard deviation.

FIG. 6 illustrates the body weight profile following EDTA-4 SNALP treatment. BALB/c mice (n=4) were administered SNALP as bolus tail vein injections at an siRNA dosage of 20 mg/kg. Body weight was measured just prior to dosing as well as 24 h and 48 h after treatment.

FIG. 7 illustrates the effect of EDTA on SNALP activity in a HepG2 cell model. HepG2 human hepatoma cells were exposed for 24 h to 2.5-80 nM SNALP containing ApoB siRNA with phosphorothioate linkages. 24 h after removal of transfection components, culture medium was collected and assayed for secreted human ApoB protein by ELISA. Cell treatments were performed in triplicate. Error bars indicate standard deviation of the mean.

FIG. 8 illustrates the in vivo gene silencing of EDTA-7 SNALP. BALB/c mice were administered SNALP containing ApoB siRNA with phosphorothioate linkages as bolus tail vein injections at an siRNA dosage of 0.2 mg/kg. Liver ApoB mRNA was measured 48 h later using the QuantiGene assay (Panomics) and target gene data was normalized against GAPDH mRNA. Each bar represents an individual animal. Gene silencing for each treatment is expressed as an animal group mean (n=4)±standard deviation.

FIG. 9 illustrates the body weight profile following EDTA-7 SNALP treatment. BALB/c mice (n=4) were administered SNALP as bolus tail vein injections at an siRNA dosage of 20 mg/kg. Body weight was measured just prior to dosing as well as 24 h and 48 h after treatment.

FIG. 10 illustrates rat liver enzymes following treatment with EDTA SNALP. Male Sprague-Dawley rats (n=2) were administered SNALP as bolus tail vein injections at an siRNA dosage of 5 mg/kg. Blood was collected via cardiac puncture for analysis at 24 h.

FIG. 11 illustrates an HPLC analysis of each of the lipid components present in SNALP over a period of 9 months at 5° C. when formulated with either 20 mM EDTA or 20 mM citrate.

FIG. 12 illustrates an HPLC analysis of each of the lipid components present in SNALP over a period of 5 months at room temperature when formulated with either 20 mM EDTA or 20 mM citrate.

FIG. 13 illustrates an HPLC analysis of the siRNA component present in SNALP when formulated with either 20 mM EDTA or 20 mM citrate.

FIG. 14 illustrates a particle size analysis of SNALP when formulated with either 20 mM EDTA or 20 mM citrate.

FIGS. 15-16 illustrate the results for Formulations 1-8 described in Example 3 with regard to particle size and percent PO content over a 1 month period. For ascorbyl palmitate (AP) and α-tocopherol: “−” means 0.1 mol %; “+” means 1.0 mol %. For EDTA: “−” means 20 mM EDTA; “+” means 80 mM EDTA. The table at the top of each figure shows statistical significance.

### DETAILED DESCRIPTION OF THE INVENTION

### Introduction

Krotz et al. (J. Pharm. Sci., 94:341-352 (2005)) describes the desulfurization of phosphorothioate (PS) linkages in antisense oligonucleotides (ASO) formulated as oil-in-water emulsions. Krotz et al. indicates that the cause of desulfurization was related to the presence of the PEG-derived nonionic surfactants MYRJ 52 or BRIG 58. Krotz et al. discloses that only L-cysteine or DL-α-lipoic acid resulted in minimal desulfurization of PS-modified ASO in oil-in-water emulsions. In fact, Krotz et al. teaches that EDTA actually significantly accelerated the desulfurization of PS-modified ASO in the presence of MYRJ 52. Similarly, US Patent Publication No. 2005/0208528 describes the inhibition of desulfurization of PS-modified ASO in a bi-phasic cream formulation with L-cysteine, glutathione, α-lipoic acid, or 2-mercaptobenzimidazole sulfonic acid, sodium salt.

In stark contrast, the present invention is based in part on the surprising discovery that the presence of the antioxidant EDTA (or a salt thereof), a high concentration of the antioxidant citrate (or a salt thereof), or EDTA (or a salt thereof) in combination with one or more (e.g., a mixture of) primary and/or secondary antioxidants such as α-tocopherol (or a salt thereof) and/or ascorbyl palmitate (or a salt thereof) protects the nucleic acid payload and the polyunsaturated cationic lipid component of a nucleic acid-lipid particle (e.g., SNALP) from degradation. The bis-allylic methylene (CH₂) groups of polyunsaturated lipids have weak carbon-hydrogen bonds and are prone to hydrogen abstraction by heat/light energy or radical species resulting in reactive lipid radicals. Lipid radicals may combine with molecular oxygen to form lipid hydroperoxide, a strong oxidant, or transfer the radical to another molecule (i.e., radical propagation). See, Wagner et al., Biochemistry, 33:4449-53 (1994).

The present inventors have found that the presence of polyunsaturated cationic lipids in the nucleic acid-lipid particle (e.g., SNALP) may cause degradation of the nucleic acid payload regardless of whether it contains PS linkages in the sequence. In addition to the desulfurization/conversion of PS, nucleic acid and polyunsaturated cationic lipid degradation is observed irrespective of whether the nucleic acid sequence contains any PS modifications.

However, the present inventors have unexpectedly discovered that the antioxidant EDTA (or a salt thereof), a high concentration of the antioxidant citrate (or a salt thereof), or EDTA (or a salt thereof) in combination with primary and/or secondary antioxidants such as α-tocopherol (or a salt thereof) and/or ascorbyl palmitate (or a salt thereof) advantageously protects the polyunsaturated cationic lipid component of the nucleic acid-lipid particle from degradation. In particular, Examples 1-3 below demonstrate that incorporation of an antioxidant or a mixture thereof into the SNALP formulation provides at least one of the following advantages: (1) the antioxidant or mixture thereof decreases or prevents the oxidation of the polyunsaturated cationic lipid; (2) the antioxidant or mixture thereof reduces or prevents the degradation of the nucleic acid payload; (3) the antioxidant or mixture thereof stabilizes both the lipid and nucleic acid components over time at all temperatures tested; and/or (4) the antioxidant or mixture thereof reduces or prevents the desulfurization of a PS-modified nucleic acid payload. Furthermore, these Examples show that SNALP formulations containing antioxidants are well-tolerated and display potencies similar to that observed for control SNALP formulations.

As such, the nucleic acid-lipid particle formulations of the present invention, which comprise lipid and nucleic acid components that are stable and are protected from oxidative degradation, have the ability to safely and effectively deliver a nucleic acid payload such as an interfering RNA (e.g., siRNA) to a target cell, tissue, tumor, and/or organ without having any negative impact on silencing activity.

### DEFINITIONS

As used herein, the following terms have the meanings ascribed to them unless specified otherwise.

The term “antioxidant” includes any molecule capable of slowing, reducing, inhibiting, or preventing the oxidation of other molecules. Oxidation is a chemical reaction that transfers electrons from a substance to an oxidizing agent. Oxidation reactions can produce free radicals, which are highly reactive chemicals that attack molecules by capturing electrons and thus modifying chemical structures. Antioxidants remove free radical intermediates and inhibit other oxidation reactions by being oxidized themselves. Examples of antioxidants include, but are not limited to, hydrophilic antioxidants, lipophilic antioxidants, and mixtures thereof. Non-limiting examples of hydrophilic antioxidants include chelating agents (e.g., metal chelators) such as ethylenediaminetetraacetic acid (EDTA), citrate, ethylene glycol tetraacetic acid (EGTA), 1,2-bis(o-aminophenoxy)ethane-N,N,N′,N′-tetraacetic acid (BAPTA), diethylene triamine pentaacetic acid (DTPA), 2,3-dimercapto-1-propanesulfonic acid (DMPS), dimercaptosuccinic acid (DMSA), α-lipoic acid, salicylaldehyde isonicotinoyl hydrazone (SIH), hexyl thioethylamine hydrochloride (HTA), desferrioxamine, salts thereof, and mixtures thereof. Additional hydrophilic antioxidants include ascorbic acid, cysteine, glutathione, dihydrolipoic acid, 2-mercaptoethane sulfonic acid, 2-mercaptobenzimidazole sulfonic acid, 6-hydroxy-2,5,7,8-tetramethylchroman-2-carboxylic acid, sodium metabisulfite, salts thereof, and mixtures thereof. Non-limiting examples of lipophilic antioxidants include vitamin E isomers such as α-, β-, γ-, and δ-tocopherols and α-, β-, γ-, and δ-tocotrienols; polyphenols such as 2-tert-butyl-4-methyl phenol, 2-tert-butyl-5-methyl phenol, and 2-tert-butyl-6-methyl phenol; butylated hydroxyanisole (BHA) (e.g., 2-tert-butyl-4-hydroxyanisole and 3-tert-butyl-4-hydroxyanisole); butylhydroxytoluene (BHT); tert-butylhydroquinone (TBHQ); ascorbyl palmitate; n-propyl gallate; salts thereof; and mixtures thereof. One of skill in the art will appreciate that antioxidants can be classified as primary antioxidants, secondary antioxidants, or metal chelators based upon the mechanisms in which they act. Primary antioxidants quench free radicals which are often the source of oxidative pathways, whereas secondary antioxidants function by decomposing the peroxides that are reactive intermediates of the pathways. Metal chelators function by sequestering the trace metals that promote free radical development. Table 1 provides exemplary antioxidants which belong to one or more of these classes:

TABLE 1

| | |
|---|---|
| Primary antioxidants | Vitamin E isomers (e.g., α-, β-, γ-, δ- |
| (radical scavengers) | tocopherols; α-, β-, γ-, δ-tocotrienols), |
| | BHA, BHT, TBHQ |
| Secondary antioxidants | Ascorbic acid, ascorbyl palmitate, |
| (oxygen scavengers, reductants) | cysteine, glutathione, α-lipoic acid |
| Metal chelators | EDTA, citrate, α-lipoic acid, DTPA, |
| | SIH, HTA, desferrioxamine |

In particular embodiments, the antioxidant (e.g., one or a mixture of primary antioxidants, secondary antioxidants, and metal chelators) is capable of preventing, inhibiting, or retarding the oxidative degradation of the (e.g., polyunsaturated) cationic lipid component of a nucleic acid-lipid particle (e.g., SNALP).

The term “interfering RNA” or “RNAi” or “interfering RNA sequence” as used herein includes single-stranded RNA (e.g., mature miRNA, ssRNAi oligonucleotides, ssDNAi oligonucleotides), double-stranded RNA (i.e., duplex RNA such as siRNA, Dicer-substrate dsRNA, shRNA, aiRNA, or pre-miRNA), a DNA-RNA hybrid (see, e.g., PCT Publication No. WO 2004/078941), or a DNA-DNA hybrid (see, e.g., PCT Publication No. WO 2004/104199) that is capable of reducing or inhibiting the expression of a target gene or sequence (e.g., by mediating the degradation or inhibiting the translation of mRNAs which are complementary to the interfering RNA sequence) when the interfering RNA is in the same cell as the target gene or sequence. Interfering RNA thus refers to the single-stranded RNA that is complementary to a target mRNA sequence or to the double-stranded RNA formed by two complementary strands or by a single, self-complementary strand. Interfering RNA may have substantial or complete identity to the target gene or sequence, or may comprise a region of mismatch (i.e., a mismatch motif). The sequence of the interfering RNA can correspond to the full-length target gene, or a subsequence thereof. Preferably, the interfering RNA molecules are chemically synthesized. The disclosures of each of the above patent documents are herein incorporated by reference in their entirety for all purposes.

Interfering RNA includes “small-interfering RNA” or “siRNA,” e.g., interfering RNA of about 15-60, 15-50, or 15-40 (duplex) nucleotides in length, more typically about 15-30, 15-25, or 19-25 (duplex) nucleotides in length, and is preferably about 20-24, 21-22, or 21-23 (duplex) nucleotides in length (e.g., each complementary sequence of the double-stranded siRNA is 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 15-30, 15-25, or 19-25 base pairs in length, preferably about 18-22, 19-20, or 19-21 base pairs in length). siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand; a double-stranded polynucleotide molecule assembled from a single stranded molecule, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule. As used herein, the term “siRNA” includes RNA-RNA duplexes as well as DNA-RNA hybrids (see, e.g., PCT Publication No. WO 2004/078941).

Preferably, siRNA are chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., Proc. Natl. Acad. Sci. USA, 99:9942-9947 (2002); Calegari et al., Proc. Natl. Acad. Sci. USA, 99:14236 (2002); Byrom et al., Ambion TechNotes, 10(1):4-6 (2003); Kawasaki et al., Nucleic Acids Res., 31:981-987 (2003); Knight et al., Science, 293:2269-2271 (2001); and Robertson et al., J. Biol. Chem., 243:82 (1968)). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

As used herein, the term “mismatch motif” or “mismatch region” refers to a portion of an interfering RNA (e.g., siRNA) sequence that does not have 100% complementarity to its target sequence. An interfering RNA may have at least one, two, three, four, five, six, or more mismatch regions. The mismatch regions may be contiguous or may be separated by 1, 2, 3, 4, 5, 6, 7, 8, 9, 10, 11, 12, or more nucleotides. The mismatch motifs or regions may comprise a single nucleotide or may comprise two, three, four, five, or more nucleotides.

The phrase “inhibiting expression of a target gene” refers to the ability of a nucleic acid such as an interfering RNA (e.g., siRNA) to silence, reduce, or inhibit the expression of a target gene. To examine the extent of gene silencing, a test sample (e.g., a sample of cells in culture expressing the target gene) or a test mammal (e.g., a mammal such as a human or an animal model such as a rodent (e.g., mouse) or a non-human primate (e.g., monkey) model) is contacted with a nucleic acid such as an interfering RNA (e.g., siRNA) that silences, reduces, or inhibits expression of the target gene. Expression of the target gene in the test sample or test animal is compared to expression of the target gene in a control sample (e.g., a sample of cells in culture expressing the target gene) or a control mammal (e.g., a mammal such as a human or an animal model such as a rodent (e.g., mouse) or non-human primate (e.g., monkey) model) that is not contacted with or administered the nucleic acid (e.g., interfering RNA). The expression of the target gene in a control sample or a control mammal may be assigned a value of 100%. In particular embodiments, silencing, inhibition, or reduction of expression of a target gene is achieved when the level of target gene expression in the test sample or the test mammal relative to the level of target gene expression in the control sample or the control mammal is about 95%, 90%, 85%, 80%, 75%, 70%, 65%, 60%, 55%, 50%, 45%, 40%, 35%, 30%, 25%, 20%, 15%, 10%, 5%, or 0%. In other words, the nucleic acids (e.g., interfering RNAs) are capable of silencing, reducing, or inhibiting the expression of a target gene by at least about 5%, 10%, 15%, 20%, 25%, 30%, 35%, 40%, 45%, 50%, 55%, 60%, 65%, 70%, 75%, 80%, 85%, 90%, 95%, or 100% in a test sample or a test mammal relative to the level of target gene expression in a control sample or a control mammal not contacted with or administered the nucleic acid (e.g., interfering RNA). Suitable assays for determining the level of target gene expression include, without limitation, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

An “effective amount” or “therapeutically effective amount” of a therapeutic nucleic acid such as an interfering RNA (e.g., siRNA) is an amount sufficient to produce the desired effect, e.g., an inhibition of expression of a target sequence in comparison to the normal expression level detected in the absence of the nucleic acid (e.g., interfering RNA). Inhibition of expression of a target gene or target sequence is achieved when the value obtained with a nucleic acid such as an interfering RNA (e.g., siRNA) relative to the control is about 95%, 90%, 85%, 80%, 75%, 70%, 65%, 60%, 55%, 50%, 45%, 40%, 35%, 30%, 25%, 20%, 15%, 10%, 5%, or 0%. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, as well as phenotypic assays known to those of skill in the art.

By “decrease,” “decreasing,” “reduce,” or “reducing” of an immune response by a nucleic acid such as an interfering RNA (e.g., siRNA) is intended to mean a detectable decrease of an immune response to a given nucleic acid (e.g., a modified interfering RNA). In some instances, the amount of decrease of an immune response by a nucleic acid such as a modified interfering RNA may be determined relative to the level of an immune response in the presence of an unmodified interfering RNA. As a non-limiting example, a detectable decrease can be about 5%, 10%, 15%, 20%, 25%, 30%, 35%, 40%, 45%, 50%, 55%, 60%, 65%, 70%, 75%, 80%, 85%, 90%, 95%, 100%, or more lower than the immune response detected in the presence of the unmodified interfering RNA. A decrease in the immune response to a nucleic acid (e.g., interfering RNA) is typically measured by a decrease in cytokine production (e.g., IFNγ, IFNα, TNFα, IL-6, IL-8, or IL-12) by a responder cell in vitro or a decrease in cytokine production in the sera of a mammalian subject after administration of the nucleic acid (e.g., interfering RNA).

As used herein, the term “responder cell” refers to a cell, preferably a mammalian cell, that produces a detectable immune response when contacted with an immunostimulatory nucleic acid such as an unmodified interfering RNA (e.g., unmodified siRNA). Exemplary responder cells include, without limitation, dendritic cells, macrophages, peripheral blood mononuclear cells (PBMCs), splenocytes, and the like. Detectable immune responses include, e.g., production of cytokines or growth factors such as TNF-α, IFN-α, IFN-β, IFN-γ, IL-1, IL-2, IL-3, IL-4, IL-5, IL-6, IL-8, IL-10, IL-12, IL-13, TGF, and combinations thereof. Detectable immune responses also include, e.g., induction of interferon-induced protein with tetratricopeptide repeats 1 (IFIT1) mRNA.

The term “nucleic acid” as used herein refers to a polymer containing at least two deoxyribonucleotides or ribonucleotides in either single- or double-stranded form and includes DNA, RNA, and hybrids thereof DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. RNA may be in the form of small interfering RNA (siRNA), Dicer-substrate dsRNA, small hairpin RNA (shRNA), asymmetrical interfering RNA (aiRNA), microRNA (miRNA), mRNA, tRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The term “lipid particle” includes a lipid formulation that can be used to deliver a therapeutic nucleic acid (e.g., interfering RNA) to a target site of interest (e.g., cell, tissue, organ, tumor, and the like). In preferred embodiments, the lipid particle of the invention is a nucleic acid-lipid particle, which is typically formed from a cationic lipid, a non-cationic lipid, and optionally a conjugated lipid that prevents aggregation of the particle. In other preferred embodiments, the therapeutic nucleic acid (e.g., interfering RNA) may be encapsulated in the lipid portion of the particle, thereby protecting it from enzymatic degradation.

As used herein, the term “SNALP” refers to a stable nucleic acid-lipid particle. A SNALP represents a particle made from lipids (e.g., a cationic lipid, a non-cationic lipid, and optionally a conjugated lipid that prevents aggregation of the particle), wherein the nucleic acid (e.g., an interfering RNA) is fully encapsulated within the lipid. In certain instances, SNALP are extremely useful for systemic applications, as they can exhibit extended circulation lifetimes following intravenous (i.v.) injection, they can accumulate at distal sites (e.g., sites physically separated from the administration site), and they can mediate silencing of target gene expression at these distal sites. The nucleic acid may be complexed with a condensing agent and encapsulated within a SNALP as set forth in PCT Publication No. WO 00/03683, the disclosure of which is herein incorporated by reference in its entirety for all purposes.

The lipid particles of the invention (e.g., SNALP) typically have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In addition, nucleic acids, when present in the lipid particles of the present invention, are resistant in aqueous solution to degradation with a nuclease. Nucleic acid-lipid particles and their method of preparation are disclosed in, e.g., U.S. Patent Publication Nos. 20040142025 and 20070042031, the disclosures of which are herein incorporated by reference in their entirety for all purposes.

As used herein, “lipid encapsulated” can refer to a lipid particle that provides a therapeutic nucleic acid, such as an interfering RNA (e.g., siRNA), with full encapsulation, partial encapsulation, or both. In a preferred embodiment, the nucleic acid (e.g., interfering RNA) is fully encapsulated in the lipid particle (e.g., to form a SNALP or other nucleic acid-lipid particle).

The term “lipid conjugate” refers to a conjugated lipid that inhibits aggregation of lipid particles. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., U.S. Provisional Application No. 61/294,828, filed Jan. 13, 2010, and U.S. Provisional Application No. 61/295,140, filed Jan. 14, 2010), polyamide oligomers (e.g., ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes.

The term “amphipathic lipid” refers, in part, to any suitable material wherein the hydrophobic portion of the lipid material orients into a hydrophobic phase, while the hydrophilic portion orients toward the aqueous phase. Hydrophilic characteristics derive from the presence of polar or charged groups such as carbohydrates, phosphate, carboxylic, sulfato, amino, sulfhydryl, nitro, hydroxyl, and other like groups. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). Examples of amphipathic compounds include, but are not limited to, phospholipids, aminolipids, and sphingolipids.

Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and β-acyloxyacids, are also within the group designated as amphipathic lipids. Additionally, the amphipathic lipids described above can be mixed with other lipids including triglycerides and sterols.

The term “neutral lipid” refers to any of a number of lipid species that exist either in an uncharged or neutral zwitterionic form at a selected pH. At physiological pH, such lipids include, for example, diacylphosphatidylcholines, diacylphosphatidylethanolamine, ceramide, sphingomyelin, cephalin, cholesterol, cerebrosides, and diacylglycerols.

The term “non-cationic lipid” refers to any amphipathic lipid as well as any other neutral lipid or anionic lipid.

The term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “hydrophobic lipid” refers to compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N—N-dialkylamino, 1,2-diacyloxy-3-aminopropane, and 1,2-dialkyl-3-aminopropane.

The term “fusogenic” refers to the ability of a lipid particle, such as a SNALP, to fuse with the membranes of a cell. The membranes can be either the plasma membrane or membranes surrounding organelles, e.g., endosome, nucleus, etc.

As used herein, the term “aqueous solution” refers to a composition comprising in whole, or in part, water.

As used herein, the term “organic lipid solution” refers to a composition comprising in whole, or in part, an organic solvent having a lipid.

“Distal site,” as used herein, refers to a physically separated site, which is not limited to an adjacent capillary bed, but includes sites broadly distributed throughout an organism.

“Serum-stable” in relation to nucleic acid-lipid particles such as SNALP means that the particle is not significantly degraded after exposure to a serum or nuclease assay that would significantly degrade free DNA or RNA. Suitable assays include, for example, a standard serum assay, a DNAse assay, or an RNAse assay.

“Systemic delivery,” as used herein, refers to delivery of lipid particles that leads to a broad biodistribution of an active agent such as an interfering RNA (e.g., siRNA) within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of lipid particles can be by any means known in the art including, for example, intravenous, subcutaneous, and intraperitoneal. In a preferred embodiment, systemic delivery of lipid particles is by intravenous delivery.

“Local delivery,” as used herein, refers to delivery of an active agent such as an interfering RNA (e.g., siRNA) directly to a target site within an organism. For example, an agent can be locally delivered by direct injection into a disease site such as a tumor, other target site, or a target organ such as the liver, heart, pancreas, kidney, and the like.

The term “mammal” refers to any mammalian species such as a human, mouse, rat, dog, cat, hamster, guinea pig, rabbit, livestock, and the like.

The terms “cationic lipid” and “amino lipid” are used interchangeably herein to include those lipids and salts thereof having one, two, three, or more fatty acid or fatty alkyl chains and a pH-titratable amino head group (e.g., an alkylamino or dialkylamino head group). The cationic lipid is typically protonated (i.e., positively charged) at a pH below the pKₐ of the cationic lipid and is substantially neutral at a pH above the pKₐ. The cationic lipids of the invention may also be termed titratable cationic lipids.

A “polyunsaturated cationic lipid” includes those lipids and salts thereof having one, two, three, or more fatty acid or fatty alkyl chains and a pH-titratable amino head group (e.g., an alkylamino or dialkylamino head group), wherein at least one, two, three, or more of the fatty acid or fatty alkyl chains independently comprises at least two, three, four, five, six, or more sites of unsaturation (i.e., double bonds). In some embodiments, the polyunsaturated cationic lipids comprise: a protonatable tertiary amine (e.g., pH-titratable) head group; C₁₈ alkyl chains, wherein each alkyl chain independently has 2 or 3 double bonds; and linkages between the head group and alkyl chains as described herein. Such polyunsaturated cationic lipids include, but are not limited to, DLinDMA, DLenDMA, γ-DLenDMA, DLin-K-C2-DMA, DLin-K-DMA, DLin-M-C3-DMA, MC3 Ether, MC4 Ether, DLen-C2K-DMA, γ-DLen-C2K-DMA, and mixtures thereof.

A “monounsaturated cationic lipid” includes those lipids and salts thereof having one, two, three, or more fatty acid or fatty alkyl chains and a pH-titratable amino head group (e.g., an alkylamino or dialkylamino head group), wherein none of the fatty acid or fatty alkyl chains comprises more than one site of unsaturation (i.e., a double bond).

The term “salts” includes any anionic and cationic complex. Non-limiting examples of anions include inorganic and organic anions, e.g., hydride, fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof. Examples of cations include, but are not limited to, aluminum, calcium, copper(II), iron(II), iron(III), magnesium, mercury(II), potassium, silver, sodium, ammonium, hydronium, mercury(I), and mixtures thereof (e.g., calcium disodium). In certain instances, the term “salt” includes a complex formed between a cationic lipid and one or more anions. In some particular embodiments, the salts of the cationic lipids disclosed herein are crystalline salts. In other instances, the term “salt” includes a complex formed between an antioxidant and one or more cations or anions. In particular embodiments, the salts of EDTA disclosed herein are calcium disodium salts.

The term “a plurality of nucleic acid-lipid particles” refers to at least 2 particles, more preferably more than 10, 10², 10³, 10⁴, 10⁵, 10⁶ or more particles (or any fraction thereof or range therein). In certain embodiments, the plurality of nucleic acid-lipid particles includes 50-100, 50-200, 50-300, 50-400, 50-500, 50-600, 50-700, 50-800, 50-900, 50-1000, 50-1100, 50-1200, 50-1300, 50-1400, 50-1500, 50-1600, 50-1700, 50-1800, 50-1900, 50-2000, 50-2500, 50-3000, 50-3500, 50-4000, 50-4500, 50-5000, 50-5500, 50-6000, 50-6500, 50-7000, 50-7500, 50-8000, 50-8500, 50-9000, 50-9500, 50-10,000 or more particles. It will be apparent to those of skill in the art that the plurality of nucleic acid-lipid particles can include any fraction of the foregoing ranges or any range therein.

### DESCRIPTION OF THE EMBODIMENTS

The present invention provides methods of preventing, decreasing, or inhibiting the degradation of cationic lipids and/or active agents (e.g., therapeutic nucleic acids) present in lipid particles, compositions comprising lipid particles stabilized by these methods, methods of making these lipid particles, and methods of delivering and/or administering these lipid particles (e.g., for the treatment of a disease or disorder).

In one aspect, the invention provides a method for preventing, decreasing, or inhibiting the degradation of a cationic lipid present in a lipid particle, the method comprising:
including an antioxidant in the lipid particle, wherein the lipid particle comprises an active agent, the cationic lipid, a non-cationic lipid, and a conjugated lipid that inhibits aggregation of the particle.

In particular embodiments, the step of including an antioxidant in the lipid particle comprises contacting the active agent (e.g., nucleic acid) with at least one antioxidant and/or contacting a lipid stock (e.g., an organic lipid solution containing the lipid components of the particle solubilized therein) comprising the cationic lipid (e.g., polyunsaturated cationic lipid) with at least one antioxidant prior to formation of the lipid particle.

In a related aspect, the present invention provides a lipid particle composition, the composition comprising:
(a) a plurality of lipid particles comprising: an active agent; a cationic lipid; a non-cationic lipid; and a conjugated lipid that inhibits aggregation of the particle; and
(b) an antioxidant.

In certain preferred embodiments, the antioxidant is present in an amount sufficient to prevent, inhibit, or reduce the degradation of the cationic lipid present in the lipid particle.

Exemplary concentrations or ranges of concentrations for an individual antioxidant species or for a combination of antioxidants include, but are not limited to, at least about or about 0.1 mM, 0.5 mM, 1 mM, 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 200 mM, 250 mM, 300 mM, 400 mM, 500 mM, 600 mM, 700 mM, 800 mM, 900 mM, 1 M, 2 M, and 5M, or from about 0.1 mM to about 1 M, from about 0.1 mM to about 500 mM, from about 0.1 mM to about 250 mM, from about 0.1 mM to about 100 mM, from about 50 mM to about 500 mM, from about 50 mM to about 250 mM, from about 50 mM to about 150 mM, from about 100 mM to about 500 mM, from about 100 mM to about 250 mM, from about 5 mM to about 500 mM, from about 5 mM to about 250 mM, from about 5 mM to about 100 mM, from about 20 mM to about 100 mM, from about 5 mM to about 50 mM, from about 5 mM to about 25 mM, from about 10 mM to about 50 mM, from about 10 mM to about 30 mM, and from about 15 mM to about 25 mM.

Additional exemplary concentrations or ranges of concentrations for an individual antioxidant species or for a combination of antioxidants include, but are not limited to, at least about or about 0.01 mol %, 0.02 mol %, 0.05 mol %, 0.08 mol %, 0.1 mol %, 0.2 mol %, 0.3 mol %, 0.4 mol %, 0.5 mol %, 0.6 mol %, 0.7 mol %, 0.8 mol %, 0.9 mol %, 1.0 mol %, 1.2 mol %, 1.5 mol %, 1.8 mol %, 2.0 mol %, 2.5 mol %, 3.0 mol %, 3.5 mol %, 4.0 mol %, 4.5 mol %, 5.0 mol %, 5.5 mol %, 6.0 mol %, 6.5 mol %, 7.0 mol %, 7.5 mol %, 8.0 mol %, 8.5 mol %, 9.0 mol %, 9.5 mol %, 10.0 mol %, 12.5 mol %, 15.0 mol %, 17.5 mol %, 20.0 mol %, and 25.0 mol %, or from about 0.01 mol % to about 25.0 mol %, from about 0.01 mol % to about 10.0 mol %, from about 0.01 mol % to about 5.0 mol %, from about 0.01 mol % to about 1.0 mol %, from about 0.01 mol % to about 0.5 mol %, from about 0.02 mol % to about 10.0 mol %, from about 0.02 mol % to about 5.0 mol %, from about 0.02 mol % to about 1.0 mol %, from about 0.02 mol % to about 0.5 mol %, from about 0.05 mol % to about 10.0 mol %, from about 0.05 mol % to about 5.0 mol %, from about 0.05 mol % to about 1.0 mol %, from about 0.05 mol % to about 0.5 mol %, from about 0.05 mol % to about 0.2 mol %, from about 0.1 mol % to about 10.0 mol %, from about 0.1 mol % to about 5.0 mol %, from about 0.1 mol % to about 1.0 mol %, from about 0.1 mol % to about 0.5 mol %, from about 0.1 mol % to about 0.2 mol %, from about 0.2 mol % to about 10.0 mol %, from about 0.2 mol % to about 5.0 mol %, from about 0.2 mol % to about 1.0 mol %, from about 0.2 mol % to about 0.5 mol %, from about 0.5 mol % to about 10.0 mol %, from about 0.5 mol % to about 5.0 mol %, from about 0.5 mol % to about 2.0 mol %, and from about 0.5 mol % to about 1.0 mol %.

In some embodiments, the present invention provides a method for preventing, decreasing, or inhibiting the degradation of a cationic lipid present in a nucleic acid-lipid particle, the method comprising:
including an antioxidant in the nucleic acid-lipid particle, wherein the nucleic acid-lipid particle comprises a nucleic acid, the cationic lipid, a non-cationic lipid, and a conjugated lipid that inhibits aggregation of the particle.

In related embodiments, the present invention provides a nucleic acid-lipid particle composition, the composition comprising:
(a) a plurality of nucleic acid-lipid particles comprising: a nucleic acid; a cationic lipid; a non-cationic lipid; and a conjugated lipid that inhibits aggregation of the particle; and
(b) an antioxidant.

In one particular embodiment, the invention provides a method for preventing, decreasing, or inhibiting the degradation of a polyunsaturated cationic lipid present in a nucleic acid-lipid particle, the method comprising:
including an antioxidant in the nucleic acid-lipid particle, wherein the antioxidant comprises ethylenediaminetetraacetic acid (EDTA) or a salt thereof, and
wherein the nucleic acid-lipid particle comprises a nucleic acid, the polyunsaturated cationic lipid, a non-cationic lipid, and a conjugated lipid that inhibits aggregation of the particle.

In one related embodiment, the invention provides a nucleic acid-lipid particle composition, the composition comprising:
(a) a plurality of nucleic acid-lipid particles comprising: a nucleic acid; a polyunsaturated cationic lipid; a non-cationic lipid; and a conjugated lipid that inhibits aggregation of the particle; and
(b) an antioxidant, wherein the antioxidant comprises EDTA or a salt thereof.

The EDTA or salt thereof (e.g., sodium EDTA, calcium EDTA, and/or calcium disodium EDTA) can be present in any of the exemplary concentrations or concentration ranges described above, provided that the amount of the EDTA or salt thereof is sufficient to prevent, inhibit, or reduce the degradation of the cationic lipid present in the lipid particle. Preferably, the method or composition of the present invention comprises including at least about 20 mM EDTA or a salt thereof in the particle.

In particular embodiments, the method or composition further comprises including at least one, two, three, four, five, six, seven, eight, or more additional antioxidants in the particle. Examples of additional antioxidants include, without limitation, one or more of the hydrophilic and/or lipophilic antioxidants described herein or known in the art. For example, the method or composition of the invention can comprise including EDTA or a salt thereof (e.g., about 20 mM EDTA or a salt thereof) in combination with one, two, three, four, five, or more of the primary antioxidants, secondary antioxidants, and/or other metal chelators (or salts thereof) set forth in Table 1.

Non-limiting examples of primary antioxidants include a vitamin E isomer (e.g., α-tocopherol or a salt thereof), butylated hydroxyanisole (BHA), butylhydroxytoluene (BHT), tert-butylhydroquinone (TBHQ), salts thereof, and combinations thereof. Examples of secondary antioxidants include, but are not limited to, ascorbic acid, ascorbyl palmitate, cysteine, glutathione, α-lipoic acid, salts thereof, and combinations thereof.

The primary and/or secondary antioxidant can be present in any of the exemplary concentrations or concentration ranges described above, provided that the amount of the primary and/or secondary antioxidant in combination with the EDTA or salt thereof is sufficient to prevent, inhibit, or reduce the degradation of the cationic lipid present in the lipid particle. In preferred embodiments, the method or composition of the present invention comprises including EDTA or a salt thereof and from about 0.01 mol % to about 10.0 mol % of the primary and/or said secondary antioxidant in the particle. In certain instances, the primary and/or said secondary antioxidant is each independently included at a concentration of from about 0.01 mol % to about 10.0 mol %, preferably from about 0.05 mol % to about 5.0 mol %.

In one particular embodiment, the additional antioxidant comprises a mixture of a primary antioxidant or a salt thereof and a secondary antioxidant or a salt thereof. In some preferred embodiments, the mixture comprises α-tocopherol or a salt thereof and ascorbyl palmitate or a salt thereof. As a non-limiting example, in certain preferred embodiments, EDTA or salt thereof is included at a concentration of from about 20 mM to about 100 mM (e.g., preferably about 20 mM of an EDTA salt), α-tocopherol or a salt thereof is included at a concentration of from about 0.02 mol % to about 0.5 mol % (e.g., preferably from about 0.05 mol % to about 0.25 mol % or about 0.1 mol %), and ascorbyl palmitate or a salt thereof is included at a concentration of from about 0.02 mol % to about 5.0 mol % (e.g., preferably from about 0.05 mol % to about 2.5 mol %, about 0.1 mol %, or about 1.0 mol %).

In another particular embodiment, the invention provides a method for preventing, decreasing, or inhibiting the degradation of a polyunsaturated cationic lipid present in a nucleic acid-lipid particle, the method comprising:
including an antioxidant in the nucleic acid-lipid particle, wherein the antioxidant comprises at least about 100 mM (e.g., about 100 mM or more) citrate or a salt thereof, and
wherein the nucleic acid-lipid particle comprises a nucleic acid, the polyunsaturated cationic lipid, a non-cationic lipid, and a conjugated lipid that inhibits aggregation of the particle.

In another related embodiment, the invention provides a nucleic acid-lipid particle composition, the composition comprising:
(a) a plurality of nucleic acid-lipid particles comprising: a nucleic acid; a polyunsaturated cationic lipid; a non-cationic lipid; and a conjugated lipid that inhibits aggregation of the particle; and
(b) an antioxidant, wherein the antioxidant comprises at least about 100 mM citrate or a salt thereof.

In alternative embodiments, the citrate or salt thereof can be present in any of the exemplary concentrations or concentration ranges described above, provided that the amount of the citrate or salt thereof is sufficient to prevent, inhibit, or reduce the degradation of the cationic lipid present in the particle.

In particular embodiments, the method or composition further comprises including at least one, two, three, four, five, six, seven, eight, or more additional antioxidants in the particle. Examples of additional antioxidants include, without limitation, one or more of the hydrophilic and/or lipophilic antioxidants described herein or known in the art. For example, the method or composition of the invention can comprise including citrate or a salt thereof (e.g., at least about 100 mM or a salt thereof) in combination with one, two, three, four, five, or more of the primary antioxidants, secondary antioxidants, and/or other metal chelators (or salts thereof) set forth in Table 1.

The cationic lipid component of the nucleic acid-lipid particle can be a saturated cationic lipid, an unsaturated (e.g., monounsaturated and/or polyunsaturated) cationic lipid, or mixtures thereof. In some embodiments, the monounsaturated cationic lipid comprises a mixture of saturated and monounsaturated lipid moieties. In other embodiments, the polyunsaturated cationic lipid comprises a mixture of polyunsaturated lipid moieties with saturated and/or monounsaturated lipid moieties. In preferred embodiments, the cationic lipid comprises one or more polyunsaturated cationic lipids, alone or in combination with one or more other cationic lipid species.

In some embodiments, the polyunsaturated cationic lipid comprises at least one lipid moiety having at least two or at least three sites of unsaturation. In certain instances, at least one of the lipid moieties comprises a dodecadienyl moiety, a tetradecadienyl moiety, a hexadecadienyl moiety, an octadecadienyl moiety, an icosadienyl moiety, a dodecatrienyl moiety, a tetradectrienyl moiety, a hexadecatrienyl moiety, an octadecatrienyl moiety, an icosatrienyl moiety, an arachidonyl moiety, a docosahexaenoyl moiety, or combinations thereof. In preferred embodiments, at least one of the polyunsaturated lipid moieties comprises an octadecadienyl moiety (e.g., a linoleyl moiety), an octadecatrienyl moiety (e.g., a linolenyl moiety or a γ-linolenyl moiety), or combinations thereof. In another particular embodiment, the polyunsaturated cationic lipid comprises a combination of at least one polyunsaturated lipid moiety with at least one lipid moiety independently selected from the group consisting of an optionally substituted C₁-C₂₄ alkyl moiety, an optionally substituted C₂-C₂₄ alkenyl moiety, an optionally substituted C₂-C₂₄ alkynyl moiety, an optionally substituted C₁-C₂₄ acyl moiety, and mixtures thereof.

In other embodiments, the polyunsaturated cationic lipid comprises at least two lipid moieties each independently having at least two or at least three sites of unsaturation. In certain instances, at least two of the lipid moieties are independently selected from the group consisting of a dodecadienyl moiety, a tetradecadienyl moiety, a hexadecadienyl moiety, an octadecadienyl moiety, an icosadienyl moiety, a dodecatrienyl moiety, a tetradectrienyl moiety, a hexadecatrienyl moiety, an octadecatrienyl moiety, an icosatrienyl moiety, an arachidonyl moiety, a docosahexaenoyl moiety, and combinations thereof. In preferred embodiments, at least two of the polyunsaturated lipid moieties independently comprise an octadecadienyl moiety (e.g., a linoleyl moiety), an octadecatrienyl moiety (e.g., a linolenyl moiety or a γ-linolenyl moiety), and combinations thereof. In particular embodiments, when the polyunsaturated cationic lipid contains two lipid moieties, both of the lipid moieties are either linoleyl moieties, linolenyl moieties, or γ-linolenyl moieties.

In further embodiments, the polyunsaturated cationic lipid comprises at least three lipid moieties each independently having at least two or at least three sites of unsaturation. In some instances, the polyunsaturated cationic lipid comprises three lipid moieties, and all three lipid moieties are linoleyl moieties, linolenyl moieties, γ-linolenyl moieties, or combinations of these moieties. In further embodiments, the polyunsaturated cationic lipid comprises two, three, or more lipid moieties, wherein at least two of the lipid moieties are different in length, i.e., the polyunsaturated cationic lipid is an asymmetric lipid.

In certain embodiments, the polyunsaturated cationic lipid comprises one or more of the polyunsaturated cationic lipids set forth in Formulas I-XVIX. In preferred embodiments, the polyunsaturated cationic lipid comprises one or more of the following: 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28,31-tetraen-19-yl 4-(dimethylamino) butanoate (DLin-M-C3-DMA or MC3), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), and mixtures thereof.

In particularly preferred embodiments, the nucleic acid-lipid particle composition of the present invention comprises:
(a) a plurality of nucleic acid-lipid particles comprising: a nucleic acid, a polyunsaturated cationic lipid, a non-cationic lipid, and a conjugated lipid that inhibits aggregation of the particle, wherein the polyunsaturated cationic lipid comprises at least one linoleyl moiety, linolenyl moiety, γ-linolenyl moiety, or mixtures thereof; and
(b) an antioxidant, wherein the antioxidant comprises EDTA or a salt thereof, and wherein the antioxidant optionally further comprises at least one additional antioxidant such as, e.g., one or more primary antioxidants, one or more secondary antioxidants, salts thereof, or mixtures thereof. In particular embodiments, the antioxidant comprises a mixture of a primary antioxidant such as α-tocopherol (or a salt thereof) and a secondary antioxidant such as ascorbyl palmitate (or a salt thereof) in combination with EDTA (or a salt thereof).

The non-cationic lipid in the nucleic acid-lipid particles of the invention (e.g., SNALP) may comprise, e.g., one or more anionic lipids and/or neutral lipids. In some embodiments, the non-cationic lipid comprises one of the following neutral lipid components: (1) a mixture of a phospholipid and cholesterol or a derivative thereof; (2) cholesterol or a derivative thereof; or (3) a phospholipid. In certain preferred embodiments, the phospholipid comprises dipalmitoylphosphatidylcholine (DPPC), distearoylphosphatidylcholine (DSPC), or a mixture thereof. In a particularly preferred embodiment, the non-cationic lipid is a mixture of DPPC and cholesterol.

The lipid conjugate in the nucleic acid-lipid particles of the invention (e.g., SNALP) inhibits aggregation of particles and may comprise, e.g., one or more of the lipid conjugates described herein. In one particular embodiment, the lipid conjugate comprises a PEG-lipid conjugate. Examples of PEG-lipid conjugates include, but are not limited to, PEG-DAG conjugates, PEG-DAA conjugates, PEG-cholesterol conjugates, and mixtures thereof. In certain embodiments, the PEG-DAA conjugate in the lipid particle may comprise a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, a PEG-distearyloxypropyl (C₁₈) conjugate, or mixtures thereof. In another embodiment, the lipid conjugate comprises a POZ-lipid conjugate such as a POZ-DAA conjugate.

In some embodiments, the nucleic acid-lipid particles (e.g., SNALP) present in the compositions and methods of the invention comprise: (a) one or more (e.g., a cocktail) of the nucleic acid molecules described herein (e.g., interfering RNAs such as siRNAs); (b) one or more polyunsaturated cationic lipids or salts thereof comprising from about 50 mol % to about 85 mol % of the total lipid present in the particle; (c) one or more non-cationic lipids comprising from about 13 mol % to about 49.5 mol % of the total lipid present in the particle; and (d) one or more conjugated lipids that inhibit aggregation of particles comprising from about 0.5 mol % to about 2 mol % of the total lipid present in the particle.

In one aspect of this embodiment, the nucleic acid-lipid particle comprises: (a) one or more (e.g., a cocktail) of the nucleic acid molecules described herein (e.g., interfering RNAs such as siRNAs); (b) one or more polyunsaturated cationic lipids or salts thereof comprising from about 52 mol % to about 62 mol % of the total lipid present in the particle; (c) a mixture of a phospholipid and cholesterol or a derivative thereof comprising from about 36 mol % to about 47 mol % of the total lipid present in the particle; and (d) a PEG-lipid conjugate comprising from about 1 mol % to about 2 mol % of the total lipid present in the particle. This embodiment of nucleic acid-lipid particle is generally referred to herein as the “1:57” formulation. In certain instances, the non-cationic lipid mixture in the 1:57 formulation comprises: (i) a phospholipid of from about 4 mol % to about 10 mol % of the total lipid present in the particle; and (ii) cholesterol or a derivative thereof of from about 30 mol % to about 40 mol % of the total lipid present in the particle. In one particular embodiment, the 1:57 formulation is a four-component system comprising about 1.4 mol % PEG-lipid conjugate (e.g., PEG2000-C-DMA), about 57.1 mol % cationic lipid (e.g., cationic lipid of Formula I-XVIX) or a salt thereof, about 7.1 mol % DPPC (or DSPC), and about 34.3 mol % cholesterol (or derivative thereof).

In another aspect of this embodiment, the nucleic acid-lipid particle comprises: (a) one or more (e.g., a cocktail) of the nucleic acid molecules described herein (e.g., interfering RNAs such as siRNAs); (b) one or more polyunsaturated cationic lipids or salts thereof comprising from about 56.5 mol % to about 66.5 mol % of the total lipid present in the particle; (c) cholesterol or a derivative thereof comprising from about 31.5 mol % to about 42.5 mol % of the total lipid present in the particle; and (d) a PEG-lipid conjugate comprising from about 1 mol % to about 2 mol % of the total lipid present in the particle. This embodiment of nucleic acid-lipid particle is generally referred to herein as the “1:62” formulation. In one particular embodiment, the 1:62 formulation is a three-component system which is phospholipid-free and comprises about 1.5 mol % PEG-lipid conjugate (e.g., PEG2000-C-DMA), about 61.5 mol % cationic lipid (e.g., cationic lipid of Formula I-XVIX) or a salt thereof, and about 36.9 mol % cholesterol (or derivative thereof).

Additional embodiments related to the 1:57 and 1:62 formulations are described in PCT Publication No. WO 09/127,060 and U.S. application Ser. No. 12/794,701, filed Jun. 4, 2010, the disclosures of which are herein incorporated by reference in their entirety for all purposes.

In other embodiments, the nucleic acid-lipid particles (e.g., SNALP) present in the compositions and methods of the invention comprise: (a) one or more (e.g., a cocktail) of the nucleic acid molecules described herein (e.g., interfering RNAs such as siRNAs); (b) one or more polyunsaturated cationic lipids or salts thereof comprising from about 2 mol % to about 50 mol % of the total lipid present in the particle; (c) one or more non-cationic lipids comprising from about 5 mol % to about 90 mol % of the total lipid present in the particle; and (d) one or more conjugated lipids that inhibit aggregation of particles comprising from about 0.5 mol % to about 20 mol % of the total lipid present in the particle.

In one aspect of this embodiment, the nucleic acid-lipid particle comprises: (a) one or more (e.g., a cocktail) of the nucleic acid molecules described herein (e.g., interfering RNAs such as siRNAs); (b) one or more polyunsaturated cationic lipids or salts thereof comprising from about 30 mol % to about 50 mol % of the total lipid present in the particle; (c) a mixture of a phospholipid and cholesterol or a derivative thereof comprising from about 47 mol % to about 69 mol % of the total lipid present in the particle; and (d) a PEG-lipid conjugate comprising from about 1 mol % to about 3 mol % of the total lipid present in the particle. This embodiment of nucleic acid-lipid particle is generally referred to herein as the “2:40” formulation. In one particular embodiment, the 2:40 formulation is a four-component system which comprises about 2 mol % PEG-lipid conjugate (e.g., PEG2000-C-DMA), about 40 mol % cationic lipid (e.g., cationic lipid of Formula I-XVIX) or a salt thereof, about 10 mol % DPPC (or DSPC), and about 48 mol % cholesterol (or derivative thereof).

In further embodiments, the nucleic acid-lipid particles (e.g., SNALP) present in the compositions and methods of the invention comprise: (a) one or more (e.g., a cocktail) of the nucleic acid molecules described herein (e.g., interfering RNAs such as siRNAs); (b) one or more polyunsaturated cationic lipids or salts thereof comprising from about 50 mol % to about 65 mol % of the total lipid present in the particle; (c) one or more non-cationic lipids comprising from about 25 mol % to about 45 mol % of the total lipid present in the particle; and (d) one or more conjugated lipids that inhibit aggregation of particles comprising from about 5 mol % to about 10 mol % of the total lipid present in the particle.

In one aspect of this embodiment, the nucleic acid-lipid particle comprises: (a) one or more (e.g., a cocktail) of the nucleic acid molecules described herein (e.g., interfering RNAs such as siRNAs); (b) one or more polyunsaturated cationic lipids or salts thereof comprising from about 50 mol % to about 60 mol % of the total lipid present in the particle; (c) a mixture of a phospholipid and cholesterol or a derivative thereof comprising from about 35 mol % to about 45 mol % of the total lipid present in the particle; and (d) a PEG-lipid conjugate comprising from about 5 mol % to about 10 mol % of the total lipid present in the particle. This embodiment of nucleic acid-lipid particle is generally referred to herein as the “7:54” formulation. In certain instances, the non-cationic lipid mixture in the 7:54 formulation comprises: (i) a phospholipid of from about 5 mol % to about 10 mol % of the total lipid present in the particle; and (ii) cholesterol or a derivative thereof of from about 25 mol % to about 35 mol % of the total lipid present in the particle. In one particular embodiment, the 7:54 formulation is a four-component system which comprises about 7 mol % PEG-lipid conjugate (e.g., PEG750-C-DMA), about 54 mol % cationic lipid (e.g., cationic lipid of Formula I-XVIX) or a salt thereof, about 7 mol % DPPC (or DSPC), and about 32 mol % cholesterol (or derivative thereof).

In another aspect of this embodiment, the nucleic acid-lipid particle comprises: (a) one or more (e.g., a cocktail) of the nucleic acid molecules described herein (e.g., interfering RNAs such as siRNAs); (b) one or more polyunsaturated cationic lipids or salts thereof comprising from about 55 mol % to about 65 mol % of the total lipid present in the particle; (c) cholesterol or a derivative thereof comprising from about 30 mol % to about 40 mol % of the total lipid present in the particle; and (d) a PEG-lipid conjugate comprising from about 5 mol % to about 10 mol % of the total lipid present in the particle. This embodiment of nucleic acid-lipid particle is generally referred to herein as the “7:58” formulation. In one particular embodiment, the 7:58 formulation is a three-component system which is phospholipid-free and comprises about 7 mol % PEG-lipid conjugate (e.g., PEG750-C-DMA), about 58 mol % cationic lipid (e.g., cationic lipid of Formula I-XVIX) or a salt thereof, and about 35 mol % cholesterol (or derivative thereof).

Additional embodiments related to the 7:54 and 7:58 formulations are described in U.S. application Ser. No. 12/828,189, filed Jun. 30, 2010, the disclosure of which is herein incorporated by reference in its entirety for all purposes.

The present invention also provides pharmaceutical compositions comprising a nucleic acid-lipid particle such as SNALP, one or more (e.g., a mixture of two, three, or more) antioxidants, and a pharmaceutically acceptable carrier.

In certain embodiments, the nucleic acid component of the nucleic acid-lipid particle (e.g., SNALP) comprises an interfering RNA molecule such as, e.g., an siRNA, aiRNA, miRNA, Dicer-substrate dsRNA, shRNA, ssRNAi oligonucleotides, or mixtures thereof. In other embodiments, the nucleic acid comprises single-stranded or double-stranded DNA, RNA, or a DNA/RNA hybrid such as, e.g., an antisense oligonucleotide, a DNAi oligonucleotide, a ribozyme, an aptamer, a plasmid, an immunostimulatory oligonucleotide, or mixtures thereof. In preferred embodiments, the nucleic acid comprises an siRNA.

In some embodiments, the nucleic acid (e.g., interfering RNA such as siRNA) comprises one, two, three, four, five, six, seven, eight, nine, ten, eleven, twelve, or more modified nucleotides including, but not limited to, 2′-O-methyl (2′OMe) nucleotides, 2′-deoxy-2′-fluoro (2′F) nucleotides, 2′-deoxy nucleotides, 2′-O-(2-methoxyethyl) (MOE) nucleotides, locked nucleic acid (LNA) nucleotides, and mixtures thereof. In preferred embodiments, the nucleic acid (e.g., interfering RNA such as siRNA) comprises one or more 2′OMe nucleotides (e.g., 2′OMe purine and/or pyrimidine nucleotides) such as, e.g., 2′OMe-guanosine nucleotides, 2′OMe-uridine nucleotides, 2′OMe-adenosine nucleotides, 2′OMe-cytosine nucleotides, or mixtures thereof. In one particular embodiment, the nucleic acid (e.g., interfering RNA such as siRNA) comprises at least one 2′OMe-guanosine nucleotide, 2′OMe-uridine nucleotide, or mixtures thereof. In certain instances, the nucleic acid (e.g., interfering RNA such as siRNA) does not comprise 2′OMe-cytosine nucleotides. In other embodiments, the nucleic acid (e.g., interfering RNA such as siRNA) comprises a hairpin loop structure.

In some embodiments, the nucleic acid (e.g., interfering RNA such as siRNA) does not comprise phosphate backbone modifications, e.g., in the sense and/or antisense strand of the double-stranded region of an siRNA. In other embodiments, the nucleic acid (e.g., interfering RNA such as siRNA) comprises one, two, three, four, or more phosphate backbone modifications, e.g., in the sense and/or antisense strand of the double-stranded region of an siRNA. In preferred embodiments, the siRNA does not comprise phosphate backbone modifications.

In further embodiments, the nucleic acid (e.g., interfering RNA such as siRNA) does not comprise 2′-deoxy nucleotides, e.g., in the sense and/or antisense strand of the double-stranded region of an siRNA. In yet further embodiments, the nucleic acid (e.g., interfering RNA such as siRNA) comprises one, two, three, four, or more 2′-deoxy nucleotides, e.g., in the sense and/or antisense strand of the double-stranded region of an siRNA. In preferred embodiments, the siRNA does not comprise 2′-deoxy nucleotides.

In certain instances, the nucleotide at the 3′-end of the double-stranded region in the sense and/or antisense strand of an interfering RNA such as an siRNA is not a modified nucleotide. In certain other instances, the nucleotides near the 3′-end (e.g., within one, two, three, or four nucleotides of the 3′-end) of the double-stranded region in the sense and/or antisense strand of an interfering RNA such as an siRNA are not modified nucleotides.

The interfering RNA (e.g., siRNA) molecules described herein may have 3′ overhangs of one, two, three, four, or more nucleotides on one or both sides of the double-stranded region, or may lack overhangs (i.e., have blunt ends) on one or both sides of the double-stranded region. In certain embodiments, the 3′ overhang on the sense and/or antisense strand of an interfering RNA (e.g., siRNA) independently comprises one, two, three, four, or more modified nucleotides such as 2′OMe nucleotides and/or any other modified nucleotide described herein or known in the art.

In other embodiments, the nucleic acid (e.g., interfering RNA) is fully encapsulated in the nucleic acid-lipid particle (e.g., SNALP). With respect to formulations comprising an siRNA cocktail, the different types of siRNA species present in the cocktail (e.g., siRNA compounds with different sequences) may be co-encapsulated in the same particle, or each type of siRNA species present in the cocktail may be encapsulated in a separate particle. The siRNA cocktail may be formulated in the particles described herein using a mixture of two or more individual siRNAs (each having a unique sequence) at identical, similar, or different concentrations or molar ratios. In one embodiment, a cocktail of siRNAs (corresponding to a plurality of siRNAs with different sequences) is formulated using identical, similar, or different concentrations or molar ratios of each siRNA species, and the different types of siRNAs are co-encapsulated in the same particle. In another embodiment, each type of siRNA species present in the cocktail is encapsulated in different particles at identical, similar, or different siRNA concentrations or molar ratios, and the particles thus formed (each containing a different siRNA payload) are administered separately (e.g., at different times in accordance with a therapeutic regimen), or are combined and administered together as a single unit dose (e.g., with a pharmaceutically acceptable carrier).

In some embodiments, the antioxidant or mixtures thereof prevents, decreases, or inhibits the degradation of the cationic lipid (e.g., polyunsaturated cationic lipid) component of the lipid particle (e.g., nucleic acid-lipid particle) such that the cationic lipid concentration is at least about 50%, 55%, 60%, 65%, 70%, 75%, 80%, 85%, 90%, 91%, 92%, 93%, 94%, 95%, 96%, 97%, 98%, 99%, or up to about 100% of the input cationic lipid concentration, e.g., after about 1, 2, 3, 4, or more weeks and/or after about 1, 2, 3, 4, 5, 6, 7, 8, 9, or more months at 4° C., 5° C., room temperature (RT), 37° C., and/or 40° C. In other embodiments, the antioxidant or mixtures thereof prevents, decreases, or inhibits the degradation of the nucleic acid (e.g., siRNA) payload such that the nucleic acid concentration is at least about 50%, 55%, 60%, 65%, 70%, 75%, 80%, 85%, 90%, 91%, 92%, 93%, 94%, 95%, 96%, 97%, 98%, 99%, or up to about 100% of the input nucleic acid (e.g., siRNA) concentration, e.g., after about 1, 2, 3, 4, or more weeks and/or after about 1, 2, 3, 4, 5, 6, 7, 8, 9, or more months at 4° C., 5° C., room temperature (RT), 37° C., and/or 40° C. Cationic lipid and/or nucleic acid stability can be measured and compared with respect to any length of time (e.g., minutes, hours, days, weeks, months, years, etc.) and at any temperature (e.g., 4° C., 5° C., RT, 37° C., 40° C., etc.). The concentration of cationic lipid and/or nucleic acid present in a nucleic acid-lipid particle over time can be measured by HPLC or any other technique known to one of skill in the art.

In some embodiments, the antioxidant(s) present in the compositions and methods of the invention prevents, decreases, or inhibits the oxidation of the polyunsaturated cationic lipid. In other embodiments, the antioxidant(s) present in the compositions and methods of the invention prevents, decreases, or inhibits the degradation of the nucleic acid payload by preventing, decreasing, or inhibiting the degradation of the polyunsaturated cationic lipid. In further embodiments, the antioxidant(s) present in the compositions and methods of the invention prevents, decreases, or inhibits the desulfurization of a nucleic acid payload comprising one or more phosphorothioate linkages by preventing, decreasing, or inhibiting the degradation of the polyunsaturated cationic lipid.

In some embodiments, the antioxidant or mixtures thereof increases the stability of the lipid particle (e.g., nucleic acid-lipid particle) such that the particle size is less than about 100 nm (e.g., less than about 95 nm, 90 nm, 85 nm, 80 nm, 75 nm, 70 nm, or between about 70 nm to about 100 nm or between about 70 nm to about 90 nm), e.g., after about 1, 2, 3, 4, or more weeks and/or after about 1, 2, 3, 4, 5, 6, 7, 8, 9, or more months at 4° C., 5° C., room temperature (RT), 37° C., and/or 40° C. In other embodiments, the antioxidant or mixtures thereof increases the stability of the lipid particle (e.g., nucleic acid-lipid particle) such that the encapsulation efficiency is greater than about 90% (e.g., greater than about 91%, 92%, 93%, 94%, 95%, 96%, 97%, 98%, 99%, or up to about 100%, or between about 90% to about 100% or between about 95% to about 100%), e.g., after about 1, 2, 3, 4, or more weeks and/or after about 1, 2, 3, 4, 5, 6, 7, 8, 9, or more months at 4° C., 5° C., room temperature (RT), 37° C., and/or 40° C. One skilled in the art will understand that particle size and encapsulation efficiency can be measured and compared with respect to any length of time (e.g., minutes, hours, days, weeks, months, years, etc.) and at any temperature (e.g., 4° C., 5° C., RT, 37° C., 40° C., etc.). As non-limiting examples, analytical assays such as Malvern Nano Series Zetasizer for particle size and Varian Cary Eclipse Fluorimeter for RiboGreen analysis of encapsulation efficiency can be performed on nucleic acid-lipid particles such as SNALP to determine their stability at t=0 and upon storage at one or more temperatures such as 4° C., 5° C., RT, 37° C., and/or 40° C. for about 2 weeks, for about 1 month, and/or for longer.

The compositions and methods of the invention are useful for the therapeutic delivery of nucleic acid molecules (e.g., interfering RNA such as siRNA) that silence the expression of one or more genes. In some embodiments, a cocktail of interfering RNA (e.g., siRNA) is formulated into the same or different nucleic acid-lipid particles, and the particles are administered to a mammal (e.g., a human) requiring such treatment. In certain instances, a therapeutically effective amount of the nucleic acid-lipid particles can be administered to the mammal, e.g., for treating a disease or disorder.

In some embodiments, the nucleic acid-lipid particles described herein (e.g., SNALP) are administered by one of the following routes of administration: oral, intranasal, intravenous, intraperitoneal, intramuscular, intra-articular, intralesional, intratracheal, subcutaneous, and intradermal.

In yet another aspect, the present invention provides methods for introducing one or more nucleic acid molecules (e.g., interfering RNA such as siRNA) into a cell, the method comprising contacting the cell with a nucleic acid-lipid particle (e.g., a SNALP formulation comprising one or more antioxidants). In one particular embodiment, the cell is a liver cell such as, e.g., a hepatocyte present in the liver of a mammal (e.g., a human). In another particular embodiment, the cell is a tumor cell such as, e.g., a cell present in a solid tumor of a mammal (e.g., a human). In some instances, the solid tumor is a liver tumor (e.g., hepatocellular carcinoma). In other instances, the solid tumor is located outside of the liver. In certain embodiments, the cell is a non-tumor cell present in a mammal that produces one or more angiogenic and/or growth factors associated with cell proliferation, tumorigenesis, or cell transformation.

In yet another aspect, the present invention provides methods for the in vivo delivery of one or more nucleic acid molecules (e.g., interfering RNA such as siRNA), the method comprising administering to a mammal (e.g., human) a nucleic acid-lipid particle (e.g., a SNALP formulation comprising one or more antioxidants).

In a related aspect, the present invention provides methods for treating a disease or disorder in a mammal (e.g., human) in need thereof, the method comprising administering to the mammal a therapeutically effective amount of a nucleic acid-lipid particle (e.g., a SNALP formulation comprising one or more antioxidants) comprising one or more nucleic acid molecules (e.g., interfering RNA such as siRNA).

In particular embodiments, the nucleic acid-lipid particles (e.g., SNALP) of the invention can preferentially deliver a payload such as a nucleic acid (e.g., interfering RNA such as siRNA) to the liver as compared to other tissues, e.g., for the treatment of a metabolic disease or disorder such as dyslipidemia. In other particular embodiments, the nucleic acid-lipid particles (e.g., SNALP) of the invention can preferentially deliver a payload such as a nucleic acid (e.g., interfering RNA such as siRNA) to solid tumors as compared to other tissues, e.g., for the treatment of cancer.

In certain instances, a subsequent dose of a nucleic acid-lipid particle formulation described herein (e.g., a SNALP formulation comprising one or more antioxidants) can be administered about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, or 24 hours, or about 1, 2, 3, 4, 5, 6, 7, 8, 9, 10, 11, 12, 13, or 14 days, or about 1, 2, 3, 4, 5, 6, 7, 8, 9, or 10 weeks, or about 1, 2, 3, 4, 5, or 6 months, or any interval thereof, after the initial dose of the same or different nucleic acid-lipid particle formulation. In one particular embodiment, more than one dose of nucleic acid-lipid particles containing one or a cocktail of nucleic acid molecules (e.g., interfering RNA such as siRNA) can be administered at different times in accordance with a therapeutic regimen. In certain instances, a mammal (e.g., human) diagnosed with a disease or disorder can be treated with a second, third, fourth, fifth, sixth, seventh, eighth, ninth, tenth, or more dose of the same or different nucleic acid-lipid particles containing one or a cocktail of nucleic acid molecules (e.g., interfering RNA such as siRNA). In another embodiment, a mammal (e.g., human) diagnosed with a disease or disorder can be treated with a daily dose of the same or different particles containing one or a cocktail of nucleic acid molecules (e.g., interfering RNA such as siRNA) and assessed for a reduction in the severity of clinical symptoms associated with the disease or disorder. In some embodiments, a mammal (e.g., human) susceptible to developing a particular disease or disorder may be pretreated with one or more doses of nucleic acid-lipid particles containing one or a cocktail of nucleic acid molecules (e.g., interfering RNA such as siRNA) as a prophylactic measure for preventing the disease or disorder.

### Lipid Particles

The lipid particles of the invention typically comprise an active agent or therapeutic agent, a cationic lipid, a non-cationic lipid, and a conjugated lipid that inhibits aggregation of particles. In some embodiments, the active agent or therapeutic agent is fully encapsulated within the lipid portion of the lipid particle such that the active agent or therapeutic agent in the lipid particle is resistant in aqueous solution to enzymatic degradation, e.g., by a nuclease or protease. In other embodiments, the lipid particles described herein are substantially non-toxic to mammals such as humans. The lipid particles of the invention typically have a mean diameter of from about 30 nm to about 150 nm, from about 40 nm to about 150 nm, from about 50 nm to about 150 nm, from about 60 nm to about 130 nm, from about 70 nm to about 110 nm, or from about 70 to about 90 nm. The lipid particles of the invention also typically have a lipid:therapeutic agent (e.g., lipid:nucleic acid) ratio (mass/mass ratio) of from about 1:1 to about 100:1, from about 1:1 to about 50:1, from about 2:1 to about 25:1, from about 3:1 to about 20:1, from about 5:1 to about 15:1, or from about 5:1 to about 10:1.

Lipid particles include, but are not limited to, lipid vesicles such as liposomes. As used herein, a lipid vesicle includes a structure having lipid-containing membranes enclosing an aqueous interior. In particular embodiments, lipid vesicles comprising one or more of the cationic lipids described herein are used to encapsulate nucleic acids within the lipid vesicles. In other embodiments, lipid vesicles comprising one or more of the cationic lipids described herein are complexed with nucleic acids to form lipoplexes.

In preferred embodiments, the lipid particles of the invention are serum-stable nucleic acid-lipid particles (SNALP) which comprise an interfering RNA (e.g., dsRNA such as siRNA, Dicer-substrate dsRNA, shRNA, aiRNA, and/or miRNA), a cationic lipid (e.g., one or more polyunsaturated cationic lipids or salts thereof as set forth herein), a non-cationic lipid (e.g., mixtures of one or more phospholipids and cholesterol), and a conjugated lipid that inhibits aggregation of the particles (e.g., one or more PEG-lipid conjugates). The SNALP may comprise at least 1, 2, 3, 4, 5, 6, 7, 8, 9, 10, or more unmodified and/or modified interfering RNA (e.g., siRNA) that target one or more of the genes described herein. Nucleic acid-lipid particles and their method of preparation are described in, e.g., U.S. Pat. Nos. 5,753,613; 5,785,992; 5,705,385; 5,976,567; 5,981,501; 6,110,745; and 6,320,017; and PCT Publication No. WO 96/40964, the disclosures of which are each herein incorporated by reference in their entirety for all purposes.

In the nucleic acid-lipid particles of the invention, the nucleic acid may be fully encapsulated within the lipid portion of the particle, thereby protecting the nucleic acid from nuclease degradation. In preferred embodiments, a SNALP comprising a nucleic acid such as an interfering RNA is fully encapsulated within the lipid portion of the particle, thereby protecting the nucleic acid from nuclease degradation. In certain instances, the nucleic acid in the SNALP is not substantially degraded after exposure of the particle to a nuclease at 37° C. for at least about 20, 30, 45, or 60 minutes. In certain other instances, the nucleic acid in the SNALP is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours. In other embodiments, the nucleic acid is complexed with the lipid portion of the particle. One of the benefits of the formulations of the present invention is that the nucleic acid-lipid particle compositions are substantially non-toxic to mammals such as humans.

The term “fully encapsulated” indicates that the nucleic acid in the nucleic acid-lipid particle is not significantly degraded after exposure to serum or a nuclease assay that would significantly degrade free DNA or RNA. In a fully encapsulated system, preferably less than about 25% of the nucleic acid in the particle is degraded in a treatment that would normally degrade 100% of free nucleic acid, more preferably less than about 10%, and most preferably less than about 5% of the nucleic acid in the particle is degraded. “Fully encapsulated” also indicates that the nucleic acid-lipid particles are serum-stable, that is, that they do not rapidly decompose into their component parts upon in vivo administration.

In the context of nucleic acids, full encapsulation may be determined by performing a membrane-impermeable fluorescent dye exclusion assay, which uses a dye that has enhanced fluorescence when associated with nucleic acid. Specific dyes such as OliGreen® and RiboGreen® (Invitrogen Corp.; Carlsbad, Calif.) are available for the quantitative determination of plasmid DNA, single-stranded deoxyribonucleotides, and/or single- or double-stranded ribonucleotides. Encapsulation is determined by adding the dye to a liposomal formulation, measuring the resulting fluorescence, and comparing it to the fluorescence observed upon addition of a small amount of nonionic detergent. Detergent-mediated disruption of the liposomal bilayer releases the encapsulated nucleic acid, allowing it to interact with the membrane-impermeable dye. Nucleic acid encapsulation may be calculated as E=(Iₒ−I)/Iₒ, where I and Iₒ refer to the fluorescence intensities before and after the addition of detergent (see, Wheeler et al., Gene Ther., 6:271-281 (1999)).

In other embodiments, the present invention provides a nucleic acid-lipid particle (e.g., SNALP) composition comprising a plurality of nucleic acid-lipid particles.

In some instances, the SNALP composition comprises nucleic acid that is fully encapsulated within the lipid portion of the particles, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, or 99% (or any fraction thereof or range therein) of the particles have the nucleic acid encapsulated therein.

In other instances, the SNALP composition comprises nucleic acid that is fully encapsulated within the lipid portion of the particles, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, or 99% (or any fraction thereof or range therein) of the input nucleic acid is encapsulated in the particles.

Depending on the intended use of the lipid particles of the invention, the proportions of the components can be varied and the delivery efficiency of a particular formulation can be measured using, e.g., an endosomal release parameter (ERP) assay.

In one aspect, the lipid particles of the invention may include a targeting lipid. In some embodiments, the targeting lipid comprises a GalNAc moiety (i.e., an N-galactosamine moiety). As a non-limiting example, a targeting lipid comprising a GalNAc moiety can include those described in U.S. application Ser. No. 12/328,669, filed Dec. 4, 2008, the disclosure of which is herein incorporated by reference in its entirety for all purposes. A targeting lipid can also include any other lipid (e.g., targeting lipid) known in the art, for example, as described in U.S. application Ser. No. 12/328,669 or PCT Publication No. WO 2008/042973, the contents of each of which are incorporated herein by reference in their entirety for all purposes. In some embodiments, the targeting lipid includes a plurality of GalNAc moieties, e.g., two or three GalNAc moieties. In some embodiments, the targeting lipid contains a plurality, e.g., two or three N-acetylgalactosamine (GalNAc) moieties. In some embodiments, the lipid in the targeting lipid is 1,2-Di-O-hexadecyl-sn-glyceride (i.e., DSG). In some embodiments, the targeting lipid includes a PEG moiety (e.g., a PEG moiety having a molecular weight of at least about 500 Da, such as about 1000 Da, 1500 Da, 2000 Da or greater), for example, the targeting moiety is connected to the lipid via a PEG moiety. Examples of GalNAc targeting lipids include, but are not limited to, (GalNAc)₃-PEG-DSG, (GalNAc)₃-PEG-LCO, and mixtures thereof.

In some embodiments, the targeting lipid includes a folate moiety. For example, a targeting lipid comprising a folate moiety can include those described in U.S. application Ser. No. 12/328,669, the disclosure of which is herein incorporated by reference in its entirety for all purposes. Examples of folate targeting lipids include, but are not limited to, 1,2-distearoyl-sn-glycero-3-phosphoethanolamine-N-[folate(polyethylene glycol)-2000] (ammonium salt) (Folate-PEG-DSPE), Folate-PEG2000-DSG, Folate-PEG3400-DSG, and mixtures thereof.

In another aspect, the lipid particles of the invention may further comprise one or more apolipoproteins. As used herein, the term “apolipoprotein” or “lipoprotein” refers to apolipoproteins known to those of skill in the art and variants and fragments thereof and to apolipoprotein agonists, analogues, or fragments thereof described in, e.g., PCT Publication No. WO 2010/0088537, the disclosure of which is herein incorporated by reference in its entirety for all purposes. Suitable apolipoproteins include, but are not limited to, ApoA-I, ApoA-II, ApoA-IV, ApoA-V, and ApoE (e.g., ApoE2, ApoE3, etc.), and active polymorphic forms, isoforms, variants, and mutants as well as fragments or truncated forms thereof. Isolated ApoE and/or active fragments and polypeptide analogues thereof, including recombinantly produced forms thereof, are described in U.S. Pat. Nos. 5,672,685; 5,525,472; 5,473,039; 5,182,364; 5,177,189; 5,168,045; and 5,116,739, the disclosures of which are herein incorporated by reference in their entirety for all purposes.

A. Active Agents

Active agents (e.g., therapeutic agents) include any molecule or compound capable of exerting a desired effect on a cell, tissue, organ, or subject. Such effects may be, e.g., biological, physiological, and/or cosmetic. Active agents may be any type of molecule or compound including, but not limited to, nucleic acids, peptides, polypeptides, small molecules, and mixtures thereof. Non-limiting examples of nucleic acids include interfering RNA molecules (e.g., dsRNA such as siRNA, Dicer-substrate dsRNA, shRNA, aiRNA, and/or miRNA), antisense oligonucleotides, plasmids, ribozymes, immunostimulatory oligonucleotides, and mixtures thereof. Examples of peptides or polypeptides include, without limitation, antibodies (e.g., polyclonal antibodies, monoclonal antibodies, antibody fragments; humanized antibodies, recombinant antibodies, recombinant human antibodies, and/or Primatized™ antibodies), cytokines, growth factors, apoptotic factors, differentiation-inducing factors, cell-surface receptors and their ligands, hormones, and mixtures thereof. Examples of small molecules include, but are not limited to, small organic molecules or compounds such as any conventional agent or drug known to those of skill in the art.

In some embodiments, the active agent is a therapeutic agent, or a salt or derivative thereof. Therapeutic agent derivatives may be therapeutically active themselves or they may be prodrugs, which become active upon further modification. Thus, in one embodiment, a therapeutic agent derivative retains some or all of the therapeutic activity as compared to the unmodified agent, while in another embodiment, a therapeutic agent derivative is a prodrug that lacks therapeutic activity, but becomes active upon further modification.

In preferred embodiments, the lipid particles described herein are associated with a nucleic acid, resulting in a nucleic acid-lipid particle (e.g., SNALP). Non-limiting exemplary embodiments related to selecting, synthesizing, and modifying nucleic acids such as siRNA, Dicer-substrate dsRNA, shRNA, aiRNA, miRNA, antisense oligonucleotides, ribozymes, and immunostimulatory oligonucleotides are described, for example, in U.S. Patent Publication No. 20070135372; in U.S. application Ser. No. 12/828,189, filed Jun. 30, 2010; and in PCT Publication No. WO 2010/105372, the disclosures of which are each herein incorporated by reference in their entirety for all purposes.

The nucleic acid (e.g., interfering RNA) component of the nucleic acid-lipid particle (e.g., SNALP) can be used to downregulate or silence the translation (i.e., expression) of a gene of interest. Non-limiting examples of genes of interest include genes associated with metabolic diseases and disorders (e.g., liver diseases and disorders), genes associated with cell proliferation, tumorigenesis, and/or cell transformation (e.g., a cell proliferative disorder such as cancer), angiogenic genes, receptor ligand genes, immunomodulator genes (e.g., those associated with inflammatory and autoimmune responses), genes associated with viral infection and survival, and genes associated with neurodegenerative disorders. See, e.g., U.S. application Ser. No. 12/828,189, filed Jun. 30, 2010, for a description of exemplary target genes which may be downregulated or silenced by the nucleic acid (e.g., interfering RNA) of the nucleic acid-lipid particle (e.g., SNALP).

Non-limiting examples of gene sequences associated with tumorigenesis or cell transformation include polo-like kinase 1 (PLK-1), cyclin-dependent kinase 4 (CDK4), COP1, ring-box 1 (RBX1), WEE1, Eg5 (KSP, KIF11), forkhead box M1 (FOXM1), RAM2 (R1, CDCA7L), XIAP, CSN5 (JAB1), and HDAC2. Non-limiting examples of gene sequences associated with metabolic diseases and disorders include apolipoprotein B (APOB), apolipoprotein CIII (APOC3), apolipoprotein E (APOE), proprotein convertase subtilisin/kexin type 9 (PCSK9), diacylglycerol O-acyltransferase type 1 (DGAT1), and diacylglyerol O-acyltransferase type 2 (DGAT2). Non-limiting examples of gene sequences associated with viral infection and survival include host factors such as tissue factor (TF) or nucleic acid sequences from Filoviruses such as Ebola virus and Marburg virus (e.g., VP30, VP35, nucleoprotein (NP), polymerase protein (L-pol), VP40, glycoprotein (GP), and VP24); Arenaviruses such as Lassa virus, Junin virus, Machupo virus, Guanarito virus, and Sabia virus; Hepatitis viruses such as Hepatitis A, B, C, D, and E viruses; Influenza viruses such as Influenza A, B, and C viruses; Human Immunodeficiency Virus (HIV); Herpes viruses; and Human Papilloma Viruses (HPV).

In other embodiments, the active agent associated with the lipid particles of the invention may comprise one or more therapeutic proteins, polypeptides, or small organic molecules or compounds. Non-limiting examples of such therapeutically effective agents or drugs include oncology drugs (e.g., chemotherapy drugs, hormonal therapeutic agents, immunotherapeutic agents, radiotherapeutic agents, etc.), lipid-lowering agents, anti-viral drugs, anti-inflammatory compounds, antidepressants, stimulants, analgesics, antibiotics, birth control medication, antipyretics, vasodilators, anti-angiogenics, cytovascular agents, signal transduction inhibitors, cardiovascular drugs such as anti-arrhythmic agents, hormones, vasoconstrictors, and steroids. These active agents may be administered alone in the lipid particles of the invention, or in combination (e.g., co-administered) with lipid particles of the invention comprising nucleic acid such as interfering RNA. Non-limiting examples of these types of active agents are described, for example, in U.S. application Ser. No. 12/828,189, filed Jun. 30, 2010, the disclosure of which is herein incorporated by reference in its entirety for all purposes.

B. Cationic Lipids

Any of a variety of cationic lipids or salts thereof may be used in the lipid particles of the present invention (e.g., SNALP), either alone or in combination with one or more other cationic lipid species or non-cationic lipid species. In particular embodiments, one or more of the cationic lipids of Formula I-XVIX or salts thereof as set forth herein may be used in the lipid particles of the present invention (e.g., SNALP), either alone or in combination with one or more other cationic lipid species or non-cationic lipid species. The cationic lipids include the (R) and/or (S) enantiomers thereof. In preferred embodiments, the lipid particles of the present invention (e.g., SNALP) comprise at least one polyunsaturated cationic lipid (e.g., at least one, two, three, four, five, or more polyunsaturated cationic lipids).

In some embodiments, the cationic lipid comprises a racemic mixture. In other embodiments, the cationic lipid comprises a mixture of one or more diastereomers. In certain embodiments, the cationic lipid is enriched in one enantiomer, such that the cationic lipid comprises at least about 55%, 60%, 65%, 70%, 75%, 80%, 85%, 90%, or 95% enantiomeric excess. In certain other embodiments, the cationic lipid is enriched in one diastereomer, such that the cationic lipid comprises at least about 55%, 60%, 65%, 70%, 75%, 80%, 85%, 90%, or 95% diastereomeric excess. In certain additional embodiments, the cationic lipid is chirally pure (e.g., comprises a single optical isomer). In further embodiments, the cationic lipid is enriched in one optical isomer (e.g., an optically active isomer), such that the cationic lipid comprises at least about 55%, 60%, 65%, 70%, 75%, 80%, 85%, 90%, or 95% isomeric excess. The present invention provides the synthesis of the cationic lipids of Formulas I-XVIX as a racemic mixture or in optically pure form.

The term “alkyl” includes a straight chain or branched, noncyclic or cyclic, saturated aliphatic hydrocarbon containing from 1 to 24 carbon atoms. Representative saturated straight chain alkyls include, but are not limited to, methyl, ethyl, n-propyl, n-butyl, n-pentyl, n-hexyl, and the like, while saturated branched alkyls include, without limitation, isopropyl, sec-butyl, isobutyl, tert-butyl, isopentyl, and the like. Representative saturated cyclic alkyls include, but are not limited to, cyclopropyl, cyclobutyl, cyclopentyl, cyclohexyl, and the like, while unsaturated cyclic alkyls include, without limitation, cyclopentenyl, cyclohexenyl, and the like.

The term “alkenyl” includes an alkyl, as defined above, containing at least one double bond between adjacent carbon atoms. Alkenyls include both cis and trans isomers. Representative straight chain and branched alkenyls include, but are not limited to, ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like.

The term “alkynyl” includes any alkyl or alkenyl, as defined above, which additionally contains at least one triple bond between adjacent carbons. Representative straight chain and branched alkynyls include, without limitation, acetylenyl, propynyl, 1-butynyl, 2-butynyl, 1-pentynyl, 2-pentynyl, 3-methyl-1 butynyl, and the like.

The term “acyl” includes any alkyl, alkenyl, or alkynyl wherein the carbon at the point of attachment is substituted with an oxo group, as defined below. The following are non-limiting examples of acyl groups: —C(═O)alkyl, —C(═O)alkenyl, and —C(═O)alkynyl.

The term “heterocycle” includes a 5- to 7-membered monocyclic, or 7- to 10-membered bicyclic, heterocyclic ring which is either saturated, unsaturated, or aromatic, and which contains from 1 or 2 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include, but are not limited to, heteroaryls as defined below, as well as morpholinyl, pyrrolidinonyl, pyrrolidinyl, piperidinyl, piperizinyl, hydantoinyl, valerolactamyl, oxiranyl, oxetanyl, tetrahydrofuranyl, tetrahydropyranyl, tetrahydropyridinyl, tetrahydropyrimidinyl, tetrahydrothiophenyl, tetrahydrothiopyranyl, tetrahydropyrimidinyl, tetrahydrothiophenyl, tetrahydrothiopyranyl, and the like.

The terms “optionally substituted alkyl”, “optionally substituted alkenyl”, “optionally substituted alkynyl”, “optionally substituted acyl”, and “optionally substituted heterocycle” mean that, when substituted, at least one hydrogen atom is replaced with a substituent. In the case of an oxo substituent (═O), two hydrogen atoms are replaced. In this regard, substituents include, but are not limited to, oxo, halogen, heterocycle, —CN, —OR^{x}, —NR^{x}R^{y}, —NR^{x}C(═O)R^{y}, —NR^{x}SO₂R^{y}, —C(═O)R^{x}, —C(═O)OR^{x}, —C(═O)NR^{x}R^{y}, —SOₙR^{x}, and —SOₙNR^{x}R^{y}, wherein n is 0, 1, or 2, R^{x} and R^{y} are the same or different and are independently hydrogen, alkyl, or heterocycle, and each of the alkyl and heterocycle substituents may be further substituted with one or more of oxo, halogen, —OH, —CN, alkyl, —OR^{x}, heterocycle, —NR^{x}R^{y}, —NR^{x}C(═O)R^{y}, —NR^{x}SO₂R^{y}, —C(═O)R^{x}, —C(═O)OR^{x}, —C(═O)NR^{x}R^{y}, —SOₙR^{x}, and —SOₙNR^{x}R^{y}. The term “optionally substituted,” when used before a list of substituents, means that each of the substituents in the list may be optionally substituted as described herein.

The term “halogen” includes fluoro, chloro, bromo, and iodo.

In one aspect, cationic lipids of Formula I having the following structure (or salts thereof) are useful in the present invention:

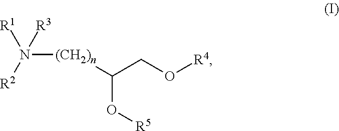

wherein R¹ and R² are either the same or different and are independently hydrogen (H) or an optionally substituted C₁-C₆ alkyl, C₂-C₆ alkenyl, or C₂-C₆ alkynyl, or R¹ and R² may join to form an optionally substituted heterocyclic ring of 4 to 6 carbon atoms and 1 or 2 heteroatoms selected from the group consisting of nitrogen (N), oxygen (O), and mixtures thereof;
R³ is either absent or is hydrogen (H) or a C₁-C₆ alkyl to provide a quaternary amine;
R⁴ and R⁵ are either the same or different and are independently an optionally substituted C₁₀-C₂₄ alkyl, C₁₀-C₂₄ alkenyl, C₁₀-C₂₄ alkynyl, or C₁₀-C₂₄ acyl, wherein at least one of R⁴ and R⁵ comprises at least two sites of unsaturation; and
n is 0, 1, 2, 3, or 4.

In some embodiments, R¹ and R² are independently an optionally substituted C₁-C₄ alkyl, C₂-C₄ alkenyl, or C₂-C₄ alkynyl. In one preferred embodiment, R¹ and R² are both methyl groups. In other preferred embodiments, n is 1 or 2. In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine. In further embodiments, R⁴ and R⁵ are independently an optionally substituted C₁₂-C₂₄, C₁₂-C₂₂, C₁₂-C₂₀, C₁₄-C₂₄, C₁₄-C₂₂, C₁₄-C₂₀, C₁₆-C₂₄, C₁₆-C₂₂, or C₁₆-C₂₀ alkyl, alkenyl, alkynyl, or acyl group (i.e., C₁₂, C₁₃, C₁₄, C₁₅, C₁₆, C₁₇, C₁₈, C₁₉, C₂₀, C₂₁, C₂₂, C₂₃, or C₂₄ alkyl, alkenyl, alkynyl, or acyl group). In certain embodiments, at least one or both R⁴ and R⁵ independently comprises at least 2, 3, 4, 5, or 6 sites of unsaturation (e.g., 2, 3, 4, 5, 6, 2-3, 2-4, 2-5, or 2-6 sites of unsaturation).

In certain instances, R⁴ and R⁵ may independently comprise a dodecadienyl moiety, a tetradecadienyl moiety, a hexadecadienyl moiety, an octadecadienyl moiety, an icosadienyl moiety, a dodecatrienyl moiety, a tetradectrienyl moiety, a hexadecatrienyl moiety, an octadecatrienyl moiety, an icosatrienyl moiety, or an acyl derivative thereof (e.g., linoleoyl, linolenoyl, γ-linolenoyl, etc.). In some instances, the octadecadienyl moiety is a linoleyl moiety. In particular embodiments, R⁴ and R⁵ are both linoleyl moieties. In other instances, the octadecatrienyl moiety is a linolenyl moiety or a γ-linolenyl moiety. In particular embodiments, R⁴ and R⁵ are both linolenyl moieties or γ-linolenyl moieties. In certain instances, R⁴ and R⁵ are different, e.g., R⁴ is a tetradectrienyl (C₁₄) and R⁵ is linoleyl (C₁₈). In a preferred embodiment, the cationic lipid of Formula I is symmetrical, i.e., R⁴ and R⁵ are both the same. In further embodiments, the double bonds present in one or both R⁴ and R⁵ may be in the cis and/or trans configuration.

In some groups of embodiments to the cationic lipids of Formula I, R⁴ and R⁵ are either the same or different and are independently selected from the group consisting of:

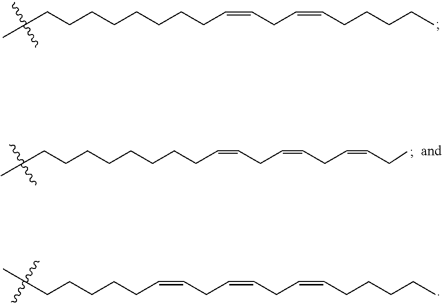 [SMILES: *CCCCCCCC/C=C\C/C=C\CCCCC ; *CCCCCCCC/C=C\C/C=C\C/C=C\CC ; *CCCCC/C=C\C/C=C\C/C=C\CCCCC]

In particular embodiments, the cationic lipid of Formula I comprises 1,2-dilinoleyloxy-N,N-dimethylaminopropane (DLinDMA), 1,2-dilinolenyloxy-N,N-dimethylaminopropane (DLenDMA), or mixtures thereof.

In some embodiments, the cationic lipid of Formula I forms a salt (preferably a crystalline salt) with one or more anions. In one particular embodiment, the cationic lipid of Formula I is the oxalate (e.g., hemioxalate) salt thereof, which is preferably a crystalline salt.

In another aspect, cationic lipids of Formula II having the following structure (or salts thereof) are useful in the present invention:

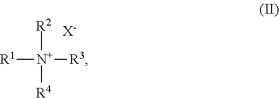

wherein R¹ and R² are independently selected and are H or C₁-C₃ alkyls, R³ and R⁴ are independently selected and are alkyl groups having from about 10 to about 20 carbon atoms, and at least one of R³ and R⁴ comprises at least two sites of unsaturation. In certain instances, R³ and R⁴ are both the same, i.e., R³ and R⁴ are both linoleyl (C₁₈), etc. In certain other instances, R³ and R⁴ are different, i.e., R³ is tetradectrienyl (C₁₄) and R⁴ is linoleyl (C₁₈). In a preferred embodiment, the cationic lipid of Formula II is symmetrical, i.e., R³ and R⁴ are both the same. In another preferred embodiment, both R³ and R⁴ comprise at least two sites of unsaturation. In some embodiments, R³ and R⁴ are independently selected from the group consisting of dodecadienyl, tetradecadienyl, hexadecadienyl, linoleyl, and icosadienyl. In a preferred embodiment, R³ and R⁴ are both linoleyl. In some embodiments, R³ and R⁴ comprise at least three sites of unsaturation and are independently selected from, e.g., dodecatrienyl, tetradectrienyl, hexadecatrienyl, linolenyl, and icosatrienyl.

In some embodiments, the cationic lipid of Formula II forms a salt (preferably a crystalline salt) with one or more anions. In one particular embodiment, the cationic lipid of Formula II is the oxalate (e.g., hemioxalate) salt thereof, which is preferably a crystalline salt.

The synthesis of cationic lipids such as DLinDMA and DLenDMA, as well as additional cationic lipids falling within the scope of Formulas I and II, is described in U.S. Patent Publication No. 20060083780, the disclosure of which is herein incorporated by reference in its entirety for all purposes.

In yet another aspect, cationic lipids of Formula III having the following structure (or salts thereof) are useful in the present invention:

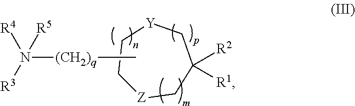

wherein R¹ and R² are either the same or different and are independently an optionally substituted C₁₂-C₂₄ alkyl, C₁₂-C₂₄ alkenyl, C₁₂-C₂₄ alkynyl, or C₁₂-C₂₄ acyl; R³ and R⁴ are either the same or different and are independently an optionally substituted C₁-C₆ alkyl, C₂-C₆ alkenyl, or C₂-C₆ alkynyl, or R³ and R⁴ may join to form an optionally substituted heterocyclic ring of 4 to 6 carbon atoms and 1 or 2 heteroatoms chosen from nitrogen and oxygen; R⁵ is either absent or is hydrogen (H) or a C₁-C₆ alkyl to provide a quaternary amine; m, n, and p are either the same or different and are independently either 0, 1, or 2, with the proviso that m, n, and p are not simultaneously 0; q is 0, 1, 2, 3, or 4; and Y and Z are either the same or different and are independently O, S, or NH.

In some embodiments, R³ and R⁴ are independently an optionally substituted C₁-C₄ alkyl, C₂-C₄ alkenyl, or C₂-C₄ alkynyl. In a preferred embodiment, R³ and R⁴ are both methyl groups. In one embodiment, q is 1 or 2. In another embodiment, q is 1-2, 1-3, 1-4, 2-3, or 2-4. In further embodiments, R⁵ is absent when the pH is above the pKₐ of the cationic lipid and R⁵ is hydrogen when the pH is below the pKₐ of the cationic lipid such that the amino head group is protonated. In an alternative embodiment, R⁵ is an optionally substituted C₁-C₄ alkyl to provide a quaternary amine. In additional embodiments, Y and Z are both O.

In other embodiments, R¹ and R² are independently an optionally substituted C₁₂-C₂₄, C₁₂-C₂₂, C₁₂-C₂₀, C₁₄-C₂₄, C₁₄-C₂₂, C₁₄-C₂₀, C₁₆-C₂₄, C₁₆-C₂₂, or C₁₆-C₂₀ alkyl, alkenyl, alkynyl, or acyl group (i.e., C₁₂, C₁₃, C₁₄, C₁₅, C₁₆, C₁₇, C₁₈, C₁₉, C₂₀, C₂₁, C₂₂, C₂₃, or C₂₄ alkyl, alkenyl, alkynyl, or acyl group). In certain embodiments, at least one or both R¹ and R² independently comprises at least 1, 2, 3, 4, 5, or 6 sites of unsaturation (e.g., 1-2, 1-3, 1-4, 1-5, 1-6, 2-3, 2-4, 2-5, or 2-6 sites of unsaturation) or a substituted alkyl or acyl group. In certain instances, the unsaturated side-chain may comprise a myristoleyl moiety, a palmitoleyl moiety, an oleyl moiety, a dodecadienyl moiety, a tetradecadienyl moiety, a hexadecadienyl moiety, an octadecadienyl moiety, an icosadienyl moiety, a dodecatrienyl moiety, a tetradectrienyl moiety, a hexadecatrienyl moiety, an octadecatrienyl moiety, an icosatrienyl moiety, or an acyl derivative thereof (e.g., linoleoyl, linolenoyl, etc.). In some instances, the octadecadienyl moiety is a linoleyl moiety. In particular embodiments, R¹ and R² are both linoleyl moieties. In other instances, the octadecatrienyl moiety is a linolenyl moiety or a γ-linolenyl moiety. In particular embodiments, R¹ and R² are both linolenyl moieties or γ-linolenyl moieties.

In embodiments where one or both R¹ and R² independently comprises at least 1, 2, 3, 4, 5, or 6 sites of unsaturation, the double bonds present in one or both R¹ and R² may be in the cis and/or trans configuration. In certain instances, R¹ and R² are both the same, e.g., R¹ and R² are both linoleyl (C₁₈) moieties, etc. In certain other instances, R¹ and R² are different, e.g., R′ is a tetradectrienyl (C₁₄) moiety and R² is a linoleyl (C₁₈) moiety. In a preferred embodiment, the cationic lipid of Formula III is symmetrical, i.e., R¹ and R² are both the same. In another preferred embodiment, at least one or both R¹ and R² comprises at least two sites of unsaturation (e.g., 2, 3, 4, 5, 6, 2-3, 2-4, 2-5, or 2-6 sites of unsaturation).

In embodiments where one or both R¹ and R² independently comprises a branched alkyl or acyl group (e.g., a substituted alkyl or acyl group), the branched alkyl or acyl group may comprise a C₁₂-C₂₄ alkyl or acyl having at least 1-6 (e.g., 1, 2, 3, 4, 5, 6, or more) C₁-C₆ alkyl substituents. In particular embodiments, the branched alkyl or acyl group comprises a C₁₂-C₂₀ or C₁₄-C₂₂ alkyl or acyl with 1-6 (e.g., 1, 2, 3, 4, 5, 6) C₁-C₄ alkyl (e.g., methyl, ethyl, propyl, or butyl) substituents. In some embodiments, the branched alkyl group comprises a phytanoyl (3,7,11,15-tetramethyl-hexadecanoyl) moiety and the branched acyl group comprises a phytanoyl (3,7,11,15-tetramethyl-hexadecanoyl) moiety. In particular embodiments, R¹ and R² are both phytanoyl moieties.

In some groups of embodiments to the cationic lipids of Formula III, R¹ and R² are either the same or different and are independently selected from the group consisting of:

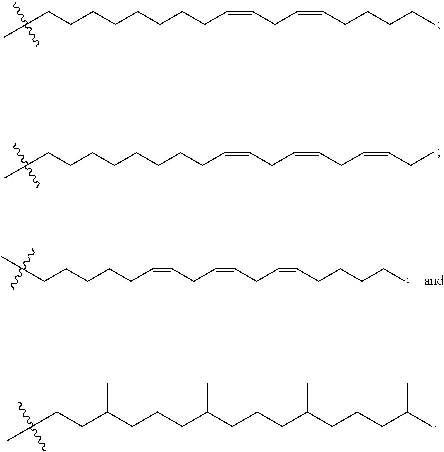 [SMILES: *CCCCCCCC/C=C\C/C=C\CCCCC ; *CCCCCCCC/C=C\C/C=C\C/C=C\CC ; *CCCCC/C=C\C/C=C\C/C=C\CCCCC ; *CCC(C)CCCC(C)CCCC(C)CCCC(C)C]

In certain embodiments, cationic lipids falling within the scope of Formula III include, but are not limited to, the following: 2,2-dilinoleyl-4-(2-dimethylaminoethyl)-[1,3]-dioxolane (DLin-K-C2-DMA; “XTC2” or “C2K”), 2,2-dilinoleyl-4-dimethylaminomethyl-[1,3]-dioxolane (DLin-K-DMA), 2,2-dilinoleyl-4-(3-dimethylaminopropyl)[1,3]-dioxolane (DLin-K-C3-DMA; “C3K”), 2,2-dilinoleyl-4-(4-dimethylaminobutyl)-[1,3]-dioxolane (DLin-K-C4-DMA; “C4K”), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dioleoyl-4-dimethylaminomethyl-[1,3]-dioxolane (DO-K-DMA), 2,2-distearoyl-4-dimethylaminomethyl-[1,3]-dioxolane (DS-K-DMA), 2,2-dilinoleyl-4-N-morpholino-[1,3]-dioxolane (DLin-K-MA), 2,2-Dilinoleyl-4-trimethylamino-[1,3]-dioxolane chloride (DLin-K-TMA.Cl), 2,2-dilinoleyl-4,5-bis(dimethylaminomethyl)-[1,3]-dioxolane (DLin-K²-DMA), 2,2-dilinoleyl-4-methylpiperzine-[1,3]-dioxolane (D-Lin-K-N-methylpiperzine), DLen-C2K-DMA, γ-DLen-C2K-DMA, DPan-C2K-DMA, DPan-C3K-DMA, or mixtures thereof. In preferred embodiments, the cationic lipid of Formula III comprises DLin-K-C2-DMA and/or DLin-K-DMA.

In some embodiments, the cationic lipids of Formula III form a salt (preferably a crystalline salt) with one or more anions. In one particular embodiment, the cationic lipid of Formula III is the oxalate (e.g., hemioxalate) salt thereof, which is preferably a crystalline salt.

The synthesis of cationic lipids such as DLin-K-C2-DMA, DLin-K-C3-DMA, DLin-K-C4-DMA, DLin-K6-DMA, DLin-K-MPZ, DO-K-DMA, DS-K-DMA, DLin-K-MA, DLin-K-TMA.Cl, DLin-K²-DMA, D-Lin-K-N-methylpiperzine, as well as additional cationic lipids, is described in PCT Publication No. WO 2010/042877, the disclosure of which is incorporated herein by reference in its entirety for all purposes.

The synthesis of cationic lipids such as DLin-K-DMA, as well as additional cationic lipids, is described in PCT Publication No. WO 09/086,558, the disclosure of which is herein incorporated by reference in its entirety for all purposes.

In particular embodiments, cationic lipids of Formula IV having the following structure (or salts thereof) are useful in the present invention:

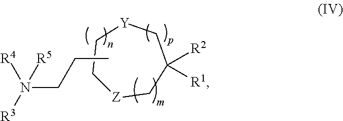

wherein R¹ and R² are either the same or different and are independently an optionally substituted C₁₂-C₂₄ alkyl, C₁₂-C₂₄ alkenyl, C₁₂-C₂₄ alkynyl, or C₁₂-C₂₄ acyl; R³ and R⁴ are either the same or different and are independently an optionally substituted C₁-C₆ alkyl, C₂-C₆ alkenyl, or C₂-C₆ alkynyl, or R³ and R⁴ may join to form an optionally substituted heterocyclic ring of 4 to 6 carbon atoms and 1 or 2 heteroatoms chosen from nitrogen and oxygen; R⁵ is either absent or is hydrogen (H) or a C₁-C₆ alkyl to provide a quaternary amine; m, n, and p are either the same or different and are independently either 0, 1, or 2, with the proviso that m, n, and p are not simultaneously 0; and Y and Z are either the same or different and are independently O, S, or NH.

In some embodiments, R³ and R⁴ are independently an optionally substituted C₁-C₄ alkyl, C₂-C₄ alkenyl, or C₂-C₄ alkynyl. In a preferred embodiment, R³ and R⁴ are both methyl groups. In further embodiments, R⁵ is absent when the pH is above the pKₐ of the cationic lipid and R⁵ is hydrogen when the pH is below the pKₐ of the cationic lipid such that the amino head group is protonated. In an alternative embodiment, R⁵ is an optionally substituted C₁-C₄ alkyl to provide a quaternary amine. In additional embodiments, Y and Z are both O.

In other embodiments, R¹ and R² are independently an optionally substituted C₁₂-C₂₄, C₁₂-C₂₂, C₁₂-C₂₀, C₁₄-C₂₄, C₁₄-C₂₂, C₁₄-C₂₀, C₁₆-C₂₄, C₁₆-C₂₂, or C₁₆-C₂₀ alkyl, alkenyl, alkynyl, or acyl group (i.e., C₁₂, C₁₃, C₁₄, C₁₅, C₁₆, C₁₇, C₁₈, C₁₉, C₂₀, C₂₁, C₂₂, C₂₃, or C₂₄ alkyl, alkenyl, alkynyl, or acyl group). In certain embodiments, at least one or both R¹ and R² independently comprises at least 1, 2, 3, 4, 5, or 6 sites of unsaturation (e.g., 1-2, 1-3, 1-4, 1-5, 1-6, 2-3, 2-4, 2-5, or 2-6 sites of unsaturation) or a substituted alkyl or acyl group. In certain instances, the unsaturated side-chain may comprise a myristoleyl moiety, a palmitoleyl moiety, an oleyl moiety, a dodecadienyl moiety, a tetradecadienyl moiety, a hexadecadienyl moiety, an octadecadienyl moiety, an icosadienyl moiety, a dodecatrienyl moiety, a tetradectrienyl moiety, a hexadecatrienyl moiety, an octadecatrienyl moiety, an icosatrienyl moiety, or an acyl derivative thereof (e.g., linoleoyl, linolenoyl, etc.). In some instances, the octadecadienyl moiety is a linoleyl moiety. In particular embodiments, R¹ and R² are both linoleyl moieties. In other instances, the octadecatrienyl moiety is a linolenyl moiety or a γ-linolenyl moiety. In particular embodiments, R¹ and R² are both linolenyl moieties or γ-linolenyl moieties.

In embodiments where one or both R¹ and R² independently comprises at least 1, 2, 3, 4, 5, or 6 sites of unsaturation, the double bonds present in one or both R¹ and R² may be in the cis and/or trans configuration. In certain instances, R¹ and R² are both the same, e.g., R¹ and R² are both linoleyl (C₁₈) moieties, etc. In certain other instances, R¹ and R² are different, e.g., R¹ is a tetradectrienyl (C₁₄) moiety and R² is a linoleyl (C₁₈) moiety. In a preferred embodiment, the cationic lipid of Formula IV is symmetrical, i.e., R¹ and R² are both the same. In another preferred embodiment, at least one or both R¹ and R² comprises at least two sites of unsaturation (e.g., 2, 3, 4, 5, 6, 2-3, 2-4, 2-5, or 2-6 sites of unsaturation).

In embodiments where one or both R¹ and R² independently comprises a branched alkyl or acyl group (e.g., a substituted alkyl or acyl group), the branched alkyl or acyl group may comprise a C₁₂-C₂₄ alkyl or acyl having at least 1-6 (e.g., 1, 2, 3, 4, 5, 6, or more) C₁-C₆ alkyl substituents. In particular embodiments, the branched alkyl or acyl group comprises a C₁₂-C₂₀ or C₁₄-C₂₂ alkyl or acyl with 1-6 (e.g., 1, 2, 3, 4, 5, 6) C₁-C₄ alkyl (e.g., methyl, ethyl, propyl, or butyl) substituents. In some embodiments, the branched alkyl group comprises a phytanyl (3,7,11,15-tetramethyl-hexadecanyl) moiety and the branched acyl group comprises a phytanoyl (3,7,11,15-tetramethyl-hexadecanoyl) moiety. In particular embodiments, R¹ and R² are both phytanyl moieties.

In some groups of embodiments to the cationic lipids of Formula IV, R¹ and R² are either the same or different and are independently selected from the group consisting of:

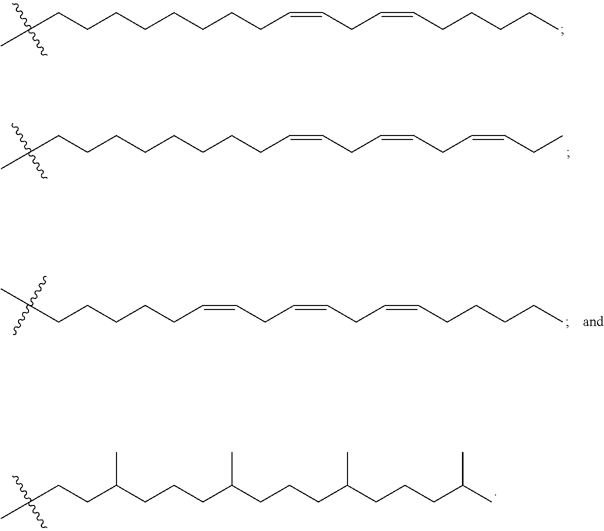 [SMILES: *CCCCCCCC/C=C\C/C=C\CCCCC ; *CCCCCCCC/C=C\C/C=C\C/C=C\CC ; *CCCCC/C=C\C/C=C\C/C=C\CCCCC ; *CCC(C)CCCC(C)CCCC(C)CCCC(C)C]

In certain embodiments, cationic lipids falling within the scope of Formula IV include, but are not limited to, the following: 2,2-dilinoleyl-4-(2-dimethylaminoethyl)-[1,3]-dioxolane (DLin-K-C2-DMA; “XTC2” or “C2K”), DLen-C2K-DMA, γ-DLen-C2K-DMA, DPan-C2K-DMA, or mixtures thereof. In preferred embodiments, the cationic lipid of Formula IV comprises DLin-K-C2-DMA.

In some embodiments, the cationic lipids of Formula IV form a salt (preferably a crystalline salt) with one or more anions. In one particular embodiment, the cationic lipid of Formula IV is the oxalate (e.g., hemioxalate) salt thereof, which is preferably a crystalline salt.

The synthesis of DLin-K-C2-DMA (C2K) is described in PCT Publication No. WO 2010/042877, the disclosure of which is incorporated herein by reference in its entirety for all purposes.

In a further aspect, cationic lipids of Formula V having the following structure are useful in the present invention:

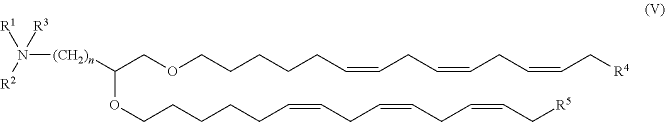

or salts thereof, wherein: R¹ and R² are either the same or different and are independently an optionally substituted C₁-C₆ alkyl, C₂-C₆ alkenyl, or C₂-C₆ alkynyl, or R¹ and R² may join to form an optionally substituted heterocyclic ring of 4 to 6 carbon atoms and 1 or 2 heteroatoms selected from the group consisting of nitrogen (N), oxygen (O), and mixtures thereof; R³ is either absent or is hydrogen (H) or a C₁-C₆ alkyl to provide a quaternary amine; R⁴ and R⁵ are either absent or present and when present are either the same or different and are independently an optionally substituted C₁-C₁₀ alkyl or C₂-C₁₀ alkenyl; and n is 0, 1, 2, 3, or 4.

In some embodiments, R¹ and R² are independently an optionally substituted C₁-C₄ alkyl, C₂-C₄ alkenyl, or C₂-C₄ alkynyl. In a preferred embodiment, R¹ and R² are both methyl groups. In another preferred embodiment, R⁴ and R⁵ are both butyl groups. In yet another preferred embodiment, n is 1. In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine. In further embodiments, R⁴ and R⁵ are independently an optionally substituted C₂-C₆ or C₂-C₄ alkyl or C₂-C₆ or C₂-C₄ alkenyl.

In an alternative embodiment, the cationic lipid of Formula V comprises ester linkages between the amino head group and one or both of the alkyl chains. In some embodiments, the cationic lipid of Formula V forms a salt (preferably a crystalline salt) with one or more anions. In one particular embodiment, the cationic lipid of Formula V is the oxalate (e.g., hemioxalate) salt thereof, which is preferably a crystalline salt.

Although each of the alkyl chains in Formula V contains cis double bonds at positions 6, 9, and 12 (i.e., cis,cis,cis-Δ⁶,Δ⁹,Δ¹²), in an alternative embodiment, one, two, or three of these double bonds in one or both alkyl chains may be in the trans configuration.

In a particularly preferred embodiment, the cationic lipid of Formula V has the structure:

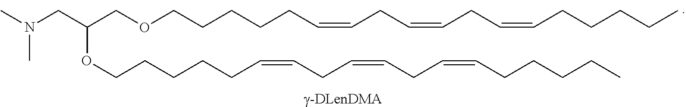 [SMILES: CCCCC/C=C\C/C=C\C/C=C\CCCCCOCC(CN(C)C)OCCCCC/C=C\C/C=C\C/C=C\CCCCC]

In another aspect, cationic lipids of Formula VI having the following structure are useful in the present invention:

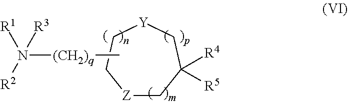

or salts thereof, wherein: R¹ and R² are either the same or different and are independently an optionally substituted C₁-C₆ alkyl, C₂-C₆ alkenyl, or C₂-C₆ alkynyl, or R¹ and R² may join to form an optionally substituted heterocyclic ring of 4 to 6 carbon atoms and 1 or 2 heteroatoms selected from the group consisting of nitrogen (N), oxygen (O), and mixtures thereof; R³ is either absent or is hydrogen (H) or a C₁-C₆ alkyl to provide a quaternary amine; R⁴ and R⁵ are either the same or different and are independently an optionally substituted C₁₂-C₂₄ alkyl, C₁₂-C₂₄ alkenyl, C₁₂-C₂₄ alkynyl, or C₁₂-C₂₄ acyl, wherein at least one of R⁴ and R⁵ comprises at least three sites of unsaturation or a substituted C₁₂-C₂₄ alkyl; m, n, and p are either the same or different and are independently either 0, 1, or 2, with the proviso that m, n, and p are not simultaneously 0; q is 0, 1, 2, 3, or 4; and Y and Z are either the same or different and are independently O, S, or NH.

In some embodiments, R¹ and R² are independently an optionally substituted C₁-C₄ alkyl, C₂-C₄ alkenyl, or C₂-C₄ alkynyl. In a preferred embodiment, R¹ and R² are both methyl groups. In another preferred embodiment, q is 2. In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine. In further embodiments, R⁴ and R⁵ are independently an optionally substituted C₁₂-C₂₀ or C₁₄-C₂₂ alkyl, C₁₂-C₂₀ or C₁₄-C₂₂ alkenyl, C₁₂-C₂₀ or C₁₄-C₂₂ alkynyl, or C₁₂-C₂₀ or C₁₄-C₂₂ acyl.

In embodiments where at least one of R⁴ and R⁵ comprises a branched alkyl group (e.g., a substituted C₁₂-C₂₄ alkyl group), the branched alkyl group may comprise a C₁₂-C₂₄ alkyl having at least 1-6 (e.g., 1, 2, 3, 4, 5, 6, or more) C₁-C₆ alkyl substituents. In particular embodiments, the branched alkyl group comprises a C₁₂-C₂₀ or C₁₄-C₂₂ alkyl with 1-6 (e.g., 1, 2, 3, 4, 5, 6) C₁-C₄ alkyl (e.g., methyl, ethyl, propyl, or butyl) substituents. Preferably, the branched alkyl group comprises a phytanyl (3,7,11,15-tetramethyl-hexadecanyl) moiety. In other preferred embodiments, R⁴ and R⁵ are both phytanyl moieties.

In alternative embodiments, at least one of R⁴ and R⁵ comprises a branched acyl group (e.g., a substituted C₁₂-C₂₄ acyl group). In certain instances, the branched acyl group may comprise a C₁₂-C₂₄ acyl having at least 1-6 (e.g., 1, 2, 3, 4, 5, 6, or more) C₁-C₆ alkyl substituents. In particular embodiments, the branched acyl group comprises a C₁₂-C₂₀ or C₁₄-C₂₂ acyl with 1-6 (e.g., 1, 2, 3, 4, 5, 6) C₁-C₄ alkyl (e.g., methyl, ethyl, propyl, or butyl) substituents. Preferably, the branched acyl group comprises a phytanoyl (3,7,11,15-tetramethyl-hexadecanoyl) moiety.

In embodiments where at least one of R⁴ and R⁵ comprises at least three sites of unsaturation, the double bonds present in one or both alkyl chains may be in the cis and/or trans configuration. In some embodiments, R⁴ and R⁵ are independently selected from the group consisting of a dodecatrienyl moiety, a tetradectrienyl moiety, a hexadecatrienyl moiety, an octadecatrienyl moiety, an icosatrienyl moiety, and a phytanyl moiety, as well as acyl derivatives thereof (e.g., linolenoyl, phytanoyl, etc.). In certain instances, the octadecatrienyl moiety is a linolenyl moiety or a γ-linolenyl moiety. In preferred embodiments, R⁴ and R⁵ are both linolenyl moieties or γ-linolenyl moieties. In particular embodiments, R⁴ and R⁵ independently comprise a backbone of from about 16 to about 22 carbon atoms, and one or both of R⁴ and R⁵ independently comprise at least three, four, five, or six sites of unsaturation.

In some embodiments, the cationic lipid of Formula VI forms a salt (preferably a crystalline salt) with one or more anions. In one particular embodiment, the cationic lipid of Formula VI is the oxalate (e.g., hemioxalate) salt thereof, which is preferably a crystalline salt.

In a particularly preferred embodiment, the cationic lipid of Formula VI has a structure selected from the group consisting of:

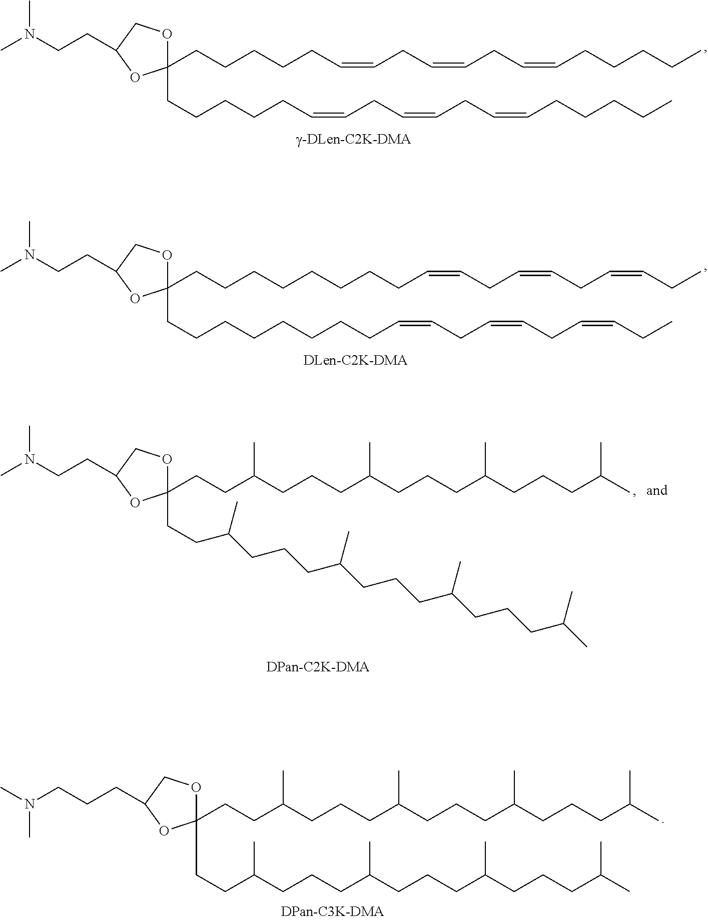

In yet another aspect, cationic lipids of Formula VII having the following structure are useful in the present invention:

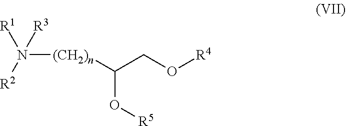

or salts thereof, wherein: R¹ and R² are joined to form an optionally substituted heterocyclic ring of 4 to 6 carbon atoms and 1 or 2 heteroatoms selected from the group consisting of nitrogen (N), oxygen (O), and mixtures thereof; R³ is either absent or is hydrogen (H) or a C₁-C₆ alkyl to provide a quaternary amine; R⁴ and R⁵ are either the same or different and are independently an optionally substituted C₁₂-C₂₄ alkyl, C₁₂-C₂₄ alkenyl, C₁₂-C₂₄ alkynyl, or C₁₂-C₂₄ acyl; and n is 0, 1, 2, 3, or 4.

In some embodiments, R¹ and R² are joined to form a heterocyclic ring of 5 carbon atoms and 1 nitrogen atom. In certain instances, the heterocyclic ring is substituted with a substituent such as a hydroxyl group at the ortho, meta, and/or para positions. In a preferred embodiment, n is 1. In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine. In further embodiments, R⁴ and R⁵ are independently an optionally substituted C₁₂-C₂₀ or C₁₄-C₂₂ alkyl, C₁₂-C₂₀ or C₁₄-C₂₂ alkenyl, C₁₂-C₂₀ or C₁₄-C₂₂ alkynyl, or C₁₂-C₂₀ or C₁₄-C₂₂ acyl.

In certain embodiments, R⁴ and R⁵ are independently selected from the group consisting of a dodecadienyl moiety, a tetradecadienyl moiety, a hexadecadienyl moiety, an octadecadienyl moiety, an icosadienyl moiety, a dodecatrienyl moiety, a tetradectrienyl moiety, a hexadecatrienyl moiety, an octadecatrienyl moiety, an icosatrienyl moiety, and a branched alkyl group as described above (e.g., a phytanyl moiety), as well as acyl derivatives thereof (e.g., linoleoyl, linolenoyl, γ-linolenoyl, phytanoyl, etc.). In some instances, the octadecadienyl moiety is a linoleyl moiety. In other instances, the octadecatrienyl moiety is a linolenyl moiety or a γ-linolenyl moiety. In particular embodiments, R⁴ and R⁵ are both linoleyl moieties, linolenyl moieties, γ-linolenyl moieties, or phytanyl moieties.

In some embodiments, the cationic lipid of Formula VII forms a salt (preferably a crystalline salt) with one or more anions. In one particular embodiment, the cationic lipid of Formula VII is the oxalate (e.g., hemioxalate) salt thereof, which is preferably a crystalline salt.

In a particularly preferred embodiment, the cationic lipid of Formula VII has a structure selected from the group consisting of:

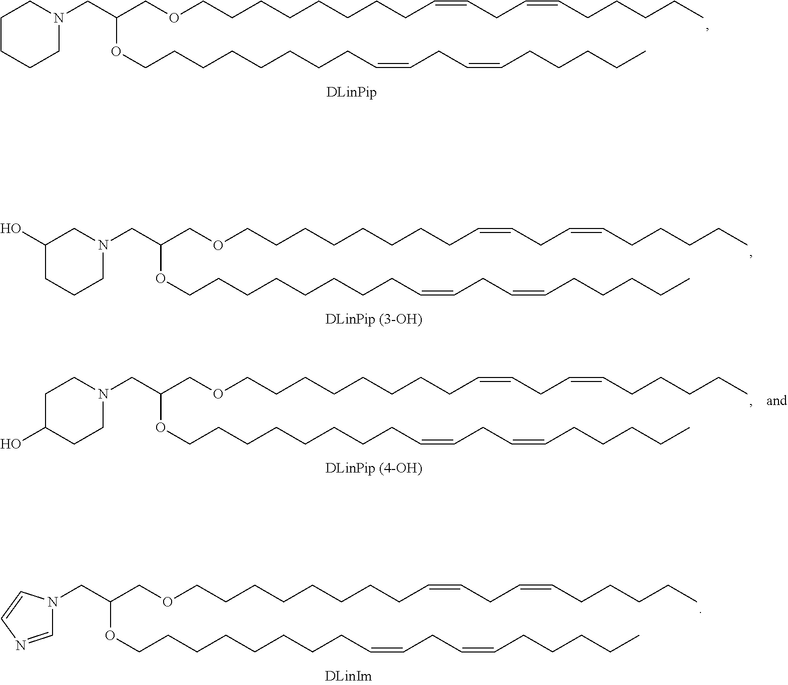 [SMILES: CCCCC/C=C\C/C=C\CCCCCCCCOCC(CN1CCCCC1)OCCCCCCCC/C=C\C/C=C\CCCCC ; CCCCC/C=C\C/C=C\CCCCCCCCOCC(CN1CCCC(O)C1)OCCCCCCCC/C=C\C/C=C\CCCCC ; CCCCC/C=C\C/C=C\CCCCCCCCOCC(CN1CCC(O)CC1)OCCCCCCCC/C=C\C/C=C\CCCCC ; CCCCC/C=C\C/C=C\CCCCCCCCOCC(Cn1ccnc1)OCCCCCCCC/C=C\C/C=C\CCCCC]

In still yet another aspect, cationic lipids of Formula VIII having the following structure are useful in the present invention:

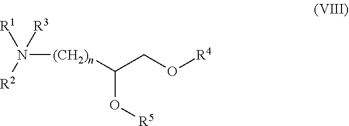

or salts thereof, wherein: R¹ and R² are either the same or different and are independently an optionally substituted C₁-C₆ alkyl, C₂-C₆ alkenyl, or C₂-C₆ alkynyl, or R¹ and R² may join to form an optionally substituted heterocyclic ring of 4 to 6 carbon atoms and 1 or 2 heteroatoms selected from the group consisting of nitrogen (N), oxygen (O), and mixtures thereof; R³ is either absent or is hydrogen (H) or a C₁-C₆ alkyl to provide a quaternary amine; R⁴ and R⁵ are either the same or different and are independently an optionally substituted C₁₂-C₂₄ alkyl, C₁₂-C₂₄ alkenyl, C₁₂-C₂₄ alkynyl, or C₁₂-C₂₄ acyl; and n is 2, 3, or 4.

In some embodiments, R¹ and R² are independently an optionally substituted C₁-C₄ alkyl, C₂-C₄ alkenyl, or C₂-C₄ alkynyl. In a preferred embodiment, R¹ and R² are both methyl groups. In another preferred embodiment, n is 2. In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine. In further embodiments, R⁴ and R⁵ are independently an optionally substituted C₁₂-C₂₀ or C₁₄-C₂₂ alkyl, C₁₂-C₂₀ or C₁₄-C₂₂ alkenyl, C₁₂-C₂₀ or C₁₄-C₂₂ alkynyl, or C₁₂-C₂₀ or C₁₄-C₂₂ acyl.

In certain embodiments, R⁴ and R⁵ are independently selected from the group consisting of a dodecadienyl moiety, a tetradecadienyl moiety, a hexadecadienyl moiety, an octadecadienyl moiety, an icosadienyl moiety, a dodecatrienyl moiety, a tetradectrienyl moiety, a hexadecatrienyl moiety, an octadecatrienyl moiety, an icosatrienyl moiety, and a branched alkyl group as described above (e.g., a phytanyl moiety), as well as acyl derivatives thereof (e.g., linoleoyl, linolenoyl, γ-linolenoyl, phytanoyl, etc.). In some instances, the octadecadienyl moiety is a linoleyl moiety. In other instances, the octadecatrienyl moiety is a linolenyl moiety or a γ-linolenyl moiety. In particular embodiments, R⁴ and R⁵ are both linoleyl moieties, linolenyl moieties, γ-linolenyl moieties, or phytanyl moieties.

In some embodiments, the cationic lipid of Formula VIII forms a salt (preferably a crystalline salt) with one or more anions. In one particular embodiment, the cationic lipid of Formula VIII is the oxalate (e.g., hemioxalate) salt thereof, which is preferably a crystalline salt.

In a particularly preferred embodiment, the cationic lipid of Formula VIII has a structure selected from the group consisting of:

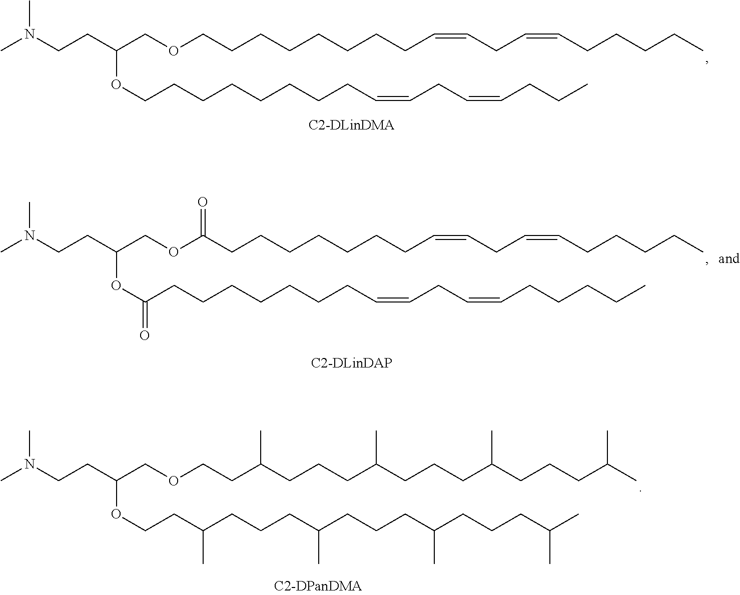

In another aspect, cationic lipids of Formula IX having the following structure are useful in the present invention:

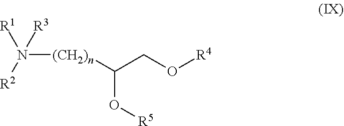

or salts thereof, wherein: R¹ and R² are either the same or different and are independently an optionally substituted C₁-C₆ alkyl, C₂-C₆ alkenyl, or C₂-C₆ alkynyl, or R¹ and R² may join to form an optionally substituted heterocyclic ring of 4 to 6 carbon atoms and 1 or 2 heteroatoms selected from the group consisting of nitrogen (N), oxygen (O), and mixtures thereof; R³ is either absent or is hydrogen (H) or a C₁-C₆ alkyl to provide a quaternary amine; R⁴ and R⁵ are different and are independently an optionally substituted C₁-C₂₄ alkyl, C₂-C₂₄ alkenyl, C₂-C₂₄ alkynyl, or C₁-C₂₄ acyl; and n is 0, 1, 2, 3, or 4.

In some embodiments, R¹ and R² are independently an optionally substituted C₁-C₄ alkyl, C₂-C₄ alkenyl, or C₂-C₄ alkynyl. In a preferred embodiment, R¹ and R² are both methyl groups. In another preferred embodiment, n is 1. In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine. In further embodiments, R⁴ and R⁵ are different and are independently an optionally substituted C₄-C₂₀ alkyl, C₄-C₂₀ alkenyl, C₄-C₂₀ alkynyl, or C₄-C₂₀ acyl.

In some embodiments, R⁴ is an optionally substituted C₁₂-C₂₄ alkyl, C₁₂-C₂₄ alkenyl, C₁₂-C₂₄ alkynyl, or C₁₂-C₂₄ acyl, and R⁵ is an optionally substituted C₄-C₁₀ alkyl, C₄-C₁₀ alkenyl, C₄-C₁₀ alkynyl, or C₄-C₁₀ acyl. In certain instances, R⁴ is an optionally substituted C₁₂-C₂₀ or C₁₄-C₂₂ alkyl, C₁₂-C₂₀ or C₁₄-C₂₂ alkenyl, C₁₂-C₂₀ or C₁₄-C₂₂ alkynyl, or C₁₂-C₂₀ or C₁₄-C₂₂ acyl, and R⁵ is an optionally substituted C₄-C₈ or C₆ alkyl, C₄-C₈ or C₆ alkenyl, C₄-C₈ or C₆ alkynyl, or C₄-C₈ or C₆ acyl.

In other embodiments, R⁴ is an optionally substituted C₄-C₁₀ alkyl, C₄-C₁₀ alkenyl, C₄-C₁₀ alkynyl, or C₄-C₁₀ acyl, and R⁵ is an optionally substituted C₁₂-C₂₄ alkyl, C₁₂-C₂₄ alkenyl, C₁₂-C₂₄ alkynyl, or C₁₂-C₂₄ acyl. In certain instances, R⁴ is an optionally substituted C₄-C₈ or C₆ alkyl, C₄-C₈ or C₆ alkenyl, C₄-C₈ or C₆ alkynyl, or C₄-C₈ or C₆ acyl, and R⁵ is an optionally substituted C₁₂-C₂₀ or C₁₄-C₂₂ alkyl, C₁₂-C₂₀ or C₁₄-C₂₂ alkenyl, C₁₂-C₂₀ or C₁₄-C₂₂ alkynyl, or C₁₂-C₂₀ or C₁₄-C₂₂ acyl.

In particular embodiments, R⁴ is a linoleyl moiety, and R⁵ is a C₆ alkyl moiety, a C₆ alkenyl moiety, an octadecyl moiety, an oleyl moiety, a linolenyl moiety, a γ-linolenyl moiety, or a phytanyl moiety. In other embodiments, one of R⁴ or R⁵ is a phytanyl moiety.

In some embodiments, the cationic lipid of Formula IX forms a salt (preferably a crystalline salt) with one or more anions. In one particular embodiment, the cationic lipid of Formula IX is the oxalate (e.g., hemioxalate) salt thereof, which is preferably a crystalline salt.

In a particularly preferred embodiment, the cationic lipid of Formula IX is an asymmetric lipid having a structure selected from the group consisting of:

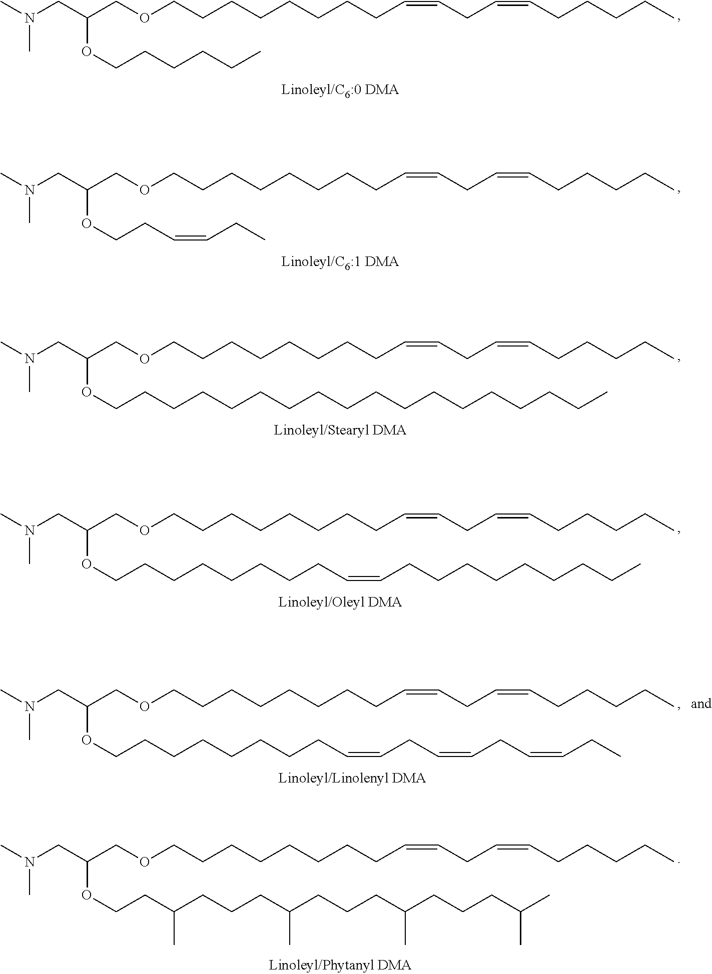

In yet another aspect, cationic lipids of Formula X having the following structure are useful in the present invention:

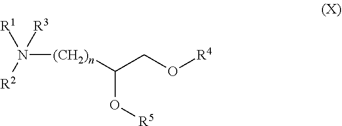

or salts thereof, wherein: R¹ and R² are either the same or different and are independently an optionally substituted C₁-C₆ alkyl, C₂-C₆ alkenyl, or C₂-C₆ alkynyl, or R¹ and R² may join to form an optionally substituted heterocyclic ring of 4 to 6 carbon atoms and 1 or 2 heteroatoms selected from the group consisting of nitrogen (N), oxygen (O), and mixtures thereof; R³ is either absent or is hydrogen (H) or a C₁-C₆ alkyl to provide a quaternary amine; R⁴ and R⁵ are either the same or different and are independently an optionally substituted C₁₂-C₂₄ alkyl, C₁₂-C₂₄ alkenyl, C₁₂-C₂₄ alkynyl, or C₁₂-C₂₄ acyl, wherein at least one of R⁴ and R⁵ comprises at least four sites of unsaturation or a substituted C₁₂-C₂₄ alkyl; and n is 0, 1, 2, 3, or 4.

In some embodiments, R¹ and R² are independently an optionally substituted C₁-C₄ alkyl, C₂-C₄ alkenyl, or C₂-C₄ alkynyl. In a preferred embodiment, R¹ and R² are both methyl groups. In another preferred embodiment, n is 1. In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine. In further embodiments, R⁴ and R⁵ are independently an optionally substituted C₁₂-C₂₀ or C₁₄-C₂₂ alkyl, C₁₂-C₂₀ or C₁₄-C₂₂ alkenyl, C₁₂-C₂₀ or C₁₄-C₂₂ alkynyl, or C₁₂-C₂₀ or C₁₄-C₂₂ acyl.

In embodiments where at least one of R⁴ and R⁵ comprises a branched alkyl group (e.g., a substituted C₁₂-C₂₄ alkyl group), the branched alkyl group may comprise a C₁₂-C₂₄ alkyl having at least 1-6 (e.g., 1, 2, 3, 4, 5, 6, or more) C₁-C₆ alkyl substituents. In particular embodiments, the branched alkyl group comprises a C₁₂-C₂₀ or C₁₄-C₂₂ alkyl with 1-6 (e.g., 1, 2, 3, 4, 5, 6) C₁-C₄ alkyl (e.g., methyl, ethyl, propyl, or butyl) substituents. Preferably, the branched alkyl group comprises a phytanyl (3,7,11,15-tetramethyl-hexadecanyl) moiety.

In alternative embodiments, at least one of R⁴ and R⁵ comprises a branched acyl group (e.g., a substituted C₁₂-C₂₄ acyl group). In certain instances, the branched acyl group may comprise a C₁₂-C₂₄ acyl having at least 1-6 (e.g., 1, 2, 3, 4, 5, 6, or more) C₁-C₆ alkyl substituents. In particular embodiments, the branched acyl group comprises a C₁₂-C₂₀ or C₁₄-C₂₂ acyl with 1-6 (e.g., 1, 2, 3, 4, 5, 6) C₁-C₁ alkyl (e.g., methyl, ethyl, propyl, or butyl) substituents. Preferably, the branched acyl group comprises a phytanoyl (3,7,11,15-tetramethyl-hexadecanoyl) moiety.

In embodiments where at least one of R⁴ and R⁵ comprises at least four sites of unsaturation, the double bonds present in one or both alkyl chains may be in the cis and/or trans configuration. In a particular embodiment, R⁴ and R⁵ independently comprise four, five, or six sites of unsaturation. In some instances, R⁴ comprises four, five, or six sites of unsaturation and R⁵ comprises zero, one, two, three, four, five, or six sites of unsaturation. In other instances, R⁴ comprises zero, one, two, three, four, five, or six sites of unsaturation and R⁵ comprises four, five, or six sites of unsaturation. In a preferred embodiment, both R⁴ and R⁵ comprise four, five, or six sites of unsaturation. In particular embodiments, R⁴ and R⁵ independently comprise a backbone of from about 18 to about 24 carbon atoms, and one or both of R⁴ and R⁵ independently comprise at least four, five, or six sites of unsaturation.

In some embodiments, the cationic lipid of Formula X forms a salt (preferably a crystalline salt) with one or more anions. In one particular embodiment, the cationic lipid of Formula X is the oxalate (e.g., hemioxalate) salt thereof, which is preferably a crystalline salt.

In a particularly preferred embodiment, the cationic lipid of Formula X has a structure selected from the group consisting of:

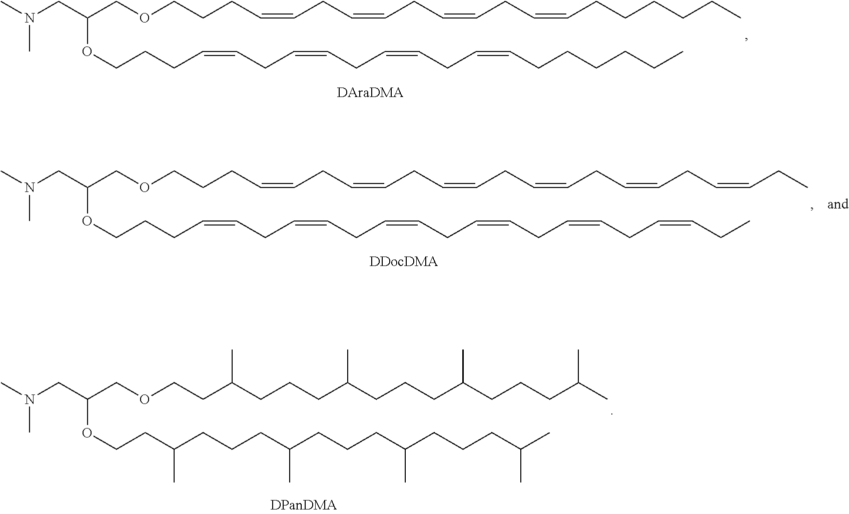

In still yet another aspect, cationic lipids of Formula XI having the following structure are useful in the present invention:

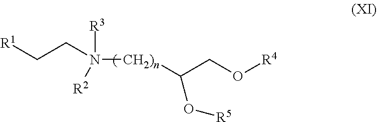

or salts thereof, wherein: R′ is hydrogen (H) or —(CH₂)_{q}—NR⁶R⁷R⁸, wherein: R⁶ and R⁷ are either the same or different and are independently an optionally substituted C₁-C₆ alkyl, C₂-C₆ alkenyl, or C₂-C₆ alkynyl, or R⁶ and R⁷ may join to form an optionally substituted heterocyclic ring of 4 to 6 carbon atoms and 1 or 2 heteroatoms selected from the group consisting of nitrogen (N), oxygen (O), and mixtures thereof; R⁸ is either absent or is hydrogen (H) or a C₁-C₆ alkyl to provide a quaternary amine; and q is 0, 1, 2, 3, or 4; R² is an optionally substituted C₁-C₆ alkyl, C₂-C₆ alkenyl, or C₂-C₆ alkynyl; R³ is either absent or is hydrogen (H) or a C₁-C₆ alkyl to provide a quaternary amine; R⁴ and R⁵ are either the same or different and are independently an optionally substituted C₁₂-C₂₄ alkyl, C₁₂-C₂₄ alkenyl, C₁₂-C₂₄ alkynyl, or C₁₂-C₂₄ acyl; and n is 0, 1, 2, 3, or 4.

In some embodiments, R² is an optionally substituted C₁-C₄ alkyl, C₂-C₄ alkenyl, or C₂-C₄ alkynyl. In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine. In certain embodiments, R⁴ and R⁵ are independently an optionally substituted C₁₂-C₂₀ or C₁₄-C₂₂ alkyl, C₁₂-C₂₀ or C₁₄-C₂₂ alkenyl, C₁₂-C₂₀ or C₁₄-C₂₂ alkynyl, or C₁₂-C₂₀ or C₁₄-C₂₂ acyl.

In further embodiments, R⁶ and R⁷ are independently an optionally substituted C₁-C₄ alkyl, C₂-C₄ alkenyl, or C₂-C₄ alkynyl. In other embodiments, R⁸ is absent when the pH is above the pKₐ of the cationic lipid and R⁸ is hydrogen when the pH is below the pKₐ of the cationic lipid such that the amino head group is protonated. In an alternative embodiment, R⁸ is an optionally substituted C₁-C₄ alkyl to provide a quaternary amine.

In a preferred embodiment, R¹ is hydrogen and R² is an ethyl group. In another preferred embodiment, R⁶ and R⁷ are both methyl groups. In certain instances, n is 1. In certain other instances, q is 1.

In certain embodiments, R⁴ and R⁵ are independently selected from the group consisting of a dodecadienyl moiety, a tetradecadienyl moiety, a hexadecadienyl moiety, an octadecadienyl moiety, an icosadienyl moiety, a dodecatrienyl moiety, a tetradectrienyl moiety, a hexadecatrienyl moiety, an octadecatrienyl moiety, an icosatrienyl moiety, and a branched alkyl group as described above (e.g., a phytanyl moiety), as well as acyl derivatives thereof (e.g., linoleoyl, linolenoyl, γ-linolenoyl, phytanoyl, etc.). In some instances, the octadecadienyl moiety is a linoleyl moiety. In other instances, the octadecatrienyl moiety is a linolenyl moiety or a γ-linolenyl moiety. In particular embodiments, R⁴ and R⁵ are both linoleyl moieties, linolenyl moieties, γ-linolenyl moieties, or phytanyl moieties.

In some embodiments, the cationic lipid of Formula XI forms a salt (preferably a crystalline salt) with one or more anions. In one particular embodiment, the cationic lipid of Formula XI is the oxalate (e.g., hemioxalate) salt thereof, which is preferably a crystalline salt.

In a particularly preferred embodiment, the cationic lipid of Formula XI has a structure selected from the group consisting of:

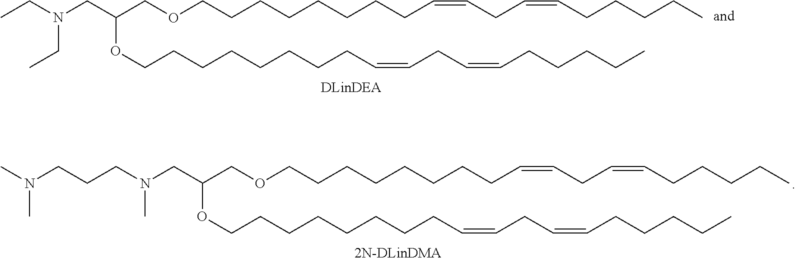

In another aspect, cationic lipids of Formula XII having the following structure are useful in the present invention:

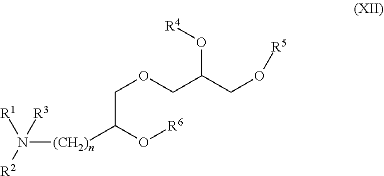

or salts thereof, wherein: R¹ and R² are either the same or different and are independently an optionally substituted C₁-C₆ alkyl, C₂-C₆ alkenyl, or C₂-C₆ alkynyl, or R¹ and R² may join to form an optionally substituted heterocyclic ring of 4 to 6 carbon atoms and 1 or 2 heteroatoms selected from the group consisting of nitrogen (N), oxygen (O), and mixtures thereof; R³ is either absent or is hydrogen (H) or a C₁-C₆ alkyl to provide a quaternary amine; R⁴, R⁵, and R⁶ are either the same or different and are independently an optionally substituted C₁₂-C₂₄ alkyl, C₁₂-C₂₄ alkenyl, C₁₂-C₂₄ alkynyl, or C₁₂-C₂₄ acyl; and n is 0, 1, 2, 3, or 4.

In some embodiments, R¹ and R² are independently an optionally substituted C₁-C₄ alkyl, C₂-C₄ alkenyl, or C₂-C₄ alkynyl. In a preferred embodiment, R¹ and R² are both methyl groups. In another preferred embodiment, n is 1. In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine. In further embodiments, R⁴, R⁵, and R⁶ are independently an optionally substituted C₁₂-C₂₀ or C₁₄-C₂₂ alkyl, C₁₂-C₂₀ or C₁₄-C₂₂ alkenyl, C₁₂-C₂₀ or C₁₄-C₂₂ alkynyl, or C₁₂-C₂₀ or C₁₄-C₂₂ acyl.

In certain embodiments, R⁴, R⁵, and R⁶ are independently selected from the group consisting of a dodecadienyl moiety, a tetradecadienyl moiety, a hexadecadienyl moiety, an octadecadienyl moiety, an icosadienyl moiety, a dodecatrienyl moiety, a tetradectrienyl moiety, a hexadecatrienyl moiety, an octadecatrienyl moiety, an icosatrienyl moiety, and a branched alkyl group as described above (e.g., a phytanyl moiety), as well as acyl derivatives thereof (e.g., linoleoyl, linolenoyl, phytanoyl, etc.). In some instances, the octadecadienyl moiety is a linoleyl moiety. In other instances, the octadecatrienyl moiety is a linolenyl moiety or a γ-linolenyl moiety. In particular embodiments, R⁴, R⁵, and R⁶ are all linoleyl moieties, linolenyl moieties, γ-linolenyl moieties, or phytanyl moieties.

In some embodiments, the cationic lipid of Formula XII forms a salt (preferably a crystalline salt) with one or more anions. In one particular embodiment, the cationic lipid of Formula XII is the oxalate (e.g., hemioxalate) salt thereof, which is preferably a crystalline salt.

In a particularly preferred embodiment, the cationic lipid of Formula XII has a structure selected from the group consisting of:

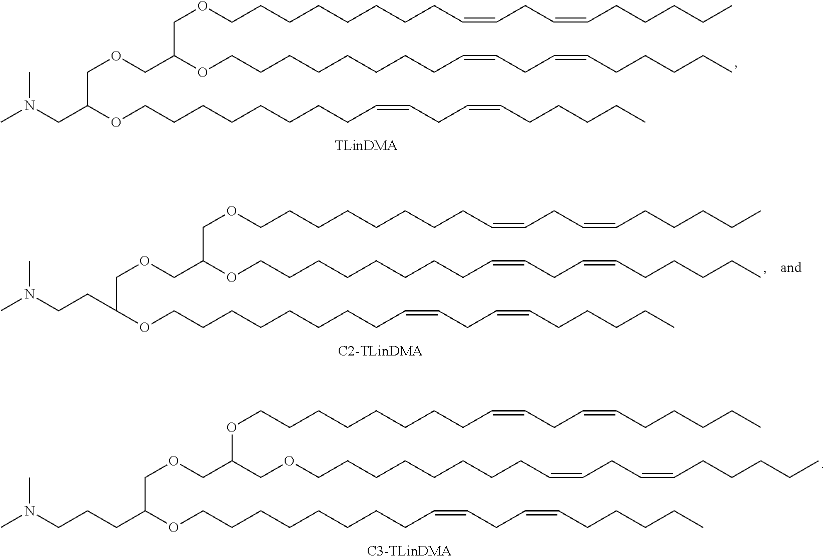

In yet another aspect, cationic lipids of Formula XIII having the following structure are useful in the present invention:

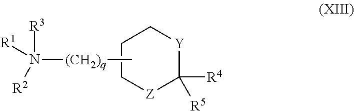

or salts thereof, wherein: R¹ and R² are either the same or different and are independently an optionally substituted C₁-C₆ alkyl, C₂-C₆ alkenyl, or C₂-C₆ alkynyl, or R¹ and R² may join to form an optionally substituted heterocyclic ring of 4 to 6 carbon atoms and 1 or 2 heteroatoms selected from the group consisting of nitrogen (N), oxygen (O), and mixtures thereof; R³ is either absent or is hydrogen (H) or a C₁-C₆ alkyl to provide a quaternary amine; R⁴ and R⁵ are either the same or different and are independently an optionally substituted C₁₂-C₂₄ alkyl, C₁₂-C₂₄ alkenyl, C₁₂-C₂₄ alkynyl, or C₁₂-C₂₄ acyl; q is 0, 1, 2, 3, or 4; and Y and Z are either the same or different and are independently O, S, or NH, wherein if q is 1, R¹ and R² are both methyl groups, R⁴ and R⁵ are both linoleyl moieties, and Y and Z are both 0, then the alkylamino group is attached to one of the two carbons adjacent to Y or Z (i.e., at the ‘4’ or ‘6’ position of the 6-membered ring).

In some embodiments, R¹ and R² are independently an optionally substituted C₁-C₄ alkyl, C₂-C₄ alkenyl, or C₂-C₄ alkynyl. In a preferred embodiment, R¹ and R² are both methyl groups. In another preferred embodiment, q is 2. In a particular embodiments, Y and Z are both oxygen (O). In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine. In further embodiments, R⁴ and R⁵ are independently an optionally substituted C₁₂-C₂₀ or C₁₄-C₂₂ alkyl, C₁₂-C₂₀ or C₁₄-C₂₂ alkenyl, C₁₂-C₂₀ or C₁₄-C₂₂ alkynyl, or C₁₂-C₂₀ or C₁₄-C₂₂ acyl.

In other embodiments, R⁴ and R⁵ are independently selected from the group consisting of a dodecadienyl moiety, a tetradecadienyl moiety, a hexadecadienyl moiety, an octadecadienyl moiety, an icosadienyl moiety, a dodecatrienyl moiety, a tetradectrienyl moiety, a hexadecatrienyl moiety, an octadecatrienyl moiety, an icosatrienyl moiety, and a branched alkyl group as described above (e.g., a phytanyl moiety), as well as acyl derivatives thereof (e.g., linoleoyl, linolenoyl, γ-linolenoyl, phytanoyl, etc.). In some instances, the octadecadienyl moiety is a linoleyl moiety. In other instances, the octadecatrienyl moiety is a linolenyl moiety or a γ-linolenyl moiety. In particular embodiments, R⁴ and R⁵ are both linoleyl moieties, linolenyl moieties, γ-linolenyl moieties, or phytanyl moieties.

The alkylamino head group of Formula XIII may be attached to the ‘4’ or ‘5’ position of the 6-membered ring as shown below in an exemplary embodiment wherein R¹ and R² are both methyl groups:

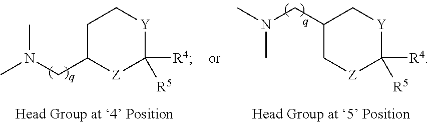

In further embodiments, the 6-membered ring of Formula XIII may be substituted with 1, 2, 3, 4, or 5 independently selected C₁-C₆ alkyl, C₂-C₆ alkenyl, C₂-C₆ alkynyl, C₁-C₆ alkoxyl, or hydroxyl substituents. In one particular embodiment, the 6-membered ring is substituted with 1, 2, 3, 4, or 5 independently selected C₁-C₄ alkyl (e.g., methyl, ethyl, propyl, or butyl) substituents. An exemplary embodiment of a cationic lipid of Formula XIII having a substituted 6-membered ring (methyl group attached to the ‘4’ position) and wherein R¹ and R² are both methyl groups is shown below:

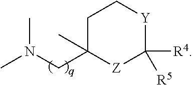

In particular embodiments, the cationic lipids of Formula XIII may be synthesized using 2-hydroxymethyl-1,4-butanediol and 1,3,5-pentanetriol (or 3-methyl-1,3,5-pentanetriol) as starting materials.

In some embodiments, the cationic lipid of Formula XIII forms a salt (preferably a crystalline salt) with one or more anions. In one particular embodiment, the cationic lipid of Formula XIII is the oxalate (e.g., hemioxalate) salt thereof, which is preferably a crystalline salt.

In a particularly preferred embodiment, the cationic lipid of Formula XIII has the structure:

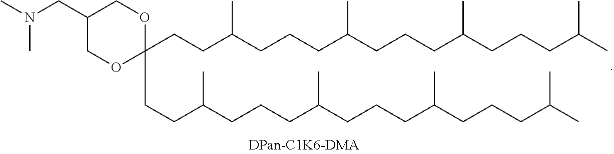 [SMILES: CC(C)CCCC(C)CCCC(C)CCCC(C)CCC1(CCC(C)CCCC(C)CCCC(C)CCCC(C)C)OCC(CN(C)C)CO1]

In still yet another aspect, the present invention provides a cationic lipid of Formula XIV having the following structure:

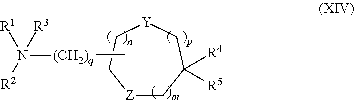

or salts thereof, wherein: R¹ and R² are either the same or different and are independently an optionally substituted C₁-C₆ alkyl, C₂-C₆ alkenyl, or C₂-C₆ alkynyl, or R¹ and R² may join to form an optionally substituted heterocyclic ring of 4 to 6 carbon atoms and 1 or 2 heteroatoms selected from the group consisting of nitrogen (N), oxygen (O), and mixtures thereof; R³ is either absent or is hydrogen (H) or a C₁-C₆ alkyl to provide a quaternary amine; R⁴ and R⁵ are either the same or different and are independently an optionally substituted C₁₂-C₂₄ alkyl, C₁₂-C₂₄ alkenyl, C₁₂-C₂₄ alkynyl, or C₁₂-C₂₄ acyl, wherein at least one of R⁴ and R⁵ comprises at least one site of unsaturation in the trans (E) configuration; m, n, and p are either the same or different and are independently either 0, 1, or 2, with the proviso that m, n, and p are not simultaneously 0; q is 0, 1, 2, 3, or 4; and Y and Z are either the same or different and are independently O, S, or NH.

In some embodiments, R¹ and R² are independently an optionally substituted C₁-C₄ alkyl, C₂-C₄ alkenyl, or C₂-C₄ alkynyl. In a preferred embodiment, R¹ and R² are both methyl groups. In another preferred embodiment, q is 2. In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine. In further embodiments, R⁴ and R⁵ are independently an optionally substituted C₁₂-C₂₀ or C₁₄-C₂₂ alkyl, C₁₂-C₂₀ or C₁₄-C₂₂ alkenyl, C₁₂-C₂₀ or C₁₄-C₂₂ alkynyl, or C₁₂-C₂₀ or C₁₄-C₂₂ acyl.

In certain embodiments, at least one of R⁴ and R⁵ further comprises one, two, three, four, five, six, or more sites of unsaturation in the cis and/or trans configuration. In some instances, R⁴ and R⁵ are independently selected from any of the substituted or unsubstituted alkyl or acyl groups described herein, wherein at least one or both of R⁴ and R⁵ comprises at least one, two, three, four, five, or six sites of unsaturation in the trans configuration. In one particular embodiment, R⁴ and R⁵ independently comprise a backbone of from about 12 to about 22 carbon atoms (e.g., 12, 13, 14, 15, 16, 17, 18, 19, 20, 21, or 22 carbon atoms), and one or both of R⁴ and R⁵ independently comprise at least one, two, three, four, five, or six sites of unsaturation in the trans configuration. In some preferred embodiments, at least one of R⁴ and R⁵ comprises an (E)-heptadecenyl moiety. In other preferred embodiments, R⁴ and R⁵ are both (E)-8-heptadecenyl moieties.

In some embodiments, the cationic lipid of Formula XIV forms a salt (preferably a crystalline salt) with one or more anions. In one particular embodiment, the cationic lipid of Formula XIV is the oxalate (e.g., hemioxalate) salt thereof, which is preferably a crystalline salt.

In a particularly preferred embodiment, the cationic lipid of Formula XIV has the structure:

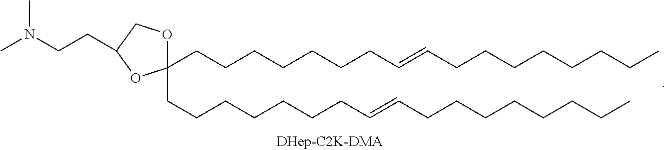 [SMILES: CCCCCCCC/C=C/CCCCCCCC1(CCCCCCC/C=C/CCCCCCCC)OCC(CCN(C)C)O1]

In another aspect, the present invention provides a cationic lipid of Formula XV having the following structure:

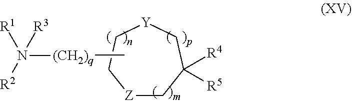

or salts thereof, wherein: R¹ and R² are joined to form an optionally substituted heterocyclic ring of 4 to 6 carbon atoms and 1 or 2 heteroatoms selected from the group consisting of nitrogen (N), oxygen (O), and mixtures thereof; R³ is either absent or is hydrogen (H) or a C₁-C₆ alkyl to provide a quaternary amine; R⁴ and R⁵ are either the same or different and are independently an optionally substituted C₁₂-C₂₄ alkyl, C₁₂-C₂₄ alkenyl, C₁₂-C₂₄ alkynyl, or C₁₂-C₂₄ acyl; m, n, and p are either the same or different and are independently either 0, 1, or 2, with the proviso that m, n, and p are not simultaneously 0; q is 0, 1, 2, 3, or 4; and Y and Z are either the same or different and are independently O, S, or NH.

In some embodiments, R¹ and R² are joined to form a heterocyclic ring of 5 carbon atoms and 1 nitrogen atom. In certain instances, the heterocyclic ring is substituted with a substituent such as a hydroxyl group at the ortho, meta, and/or para positions. In a preferred embodiment, q is 2. In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine. In further embodiments, R⁴ and R⁵ are independently an optionally substituted C₁₂-C₂₀ or C₁₄-C₂₂ alkyl, C₁₂-C₂₀ or C₁₄-C₂₂ alkenyl, C₁₂-C₂₀ or C₁₄-C₂₂ alkynyl, or C₁₂-C₂₀ or C₁₄-C₂₂ acyl.

In certain embodiments, R⁴ and R⁵ are independently selected from the group consisting of a dodecadienyl moiety, a tetradecadienyl moiety, a hexadecadienyl moiety, an octadecadienyl moiety, an icosadienyl moiety, a dodecatrienyl moiety, a tetradectrienyl moiety, a hexadecatrienyl moiety, an octadecatrienyl moiety, an icosatrienyl moiety, and a branched alkyl group as described above (e.g., a phytanyl moiety), as well as acyl derivatives thereof (e.g., linoleoyl, linolenoyl, γ-linolenoyl, phytanoyl, etc.). In some instances, the octadecadienyl moiety is a linoleyl moiety. In other instances, the octadecatrienyl moiety is a linolenyl moiety or a γ-linolenyl moiety. In particular embodiments, R⁴ and R⁵ are both linoleyl moieties, linolenyl moieties, γ-linolenyl moieties, or phytanyl moieties.

In some embodiments, the cationic lipid of Formula XV forms a salt (preferably a crystalline salt) with one or more anions. In one particular embodiment, the cationic lipid of Formula XV is the oxalate (e.g., hemioxalate) salt thereof, which is preferably a crystalline salt.

In a particularly preferred embodiment, the cationic lipid of Formula XV has the structure:

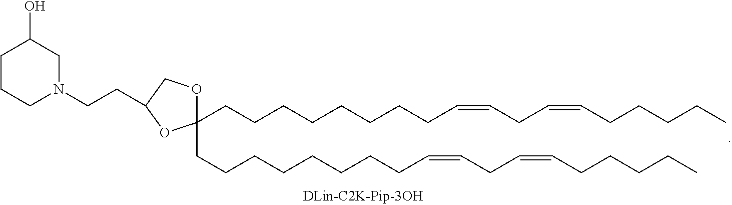 [SMILES: CCCCC/C=C\C/C=C\CCCCCCCCC1(CCCCCCCC/C=C\C/C=C\CCCCC)OCC(CCN2CCCC(O)C2)O1]

In yet another aspect, the present invention provides a cationic lipid of Formula XVI having the following structure:

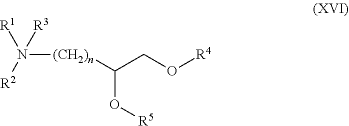

or salts thereof, wherein:
R¹ and R² are either the same or different and are independently an optionally substituted C₁-C₆ alkyl, C₂-C₆ alkenyl, or C₂-C₆ alkynyl, or R¹ and R² may join to form an optionally substituted heterocyclic ring of 4 to 6 carbon atoms and 1 or 2 heteroatoms selected from the group consisting of nitrogen (N), oxygen (O), and mixtures thereof;
R³ is either absent or is hydrogen (H) or a C₁-C₆ alkyl to provide a quaternary amine;
R⁴ and R⁵ are either the same or different and are independently a substituted C₁₂-C₂₄ alkyl; and
n is 0, 1, 2, 3, or 4.

In some embodiments, R¹ and R² are independently an optionally substituted C₁-C₄ alkyl, C₂-C₄ alkenyl, or C₂-C₄ alkynyl. In a preferred embodiment, R¹ and R² are both methyl groups. In one particular embodiment, n is 1. In another particular embodiment, n is 2. In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine.

In embodiments where at least one of R⁴ and R⁵ comprises a branched alkyl group (e.g., a substituted C₁₂-C₂₄ alkyl group), the branched alkyl group may comprise a C₁₂-C₂₄ alkyl having at least 1-6 (e.g., 1, 2, 3, 4, 5, 6, or more) C₁-C₆ alkyl substituents. In particular embodiments, the branched alkyl group comprises a C₁₂-C₂₀ or C₁₄-C₂₂ alkyl with 1-6 (e.g., 1, 2, 3, 4, 5, 6) C₁-C₄ alkyl (e.g., methyl, ethyl, propyl, or butyl) substituents. Preferably, the branched alkyl group comprises a phytanyl (3,7,11,15-tetramethyl-hexadecanyl) moiety. In particular embodiments, R⁴ and R⁵ are both phytanyl moieties.

In alternative embodiments, at least one of R⁴ and R⁵ comprises a branched acyl group (e.g., a substituted C₁₂-C₂₄ acyl group). In certain instances, the branched acyl group may comprise a C₁₂-C₂₄ acyl having at least 1-6 (e.g., 1, 2, 3, 4, 5, 6, or more) C₁-C₆ alkyl substituents. In particular embodiments, the branched acyl group comprises a C₁₂-C₂₀ or C₁₄-C₂₂ acyl with 1-6 (e.g., 1, 2, 3, 4, 5, 6) C₁-C₄ alkyl (e.g., methyl, ethyl, propyl, or butyl) substituents. Preferably, the branched acyl group comprises a phytanoyl (3,7,11,15-tetramethyl-hexadecanoyl) moiety. In particular embodiments, R⁴ and R⁵ are both phytanoyl moieties.

In some embodiments, the cationic lipid of Formula XVI forms a salt (preferably a crystalline salt) with one or more anions. In one particular embodiment, the cationic lipid of Formula XVI is the oxalate (e.g., hemioxalate) salt thereof, which is preferably a crystalline salt.

In a particularly preferred embodiment, the cationic lipid of Formula XVI has a structure selected from the group consisting of:

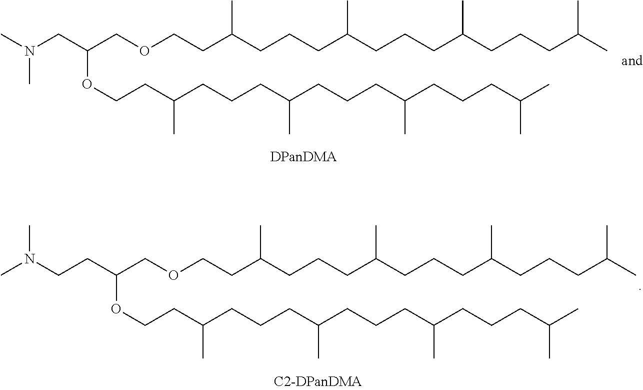

The synthesis of cationic lipids of Formulas V-XVI is described in PCT Application No. PCT/CA2010/001029, filed Jun. 30, 2010, the disclosure of which is herein incorporated by reference in its entirety for all purposes.

In a further aspect, cationic lipids of Formula XVII having the following structure are useful in the present invention:

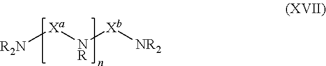

or salts thereof, wherein:
each X^{a} and X^{b}, for each occurrence, is independently a C₁₆ alkylene;
n is 0, 1, 2, 3, 4, or 5;

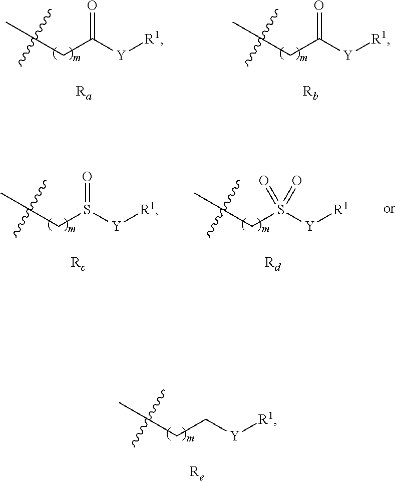

each R is independently H,
   wherein:
at least n+2 of the R moieties in at least about 80% of the molecules of the compound of Formula (XVII) in the preparation are not H;
m is 1, 2, 3 or 4; Y is O, NR², or S;
R¹ is H, alkyl, alkenyl or alkynyl, each of which is optionally substituted with one or more substituents; and
R² is H, alkyl alkenyl or alkynyl, each of which is optionally substituted with one or more substituents;
provided that at least one of R¹ or R² is an alkenyl group comprising at least two sites of unsaturation, and
provided that if n=0, then at least n+3 of the R moieties are not H.

The synthesis of cationic lipids of Formula XVII, as well as additional cationic lipids, is described in U.S. Patent Publication No. 20090023673, the disclosure of which is herein incorporated by reference in its entirety for all purposes.

In another aspect, cationic lipids of Formula XVIII having the following structure are useful in the present invention:

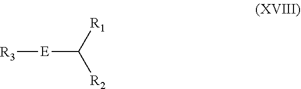

wherein:

R₁ and R₂ are each independently for each occurrence optionally substituted C₁₀-C₃₀ alkyl, optionally substituted C₁₀-C₃₀ alkenyl, optionally substituted C₁₀-C₃₀ alkynyl, optionally substituted C₁₀-C₃₀ acyl, or -linker-ligand; R₃ is H, optionally substituted C₁-C₁₀ alkyl, optionally substituted C₂-C₁₀ alkenyl, optionally substituted C₂-C₁₀ alkynyl, alkylheterocycle, alkylphosphate, alkylphosphorothioate, alkylphosphorodithioate, alkylphosphonates, alkylamines, hydroxyalkyls, ω-aminoalkyls, ω-(substituted)aminoalkyls, ω-phosphoalkyls, ω-thiophosphoalkyls, optionally substituted polyethylene glycol (PEG, mw 100-40K), optionally substituted mPEG (mw 120-40K), heteroaryl, heterocycle, or linker-ligand; E is O, S, N(Q), C(O), C(O)O, OC(O), N(Q)C(O), C(O)N(Q), (Q)N(CO)O, O(CO)N(Q), S(O), NS(O)₂N(Q), S(O)₂, N(Q)S(O)₂, SS, O═N, aryl, heteroaryl, cyclic or heterocycle; and Q is H, alkyl, ω-aminoalkyl, ω-(substituted)aminoalkyl, ω-phosphoalkyl, or ω-thiophosphoalkyl; or a salt or isomer thereof.

In one embodiment, R₁ and R₂ are each independently for each occurrence optionally substituted C₁₀-C₃₀ alkyl, optionally substituted C₁₀-C₃₀ alkoxy, optionally substituted C₁₀-C₃₀ alkenyl, optionally substituted C₁₀-C₃₀ alkenyloxy, optionally substituted C₁₀-C₃₀ alkynyl, optionally substituted C₁₀-C₃₀ alkynyloxy, or optionally substituted C₁₀-C₃₀ acyl.

In another embodiment, R₃ is H, optionally substituted C₁-C₁₀ alkyl, optionally substituted C₂-C₁₀ alkenyl, optionally substituted C₂-C₁₀ alkynyl, optionally substituted alkylheterocycle, optionally substituted heterocycloalkyl, optionally substituted alkylphosphate, optionally substituted phosphoalkyl, optionally substituted alkylphosphorothioate, optionally substituted phosphorothioalkyl, optionally substituted alkylphosphorodithioate, optionally substituted phosphorodithioalkyl, optionally substituted alkylphosphonate, optionally substituted phosphonoalkyl, optionally substituted amino, optionally substituted alkylamino, optionally substituted di(alkyl)amino, optionally substituted aminoalkyl, optionally substituted alkylaminoalkyl, optionally substituted di(alkyl)aminoalkyl, optionally substituted hydroxyalkyl, optionally substituted polyethylene glycol (PEG, mw 100-40K), optionally substituted mPEG (mw 120-40K), optionally substituted heteroaryl, optionally substituted heterocycle, or linker-ligand.

In yet another embodiment, E is —O—, —S—, —N(Q)-, —C(O)—, —C(O)O—, —OC(O)—, —N(Q)C(O)—, —C(O)N(Q)-, —N(Q)C(O)O—, —OC(O)N(Q)-, S(O), —N(Q)S(O)₂N(Q)-, —S(O)₂—, —N(Q)S(O)₂—, —SS—, —O—N═, —C(O)—N(Q)-N═, —N(Q)-N═, —N(Q)-O—, —C(O)S—, arylene, heteroarylene, cyclalkylene, or heterocyclylene; and Q is H, alkyl, ω-aminoalkyl, ω-(substituted)aminoalkyl, ω-phosphoalkyl or ω-thiophosphoalkyl.

In another embodiment, the lipid is a compound of Formula XVIII, wherein E is O, S, N(Q), C(O), C(O)O, OC(O), N(Q)C(O), C(O)N(Q), (Q)N(CO)O, O(CO)N(Q), S(O), NS(O)₂N(Q), S(O)₂, N(Q)S(O)₂, SS, O═N, aryl, heteroaryl, cyclic or heterocycle.

In one embodiment, the lipid is a compound of Formula XVIII, wherein R₃ is H, optionally substituted C₂-C₁₀ alkenyl, optionally substituted C₂-C₁₀ alkynyl, alkylheterocycle, alkylphosphate, alkylphosphorothioate, alkylphosphorodithioate, alkylphosphonates, alkylamines, hydroxyalkyls, ω-aminoalkyls, co-(substituted)aminoalkyls, ω-phosphoalkyls, ω-thiophosphoalkyls, optionally substituted polyethylene glycol (PEG, mw 100-40K), optionally substituted mPEG (mw 120-40K), heteroaryl, heterocycle, or linker-ligand.

In yet another embodiment, the lipid is a compound of Formula XVIII, wherein R₁ and R₂ are each independently for each occurrence optionally substituted C₁₀-C₃₀ alkyl, optionally substituted C₁₀-C₃₀ alkynyl, optionally substituted C₁₀-C₃₀ acyl, or -linker-ligand.

In yet another aspect, cationic lipids of Formula XVIX having the following structure are useful in the present invention:

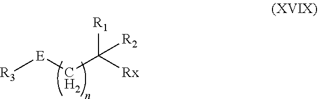

wherein:

E is O, S, N(Q), C(O), C(O)O, OC(O), N(Q)C(O), C(O)N(Q), (Q)N(CO)O, O(CO)N(Q), S(O), NS(O)₂N(Q), S(O)₂, N(Q)S(O)₂, SS, O═N, aryl, heteroaryl, cyclic or heterocycle; Q is H, alkyl, ω-aminoalkyl, ω-(substituted)aminoalkyl, ω-phosphoalkyl, or ω-thiophosphoalkyl; R₁ and R₂ and Rₓ are each independently for each occurrence H, optionally substituted C₁-C₁₀ alkyl, optionally substituted C₁₀-C₃₀ alkyl, optionally substituted C₁₀-C₃₀ alkenyl, optionally substituted C₁₀-C₃₀ alkynyl, optionally substituted C₁₀-C₃₀ acyl, or linker-ligand, provided that at least one of R₁, R₂ and Rₓ is not H; R₃ is H, optionally substituted C₁-C₁₀ alkyl, optionally substituted C₂-C₁₀ alkenyl, optionally substituted C₂-C₁₀ alkynyl, alkylheterocycle, alkylphosphate, alkylphosphorothioate, alkylphosphorodithioate, alkylphosphonates, alkylamines, hydroxyalkyls, ω-aminoalkyls, ω-(substituted)aminoalkyls, ω-phosphoalkyls, ω-thiophosphoalkyls, optionally substituted polyethylene glycol (PEG, mw 100-40K), optionally substituted mPEG (mw 120-40K), heteroaryl, heterocycle, or linker-ligand; and n is 0, 1, 2, or 3; or a salt or isomer thereof.

In some embodiments, each of R₁ and R₂ is independently for each occurrence optionally substituted C₁₀-C₃₀ alkyl, optionally substituted C₁₀-C₃₀ alkenyl, optionally substituted C₁₀-C₃₀ alkynyl, optionally substituted C₁₀-C₃₀ acyl, or linker-ligand. In some embodiments, Rₓ is H or optionally substituted C₁-C₁₀ alkyl. In some embodiments, Rₓ is optionally substituted C₁₀-C₃₀ alkyl, optionally substituted C₁₀-C₃₀ alkenyl, optionally substituted C₁₀-C₃₀ alkynyl, optionally substituted C₁₀-C₃₀ acyl, or linker-ligand.

In one embodiment, R₁ and R₂ are each independently for each occurrence optionally substituted C₁₀-C₃₀ alkyl, optionally substituted C₁₀-C₃₀ alkoxy, optionally substituted C₁₀-C₃₀ alkenyl, optionally substituted C₁₀-C₃₀ alkenyloxy, optionally substituted C₁₀-C₃₀ alkynyl, optionally substituted C₁₀-C₃₀ alkynyloxy, or optionally substituted C₁₀-C₃₀ acyl, or -linker-ligand.

In one embodiment, R₃ is independently for each occurrence H, optionally substituted C₁-C₁₀ alkyl, optionally substituted C₂-C₁₀ alkenyl, optionally substituted C₂-C₁₀ alkynyl, optionally substituted alkylheterocycle, optionally substituted heterocycloalkyl, optionally substituted alkylphosphate, optionally substituted phosphoalkyl, optionally substituted alkylphosphorothioate, optionally substituted phosphorothioalkyl, optionally substituted alkylphosphorodithioate, optionally substituted phosphorodithioalkyl, optionally substituted alkylphosphonate, optionally substituted phosphonoalkyl, optionally substituted amino, optionally substituted alkylamino, optionally substituted di(alkyl) amino, optionally substituted aminoalkyl, optionally substituted alkylaminoalkyl, optionally substituted di(alkyl)aminoalkyl, optionally substituted hydroxyalkyl, optionally substituted polyethylene glycol (PEG, mw 100-40K), optionally substituted mPEG (mw 120-40K), optionally substituted heteroaryl, or optionally substituted heterocycle, or linker-ligand.

Non-limiting examples of cationic lipids of Formula XVIII which may be included in the lipid particles of the present invention include cationic lipids such as (6Z,9Z,28Z,31Z)-heptatriaconta-6,9,28,31-tetraen-19-yl 4-(dimethylamino) butanoate (DLin-M-C3-DMA or “MC3”; also called dilinoleylmethyl 4-(dimethylamino)butanoate) and certain analogs thereof including 3-((6Z,9Z,28Z,31Z)-heptatriaconta-6,9,28,31-tetraen-19-yloxy)-N,N-dimethylpropan-1-amine (MC3 Ether; also called dilinoleylmethyl 4-(dimethylamino)propyl ether), 4-((6Z,9Z,28Z,31Z)-heptatriaconta-6,9,28,31-tetraen-19-yloxy)-N,N-dimethylbutan-1-amine (MC4 Ether; also called dilinoleylmethyl 4-(dimethylamino)butyl ether), and mixtures thereof. The synthesis of cationic lipids of Formula XVIII, as well as additional cationic lipids such as cationic lipids of Formula XVIX, are described in U.S. Provisional Patent Application No. 61/334,104, entitled “Novel Cationic Lipids and Methods of Use Thereof,” filed May 12, 2010, and PCT Publication Nos. WO 2010/054401, WO 2010/054405, WO 2010/054406, and WO 2010/054384, the disclosures of which are herein incorporated by reference in their entirety for all purposes.

In preferred embodiments, the cationic lipid component of the nucleic acid-lipid particles (e.g., SNALP) described herein comprises one or a mixture of two, three, four, or more polyunsaturated cationic lipids of Formulas I-XVIX, wherein each polyunsaturated cationic lipid independently comprises at least one alkyl chain comprising two, three, four, five, or six sites of unsaturation (e.g., double bonds). Examples of preferred polyunsaturated cationic lipids include, but are not limited to, DLinDMA, DLenDMA, γ-DLenDMA, DLin-K-C2-DMA, DLin-K-DMA, DLin-M-C3-DMA, MC3 Ether, MC4 Ether, and a mixture thereof.

In certain instances, other cationic lipids (e.g., saturated, monounsaturated, and/or polyunsaturated cationic lipids) or salts thereof may be included in the lipid particles of the present invention. Such cationic lipids include, but are not limited to, 1,2-dioeylcarbamoyloxy-3-dimethylaminopropane (DO-C-DAP), 1,2-dimyristoleoyl-3-dimethylaminopropane (DMDAP), 1,2-dioleoyl-3-trimethylaminopropane chloride (DOTAP.Cl), dilinoleylmethyl-3-dimethylaminopropionate (DLin-M-K-DMA; also known as DLin-M-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), and mixtures thereof.

The synthesis of cationic lipids such as DO-C-DAP, DMDAP, DOTAP.Cl, DLin-M-K-DMA, as well as additional cationic lipids, is described in PCT Publication No. WO 2010/042877, the disclosure of which is incorporated herein by reference in its entirety for all purposes.

The synthesis of cationic lipids such as DLin-C-DAP, DLinDAC, DLinMA, DLinDAP, DLin-S-DMA, DLin-2-DMAP, DLinTMA.Cl, DLinTAP.Cl, DLinMPZ, DLinAP, DOAP, and DLin-EG-DMA, as well as additional cationic lipids, is described in PCT Publication No. WO 09/086,558, the disclosure of which is herein incorporated by reference in its entirety for all purposes.

The synthesis of cationic lipids such as CLinDMA, as well as additional cationic lipids, is described in U.S. Patent Publication No. 20060240554, the disclosure of which is herein incorporated by reference in its entirety for all purposes.

The synthesis of a number of other cationic lipids and related analogs has been described in U.S. Pat. Nos. 5,208,036; 5,264,618; 5,279,833; 5,283,185; 5,753,613; and 5,785,992; and PCT Publication No. WO 96/10390, the disclosures of which are each herein incorporated by reference in their entirety for all purposes. Additionally, a number of commercial preparations of cationic lipids can be used, such as, e.g., LIPOFECTIN® (including DOTMA and DOPE, available from GIBCO/BRL); LIPOFECTAMINE® (including DOSPA and DOPE, available from GIBCO/BRL); and TRANSFECTAM® (including DOGS, available from Promega Corp.).

In some embodiments, the cationic lipid comprises from about 45 mol % to about 90 mol %, from about 45 mol % to about 85 mol %, from about 45 mol % to about 80 mol %, from about 45 mol % to about 75 mol %, from about 45 mol % to about 70 mol %, from about 45 mol % to about 65 mol %, from about 45 mol % to about 60 mol %, from about 45 mol % to about 55 mol %, from about 50 mol % to about 90 mol %, from about 50 mol % to about 85 mol %, from about 50 mol % to about 80 mol %, from about 50 mol % to about 75 mol %, from about 50 mol % to about 70 mol %, from about 50 mol % to about 65 mol %, from about 50 mol % to about 60 mol %, from about 55 mol % to about 65 mol % or from about 55 mol % to about 70 mol % (or any fraction thereof or range therein) of the total lipid present in the particle.

In certain preferred embodiments, the cationic lipid comprises from about 50 mol % to about 58 mol %, from about 51 mol % to about 59 mol %, from about 51 mol % to about 58 mol %, from about 51 mol % to about 57 mol %, from about 52 mol % to about 58 mol %, from about 52 mol % to about 57 mol %, from about 52 mol % to about 56 mol %, or from about 53 mol % to about 55 mol % (or any fraction thereof or range therein) of the total lipid present in the particle. In particular embodiments, the cationic lipid comprises about 50 mol %, 51 mol %, 52 mol %, 53 mol %, 54 mol %, 55 mol %, 56 mol %, 57 mol %, 58 mol %, 59 mol %, 60 mol %, 61 mol %, 62 mol %, 63 mol %, 64 mol %, or 65 mol % (or any fraction thereof or range therein) of the total lipid present in the particle. In certain other embodiments, the cationic lipid comprises (at least) about 66, 67, 68, 69, 70, 71, 72, 73, 74, 75, 76, 77, 78, 79, 80, 81, 82, 83, 84, 85, 86, 87, 88, 89, or 90 mol % (or any fraction thereof or range therein) of the total lipid present in the particle.

In additional embodiments, the cationic lipid comprises from about 2 mol % to about 60 mol %, from about 5 mol % to about 50 mol %, from about 10 mol % to about 50 mol %, from about 20 mol % to about 50 mol %, from about 20 mol % to about 40 mol %, from about 30 mol % to about 40 mol %, or about 40 mol % (or any fraction thereof or range therein) of the total lipid present in the particle.

Additional percentages and ranges of cationic lipids suitable for use in the lipid particles of the present invention are described in PCT Publication No. WO 09/127,060, U.S. application Ser. No. 12/794,701, filed Jun. 4, 2010, and U.S. application Ser. No. 12/828,189, filed Jun. 30, 2010, the disclosures of which are herein incorporated by reference in their entirety for all purposes.

It should be understood that the percentage of cationic lipid present in the lipid particles of the invention is a target amount, and that the actual amount of cationic lipid present in the formulation may vary, for example, by ±5 mol %. For example, in the 1:57 lipid particle (e.g., SNALP) formulation, the target amount of cationic lipid is 57.1 mol %, but the actual amount of cationic lipid may be ±5 mol %, ±4 mol %, ±3 mol %, ±2 mol %, ±1 mol %, +0.75 mol %, ±0.5 mol %, ±0.25 mol %, or +0.1 mol % of that target amount, with the balance of the formulation being made up of other lipid components (adding up to 100 mol % of total lipids present in the particle). Similarly, in the 7:54 lipid particle (e.g., SNALP) formulation, the target amount of cationic lipid is 54.06 mol %, but the actual amount of cationic lipid may be ±5 mol %, ±4 mol %, ±3 mol %, ±2 mol %, ±1 mol %, ±0.75 mol %, ±0.5 mol %, +0.25 mol %, or ±0.1 mol % of that target amount, with the balance of the formulation being made up of other lipid components (adding up to 100 mol % of total lipids present in the particle).

C. Non-Cationic Lipids

The non-cationic lipids used in the lipid particles of the invention (e.g., SNALP) can be any of a variety of neutral uncharged, zwitterionic, or anionic lipids capable of producing a stable complex.

Non-limiting examples of non-cationic lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

Additional examples of non-cationic lipids include sterols such as cholesterol and derivatives thereof. Non-limiting examples of cholesterol derivatives include polar analogues such as 5α-cholestanol, 5β-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5β-cholestanone, and cholesteryl decanoate; and mixtures thereof. In preferred embodiments, the cholesterol derivative is a polar analogue such as cholesteryl-(4′-hydroxy)-butyl ether. The synthesis of cholesteryl-(2′-hydroxy)-ethyl ether is described in PCT Publication No. WO 09/127,060, the disclosure of which is herein incorporated by reference in its entirety for all purposes.

In some embodiments, the non-cationic lipid present in the lipid particles (e.g., SNALP) comprises or consists of a mixture of one or more phospholipids and cholesterol or a derivative thereof. In other embodiments, the non-cationic lipid present in the lipid particles (e.g., SNALP) comprises or consists of one or more phospholipids, e.g., a cholesterol-free lipid particle formulation. In yet other embodiments, the non-cationic lipid present in the lipid particles (e.g., SNALP) comprises or consists of cholesterol or a derivative thereof, e.g., a phospholipid-free lipid particle formulation.

Other examples of non-cationic lipids suitable for use in the present invention include nonphosphorous containing lipids such as, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyoxylated fatty acid amides, dioctadecyldimethyl ammonium bromide, ceramide, sphingomyelin, and the like.

In some embodiments, the non-cationic lipid comprises from about 10 mol % to about 60 mol %, from about 20 mol % to about 55 mol %, from about 20 mol % to about 45 mol %, from about 20 mol % to about 40 mol %, from about 25 mol % to about 50 mol %, from about 25 mol % to about 45 mol %, from about 30 mol % to about 50 mol %, from about 30 mol % to about 45 mol %, from about 30 mol % to about 40 mol %, from about 35 mol % to about 45 mol %, from about 37 mol % to about 42 mol %, or about 35 mol %, 36 mol %, 37 mol %, 38 mol %, 39 mol %, 40 mol %, 41 mol %, 42 mol %, 43 mol %, 44 mol %, or 45 mol % (or any fraction thereof or range therein) of the total lipid present in the particle.

In embodiments where the lipid particles contain a mixture of phospholipid and cholesterol or a cholesterol derivative, the mixture may comprise up to about 40 mol %, 45 mol %, 50 mol %, 55 mol %, or 60 mol % of the total lipid present in the particle.

In some embodiments, the phospholipid component in the mixture may comprise from about 2 mol % to about 20 mol %, from about 2 mol % to about 15 mol %, from about 2 mol % to about 12 mol %, from about 4 mol % to about 15 mol %, or from about 4 mol % to about 10 mol % (or any fraction thereof or range therein) of the total lipid present in the particle. In certain preferred embodiments, the phospholipid component in the mixture comprises from about 5 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. As a non-limiting example, a 1:57 lipid particle formulation comprising a mixture of phospholipid and cholesterol may comprise a phospholipid such as DPPC or DSPC at about 7 mol % (or any fraction thereof), e.g., in a mixture with cholesterol or a cholesterol derivative at about 34 mol % (or any fraction thereof) of the total lipid present in the particle. As another non-limiting example, a 7:54 lipid particle formulation comprising a mixture of phospholipid and cholesterol may comprise a phospholipid such as DPPC or DSPC at about 7 mol % (or any fraction thereof), e.g., in a mixture with cholesterol or a cholesterol derivative at about 32 mol % (or any fraction thereof) of the total lipid present in the particle.

In other embodiments, the cholesterol component in the mixture may comprise from about 25 mol % to about 45 mol %, from about 25 mol % to about 40 mol %, from about 30 mol % to about 45 mol %, from about 30 mol % to about 40 mol %, from about 27 mol % to about 37 mol %, from about 25 mol % to about 30 mol %, or from about 35 mol % to about 40 mol % (or any fraction thereof or range therein) of the total lipid present in the particle. In certain preferred embodiments, the cholesterol component in the mixture comprises from about 25 mol % to about 35 mol %, from about 27 mol % to about 35 mol %, from about 29 mol % to about 35 mol %, from about 30 mol % to about 35 mol %, from about 30 mol % to about 34 mol %, from about 31 mol % to about 33 mol %, or about 30 mol %, 31 mol %, 32 mol %, 33 mol %, 34 mol %, or 35 mol % (or any fraction thereof or range therein) of the total lipid present in the particle. In other embodiments, the cholesterol component in the mixture comprises about 36, 37, 38, 39, 40, 41, 42, 43, 44, or 45 mol % (or any fraction thereof or range therein) of the total lipid present in the particle. Typically, a 1:57 lipid particle formulation comprising a mixture of phospholipid and cholesterol may comprise cholesterol or a cholesterol derivative at about 34 mol % (or any fraction thereof), e.g., in a mixture with a phospholipid such as DPPC or DSPC at about 7 mol % (or any fraction thereof) of the total lipid present in the particle. Typically, a 7:54 lipid particle formulation comprising a mixture of phospholipid and cholesterol may comprise cholesterol or a cholesterol derivative at about 32 mol % (or any fraction thereof), e.g., in a mixture with a phospholipid such as DPPC or DSPC at about 7 mol % (or any fraction thereof) of the total lipid present in the particle.

In embodiments where the lipid particles are phospholipid-free, the cholesterol or derivative thereof may comprise up to about 25 mol %, 30 mol %, 35 mol %, 40 mol %, 45 mol %, 50 mol %, 55 mol %, or 60 mol % of the total lipid present in the particle.

In some embodiments, the cholesterol or derivative thereof in the phospholipid-free lipid particle formulation may comprise from about 25 mol % to about 45 mol %, from about 25 mol % to about 40 mol %, from about 30 mol % to about 45 mol %, from about 30 mol % to about 40 mol %, from about 31 mol % to about 39 mol %, from about 32 mol % to about 38 mol %, from about 33 mol % to about 37 mol %, from about 35 mol % to about 45 mol %, from about 30 mol % to about 35 mol %, from about 35 mol % to about 40 mol %, or about 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, or 45 mol % (or any fraction thereof or range therein) of the total lipid present in the particle. As a non-limiting example, a 1:62 lipid particle formulation may comprise cholesterol at about 37 mol % (or any fraction thereof) of the total lipid present in the particle. As another non-limiting example, a 7:58 lipid particle formulation may comprise cholesterol at about 35 mol % (or any fraction thereof) of the total lipid present in the particle.

In other embodiments, the non-cationic lipid comprises from about 5 mol % to about 90 mol %, from about 10 mol % to about 85 mol %, from about 20 mol % to about 80 mol %, about 10 mol % (e.g., phospholipid only), or about 60 mol % (e.g., phospholipid and cholesterol or derivative thereof) (or any fraction thereof or range therein) of the total lipid present in the particle.

Additional percentages and ranges of non-cationic lipids suitable for use in the lipid particles of the present invention are described in PCT Publication No. WO 09/127,060, U.S. application Ser. No. 12/794,701, filed Jun. 4, 2010, and U.S. application Ser. No. 12/828,189, filed Jun. 30, 2010, the disclosures of which are herein incorporated by reference in their entirety for all purposes.

It should be understood that the percentage of non-cationic lipid present in the lipid particles of the invention is a target amount, and that the actual amount of non-cationic lipid present in the formulation may vary, for example, by +5 mol %. For example, in the 1:57 lipid particle (e.g., SNALP) formulation, the target amount of phospholipid is 7.1 mol % and the target amount of cholesterol is 34.3 mol %, but the actual amount of phospholipid may be ±2 mol %, ±1.5 mol %, ±1 mol %, ±0.75 mol %, +0.5 mol %, ±0.25 mol %, or ±0.1 mol % of that target amount, and the actual amount of cholesterol may be ±3 mol %, +2 mol %, ±1 mol %, ±0.75 mol %, ±0.5 mol %, ±0.25 mol %, or ±0.1 mol % of that target amount, with the balance of the formulation being made up of other lipid components (adding up to 100 mol % of total lipids present in the particle). Similarly, in the 7:54 lipid particle (e.g., SNALP) formulation, the target amount of phospholipid is 6.75 mol % and the target amount of cholesterol is 32.43 mol %, but the actual amount of phospholipid may be ±2 mol %, ±1.5 mol %, ±1 mol %, ±0.75 mol %, ±0.5 mol %, ±0.25 mol %, or ±0.1 mol % of that target amount, and the actual amount of cholesterol may be ±3 mol %, ±2 mol %, ±1 mol %, ±0.75 mol %, ±0.5 mol %, +0.25 mol %, or ±0.1 mol % of that target amount, with the balance of the formulation being made up of other lipid components (adding up to 100 mol % of total lipids present in the particle).

D. Lipid Conjugates

In addition to cationic and non-cationic lipids, the lipid particles of the invention (e.g., SNALP) may further comprise a lipid conjugate. The conjugated lipid is useful in that it prevents the aggregation of particles. Suitable conjugated lipids include, but are not limited to, PEG-lipid conjugates, POZ-lipid conjugates, ATTA-lipid conjugates, cationic-polymer-lipid conjugates (CPLs), and mixtures thereof. In certain embodiments, the lipid particles comprise either a PEG-lipid conjugate or an ATTA-lipid conjugate together with a CPL. The term “ATTA” or “polyamide” includes, without limitation, compounds described in U.S. Pat. Nos. 6,320,017 and 6,586,559, the disclosures of which are herein incorporated by reference in their entirety for all purposes.

In a preferred embodiment, the lipid conjugate is a PEG-lipid. Examples of PEG-lipids include, but are not limited to, PEG coupled to dialkyloxypropyls (PEG-DAA) as described in, e.g., PCT Publication No. WO 05/026372, PEG coupled to diacylglycerol (PEG-DAG) as described in, e.g., U.S. Patent Publication Nos. 20030077829 and 2005008689, PEG coupled to phospholipids such as phosphatidylethanolamine (PEG-PE), PEG conjugated to ceramides as described in, e.g., U.S. Pat. No. 5,885,613, PEG conjugated to cholesterol or a derivative thereof, and mixtures thereof. The disclosures of these patent documents are herein incorporated by reference in their entirety for all purposes.

Additional PEG-lipids suitable for use in the invention include, without limitation, mPEG2000-1,2-di-O-alkyl-sn3-carbomoylglyceride (PEG-C-DOMG). The synthesis of PEG-C-DOMG is described in PCT Publication No. WO 09/086,558, the disclosure of which is herein incorporated by reference in its entirety for all purposes. Yet additional suitable PEG-lipid conjugates include, without limitation, 148′-(1,2-dimyristoyl-3-propanoxy)-carboxamido-3′,6′-dioxaoctanyl]carbamoyl-ω-methyl-poly(ethylene glycol) (2 KPEG-DMG). The synthesis of 2 KPEG-DMG is described in U.S. Pat. No. 7,404,969, the disclosure of which is herein incorporated by reference in its entirety for all purposes.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, but are not limited to, the following: monomethoxypolyethylene glycol (MePEG-OH), monomethoxypolyethylene glycol-succinate (MePEG-S), monomethoxypolyethylene glycol-succinimidyl succinate (MePEG-S—NHS), monomethoxypolyethylene glycol-amine (MePEG-NH₂), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S—NHS, HO-PEG-NH₂, etc.). Other PEGs such as those described in U.S. Pat. Nos. 6,774,180 and 7,053,150 (e.g., mPEG (20 KDa) amine) are also useful for preparing the PEG-lipid conjugates of the present invention. The disclosures of these patents are herein incorporated by reference in their entirety for all purposes. In addition, monomethoxypolyethyleneglycol-acetic acid (MePEG-CH₂COOH) is particularly useful for preparing PEG-lipid conjugates including, e.g., PEG-DAA conjugates.

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In other instances, the PEG moiety has an average molecular weight of from about 550 daltons to about 1000 daltons, from about 250 daltons to about 1000 daltons, from about 400 daltons to about 1000 daltons, from about 600 daltons to about 900 daltons, from about 700 daltons to about 800 daltons, or about 200, 250, 300, 350, 400, 450, 500, 550, 600, 650, 700, 750, 800, 850, 900, 950, or 1000 daltons. In preferred embodiments, the PEG moiety has an average molecular weight of about 2,000 daltons or about 750 daltons.

In certain instances, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In a preferred embodiment, the linker moiety is a non-ester containing linker moiety. As used herein, the term “non-ester containing linker moiety” refers to a linker moiety that does not contain a carboxylic ester bond (—OC(O)—). Suitable non-ester containing linker moieties include, but are not limited to, amido (—C(O)NH—), amino (—NR—), carbonyl (—C(O)—), carbamate (—NHC(O)O—), urea (—NHC(O)NH—), disulphide (—S—S—), ether (—O—), succinyl (—(O)CCH₂CH₂C(O)—), succinimidyl (—NHC(O)CH₂CH₂C(O)NH—), ether, disulphide, as well as combinations thereof (such as a linker containing both a carbamate linker moiety and an amido linker moiety). In a preferred embodiment, a carbamate linker is used to couple the PEG to the lipid.

In other embodiments, an ester containing linker moiety is used to couple the PEG to the lipid. Suitable ester containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available, or can be isolated or synthesized using conventional techniques known to those of skilled in the art. Phosphatidyl-ethanolamines containing saturated or unsaturated fatty acids with carbon chain lengths in the range of C₁₀ to C₂₀ are preferred. Phosphatidylethanolamines with mono- or diunsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoylphosphatidylethanolamine (DOPE), and distearoyl-phosphatidylethanolamine (DSPE).

The term “diacylglycerol” or “DAG” includes a compound having 2 fatty acyl chains, R¹ and R², both of which have independently between 2 and 30 carbons bonded to the 1- and 2-position of glycerol by ester linkages. The acyl groups can be saturated or have varying degrees of unsaturation. Suitable acyl groups include, but are not limited to, lauroyl (C₁₂), myristoyl (C₁₄), palmitoyl (C₁₆), stearoyl (C₁₈), and icosoyl (C₂₀). In preferred embodiments, R¹ and R² are the same, i.e., R¹ and R² are both myristoyl (i.e., dimyristoyl), R¹ and R² are both stearoyl (i.e., distearoyl), etc. Diacylglycerols have the following general formula:

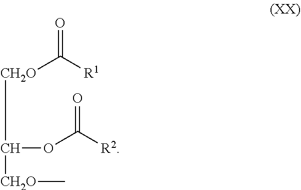

The term “dialkyloxypropyl” or “DAA” includes a compound having 2 alkyl chains, R¹ and R², both of which have independently between 2 and 30 carbons. The alkyl groups can be saturated or have varying degrees of unsaturation. Dialkyloxypropyls have the following general formula:

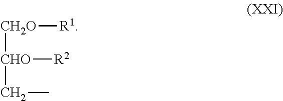

In a preferred embodiment, the PEG-lipid is a PEG-DAA conjugate having the following formula:

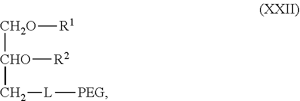

wherein R¹ and R² are independently selected and are long-chain alkyl groups having from about 10 to about 22 carbon atoms; PEG is a polyethyleneglycol; and L is a non-ester containing linker moiety or an ester containing linker moiety as described above. The long-chain alkyl groups can be saturated or unsaturated. Suitable alkyl groups include, but are not limited to, decyl (C₁₀), lauryl (C₁₂), myristyl (C₁₄), palmityl (C₁₆), stearyl (C₁₈), and icosyl (C₂₀). In preferred embodiments, R¹ and R² are the same, i.e., R¹ and R² are both myristyl (i.e., dimyristyl), R¹ and R² are both stearyl (i.e., distearyl), etc.

In Formula XXII above, the PEG has an average molecular weight ranging from about 550 daltons to about 10,000 daltons. In certain instances, the PEG has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In other instances, the PEG moiety has an average molecular weight of from about 550 daltons to about 1000 daltons, from about 250 daltons to about 1000 daltons, from about 400 daltons to about 1000 daltons, from about 600 daltons to about 900 daltons, from about 700 daltons to about 800 daltons, or about 200, 250, 300, 350, 400, 450, 500, 550, 600, 650, 700, 750, 800, 850, 900, 950, or 1000 daltons. In preferred embodiments, the PEG has an average molecular weight of about 2,000 daltons or about 750 daltons. The PEG can be optionally substituted with alkyl, alkoxy, acyl, or aryl groups. In certain embodiments, the terminal hydroxyl group is substituted with a methoxy or methyl group.

In a preferred embodiment, “L” is a non-ester containing linker moiety. Suitable non-ester containing linkers include, but are not limited to, an amido linker moiety, an amino linker moiety, a carbonyl linker moiety, a carbamate linker moiety, a urea linker moiety, an ether linker moiety, a disulphide linker moiety, a succinimidyl linker moiety, and combinations thereof. In a preferred embodiment, the non-ester containing linker moiety is a carbamate linker moiety (i.e., a PEG-C-DAA conjugate). In another preferred embodiment, the non-ester containing linker moiety is an amido linker moiety (i.e., a PEG-A-DAA conjugate). In yet another preferred embodiment, the non-ester containing linker moiety is a succinimidyl linker moiety (i.e., a PEG-S-DAA conjugate).

In particular embodiments, the PEG-lipid conjugate is selected from:

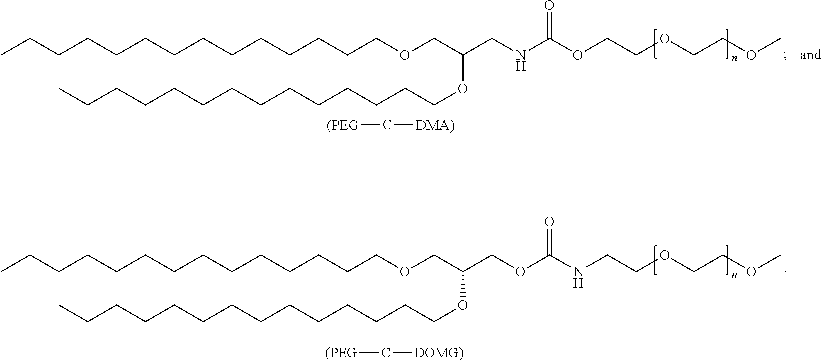

The PEG-DAA conjugates are synthesized using standard techniques and reagents known to those of skill in the art. It will be recognized that the PEG-DAA conjugates will contain various amide, amine, ether, thio, carbamate, and urea linkages. Those of skill in the art will recognize that methods and reagents for forming these bonds are well known and readily available. See, e.g., March, ADVANCED ORGANIC CHEMISTRY (Wiley 1992); Larock, COMPREHENSIVE ORGANIC TRANSFORMATIONS (VCH 1989); and Furniss, VOGEL'S TEXTBOOK OF PRACTICAL ORGANIC CHEMISTRY, 5th ed. (Longman 1989). It will also be appreciated that any functional groups present may require protection and deprotection at different points in the synthesis of the PEG-DAA conjugates. Those of skill in the art will recognize that such techniques are well known. See, e.g., Green and Wuts, PROTECTIVE GROUPS IN ORGANIC SYNTHESIS (Wiley 1991).

Preferably, the PEG-DAA conjugate is a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, or a PEG-distearyloxypropyl (C₁₈) conjugate. In these embodiments, the PEG preferably has an average molecular weight of about 750 or about 2,000 daltons. In one particularly preferred embodiment, the PEG-lipid conjugate comprises PEG2000-C-DMA, wherein the “2000” denotes the average molecular weight of the PEG, the “C” denotes a carbamate linker moiety, and the “DMA” denotes dimyristyloxypropyl. In another particularly preferred embodiment, the PEG-lipid conjugate comprises PEG750-C-DMA, wherein the “750” denotes the average molecular weight of the PEG, the “C” denotes a carbamate linker moiety, and the “DMA” denotes dimyristyloxypropyl. In particular embodiments, the terminal hydroxyl group of the PEG is substituted with a methyl group. Those of skill in the art will readily appreciate that other dialkyloxypropyls can be used in the PEG-DAA conjugates of the present invention.

In addition to the foregoing, it will be readily apparent to those of skill in the art that other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In addition to the foregoing components, the lipid particles (e.g., SNALP) of the present invention can further comprise cationic poly(ethylene glycol) (PEG) lipids or CPLs (see, e.g., Chen et al., Bioconj. Chem., 11:433-437 (2000); U.S. Pat. No. 6,852,334; PCT Publication No. WO 00/62813, the disclosures of which are herein incorporated by reference in their entirety for all purposes).

In some embodiments, the lipid conjugate (e.g., PEG-lipid) comprises from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 0.9 mol % to about 1.6 mol %, from about 0.9 mol % to about 1.8 mol %, from about 1 mol % to about 1.8 mol %, from about 1 mol % to about 1.7 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle.

In other embodiments, the lipid conjugate (e.g., PEG-lipid) comprises from about 0 mol % to about 20 mol %, from about 0.5 mol % to about 20 mol %, from about 2 mol % to about 20 mol %, from about 1.5 mol % to about 18 mol %, from about 2 mol % to about 15 mol %, from about 4 mol % to about 15 mol %, from about 2 mol % to about 12 mol %, from about 5 mol % to about 12 mol %, or about 2 mol % (or any fraction thereof or range therein) of the total lipid present in the particle.

In further embodiments, the lipid conjugate (e.g., PEG-lipid) comprises from about 4 mol % to about 10 mol %, from about 5 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol (or any fraction thereof or range therein) of the total lipid present in the particle.

Additional examples, percentages, and/or ranges of lipid conjugates suitable for use in the lipid particles of the invention are described in PCT Publication No. WO 09/127,060, U.S. application Ser. No. 12/794,701, filed Jun. 4, 2010, U.S. application Ser. No. 12/828,189, filed Jun. 30, 2010, U.S. Provisional Application No. 61/294,828, filed Jan. 13, 2010, U.S. Provisional Application No. 61/295,140, filed Jan. 14, 2010, and PCT Publication No. WO 2010/006282, the disclosures of which are herein incorporated by reference in their entirety for all purposes.

It should be understood that the percentage of lipid conjugate (e.g., PEG-lipid) present in the lipid particles of the invention is a target amount, and that the actual amount of lipid conjugate present in the formulation may vary, for example, by ±2 mol %. For example, in the 1:57 lipid particle (e.g., SNALP) formulation, the target amount of lipid conjugate is 1.4 mol %, but the actual amount of lipid conjugate may be ±0.5 mol %, ±0.4 mol %, ±0.3 mol %, 0.2 mol %, +0.1 mol %, or ±0.05 mol % of that target amount, with the balance of the formulation being made up of other lipid components (adding up to 100 mol % of total lipids present in the particle). Similarly, in the 7:54 lipid particle (e.g., SNALP) formulation, the target amount of lipid conjugate is 6.76 mol %, but the actual amount of lipid conjugate may be 2 mol %, ±1.5 mol %, ±1 mol %, ±0.75 mol %, ±0.5 mol %, ±0.25 mol %, or ±0.1 mol % of that target amount, with the balance of the formulation being made up of other lipid components (adding up to 100 mol % of total lipids present in the particle).

One of ordinary skill in the art will appreciate that the concentration of the lipid conjugate can be varied depending on the lipid conjugate employed and the rate at which the lipid particle is to become fusogenic.

By controlling the composition and concentration of the lipid conjugate, one can control the rate at which the lipid conjugate exchanges out of the lipid particle and, in turn, the rate at which the lipid particle becomes fusogenic. For instance, when a PEG-DAA conjugate is used as the lipid conjugate, the rate at which the lipid particle becomes fusogenic can be varied, for example, by varying the concentration of the lipid conjugate, by varying the molecular weight of the PEG, or by varying the chain length and degree of saturation of the alkyl groups on the PEG-DAA conjugate. In addition, other variables including, for example, pH, temperature, ionic strength, etc. can be used to vary and/or control the rate at which the lipid particle becomes fusogenic. Other methods which can be used to control the rate at which the lipid particle becomes fusogenic will become apparent to those of skill in the art upon reading this disclosure. Also, by controlling the composition and concentration of the lipid conjugate, one can control the lipid particle (e.g., SNALP) size.

### Preparation of Lipid Particles

The lipid particles of the present invention, e.g., SNALP, in which a nucleic acid such as an interfering RNA (e.g., siRNA) is entrapped within the lipid portion of the particle and is protected from degradation, can be formed by any method known in the art including, but not limited to, a continuous mixing method, a direct dilution process, and an in-line dilution process. As described herein, one or more antioxidants such as metal chelators (e.g., EDTA), primary antioxidants, and/or secondary antioxidants may be included at any step or at multiple steps in the process (e.g., prior to, during, and/or after lipid particle formation).

In particular embodiments, the cationic lipids may comprise one, two, or more polyunsaturated cationic lipids such as those set forth in Formulas I-XVIX or salts thereof, alone or in combination with other cationic lipid species. In other embodiments, the non-cationic lipids may comprise one, two, or more lipids including egg sphingomyelin (ESM), distearoylphosphatidylcholine (DSPC), dioleoylphosphatidylcholine (DOPC), 1-palmitoyl-2-oleoyl-phosphatidylcholine (POPC), dipalmitoyl-phosphatidylcholine (DPPC), monomethyl-phosphatidylethanolamine, dimethyl-phosphatidylethanolamine, 14:0 PE (1,2-dimyristoyl-phosphatidylethanolamine (DMPE)), 16:0 PE (1,2-dipalmitoyl-phosphatidylethanolamine (DPPE)), 18:0 PE (1,2-distearoyl-phosphatidylethanolamine (DSPE)), 18:1 PE (1,2-dioleoyl-phosphatidylethanolamine (DOPE)), 18:1 trans PE (1,2-dielaidoyl-phosphatidylethanolamine (DEPE)), 18:0-18:1 PE (1-stearoyl-2-oleoyl-phosphatidylethanolamine (SOPE)), 16:0-18:1 PE (1-palmitoyl-2-oleoyl-phosphatidylethanolamine (POPE)), polyethylene glycol-based polymers (e.g., PEG 2000, PEG 5000, PEG-modified diacylglycerols, or PEG-modified dialkyloxypropyls), cholesterol, derivatives thereof, or combinations thereof.

In certain embodiments, the present invention provides nucleic acid-lipid particles (e.g., SNALP) produced via a continuous mixing method, e.g., a process that includes providing an aqueous solution comprising a nucleic acid (e.g., interfering RNA) in a first reservoir, providing an organic lipid solution in a second reservoir (wherein the lipids present in the organic lipid solution are solubilized in an organic solvent, e.g., a lower alkanol such as ethanol), and mixing the aqueous solution with the organic lipid solution such that the organic lipid solution mixes with the aqueous solution so as to substantially instantaneously produce a lipid vesicle (e.g., liposome) encapsulating the nucleic acid within the lipid vesicle. This process and the apparatus for carrying out this process are described in detail in U.S. Patent Publication No. 20040142025, the disclosure of which is herein incorporated by reference in its entirety for all purposes.

The action of continuously introducing lipid and buffer solutions into a mixing environment, such as in a mixing chamber, causes a continuous dilution of the lipid solution with the buffer solution, thereby producing a lipid vesicle substantially instantaneously upon mixing. As used herein, the phrase “continuously diluting a lipid solution with a buffer solution” (and variations) generally means that the lipid solution is diluted sufficiently rapidly in a hydration process with sufficient force to effectuate vesicle generation. By mixing the aqueous solution comprising a nucleic acid with the organic lipid solution, the organic lipid solution undergoes a continuous stepwise dilution in the presence of the buffer solution (i.e., aqueous solution) to produce a nucleic acid-lipid particle.

The nucleic acid-lipid particles formed using the continuous mixing method typically have a size of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, less than about 120 nm, 110 nm, 100 nm, 90 nm, or 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm (or any fraction thereof or range therein). The particles thus formed do not aggregate and are optionally sized to achieve a uniform particle size.

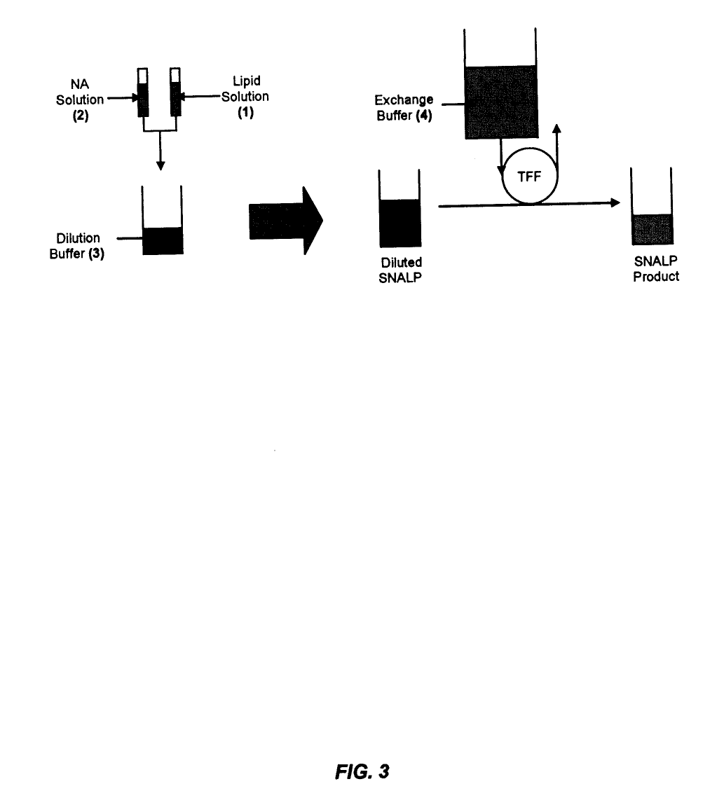

In another embodiment, the present invention provides nucleic acid-lipid particles (e.g., SNALP) produced via a direct dilution process that includes forming a lipid vesicle (e.g., liposome) solution and immediately and directly introducing the lipid vesicle solution into a collection vessel containing a controlled amount of dilution buffer. In preferred aspects, the collection vessel includes one or more elements configured to stir the contents of the collection vessel to facilitate dilution. In one aspect, the amount of dilution buffer present in the collection vessel is substantially equal to the volume of lipid vesicle solution introduced thereto. As a non-limiting example, a lipid vesicle solution in 45% ethanol when introduced into the collection vessel containing an equal volume of dilution buffer will advantageously yield smaller particles. FIG. 3 shows an exemplary direct dilution process for preparing nucleic acid-lipid particles (e.g., SNALP) where one or more antioxidants such as metal chelators (e.g., EDTA), primary antioxidants, and/or secondary antioxidants may be introduced at any step or at multiple steps in the process (see, Example 1).

In yet another embodiment, the present invention provides nucleic acid-lipid particles (e.g., SNALP) produced via an in-line dilution process in which a third reservoir containing dilution buffer is fluidly coupled to a second mixing region. In this embodiment, the lipid vesicle (e.g., liposome) solution formed in a first mixing region is immediately and directly mixed with dilution buffer in the second mixing region. In preferred aspects, the second mixing region includes a T-connector arranged so that the lipid vesicle solution and the dilution buffer flows meet as opposing 180° flows; however, connectors providing shallower angles can be used, e.g., from about 27° to about 180° (e.g., about 90°). A pump mechanism delivers a controllable flow of buffer to the second mixing region. In one aspect, the flow rate of dilution buffer provided to the second mixing region is controlled to be substantially equal to the flow rate of lipid vesicle solution introduced thereto from the first mixing region. This embodiment advantageously allows for more control of the flow of dilution buffer mixing with the lipid vesicle solution in the second mixing region, and therefore also the concentration of lipid vesicle solution in buffer throughout the second mixing process. Such control of the dilution buffer flow rate advantageously allows for small particle size formation at reduced concentrations.

These processes and the apparatuses for carrying out these direct dilution and in-line dilution processes are described in detail in U.S. Patent Publication No. 20070042031, the disclosure of which is herein incorporated by reference in its entirety for all purposes.

The nucleic acid-lipid particles formed using the direct dilution and in-line dilution processes typically have a size of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, less than about 120 nm, 110 nm, 100 nm, 90 nm, or 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm (or any fraction thereof or range therein). The particles thus formed do not aggregate and are optionally sized to achieve a uniform particle size.

If needed, the lipid particles of the invention (e.g., SNALP) can be sized by any of the methods available for sizing liposomes. The sizing may be conducted in order to achieve a desired size range and relatively narrow distribution of particle sizes.

Several techniques are available for sizing the particles to a desired size. One sizing method, used for liposomes and equally applicable to the present particles, is described in U.S. Pat. No. 4,737,323, the disclosure of which is herein incorporated by reference in its entirety for all purposes. Sonicating a particle suspension either by bath or probe sonication produces a progressive size reduction down to particles of less than about 50 nm in size. Homogenization is another method which relies on shearing energy to fragment larger particles into smaller ones. In a typical homogenization procedure, particles are recirculated through a standard emulsion homogenizer until selected particle sizes, typically between about 60 and about 80 nm, are observed. In both methods, the particle size distribution can be monitored by conventional laser-beam particle size discrimination, or QELS.

Extrusion of the particles through a small-pore polycarbonate membrane or an asymmetric ceramic membrane is also an effective method for reducing particle sizes to a relatively well-defined size distribution. Typically, the suspension is cycled through the membrane one or more times until the desired particle size distribution is achieved. The particles may be extruded through successively smaller-pore membranes, to achieve a gradual reduction in size.

In some embodiments, the nucleic acids present in the particles are precondensed as described in, e.g., U.S. patent application Ser. No. 09/744,103, the disclosure of which is herein incorporated by reference in its entirety for all purposes.

In other embodiments, the methods may further comprise adding non-lipid polycations which are useful to effect the lipofection of cells using the present compositions. Examples of suitable non-lipid polycations include, hexadimethrine bromide (sold under the brand name POLYBRENE®, from Aldrich Chemical Co., Milwaukee, Wis., USA) or other salts of hexadimethrine. Other suitable polycations include, for example, salts of poly-L-ornithine, poly-L-arginine, poly-L-lysine, poly-D-lysine, polyallylamine, and polyethyleneimine. Addition of these salts is preferably after the particles have been formed.

In some embodiments, the nucleic acid to lipid ratios (mass/mass ratios) in a formed nucleic acid-lipid particle (e.g., SNALP) will range from about 0.01 to about 0.2, from about 0.05 to about 0.2, from about 0.02 to about 0.1, from about 0.03 to about 0.1, or from about 0.01 to about 0.08. The ratio of the starting materials (input) also falls within this range. In other embodiments, the particle preparation uses about 400 μg nucleic acid per 10 mg total lipid or a nucleic acid to lipid mass ratio of about 0.01 to about 0.08 and, more preferably, about 0.04, which corresponds to 1.25 mg of total lipid per 50 μg of nucleic acid. In other preferred embodiments, the particle has a nucleic acid:lipid mass ratio of about 0.08.

In other embodiments, the lipid to nucleic acid ratios (mass/mass ratios) in a formed nucleic acid-lipid particle (e.g., SNALP) will range from about 1 (1:1) to about 100 (100:1), from about 5 (5:1) to about 100 (100:1), from about 1 (1:1) to about 50 (50:1), from about 2 (2:1) to about 50 (50:1), from about 3 (3:1) to about 50 (50:1), from about 4 (4:1) to about 50 (50:1), from about 5 (5:1) to about 50 (50:1), from about 1 (1:1) to about 25 (25:1), from about 2 (2:1) to about 25 (25:1), from about 3 (3:1) to about 25 (25:1), from about 4 (4:1) to about 25 (25:1), from about 5 (5:1) to about 25 (25:1), from about 5 (5:1) to about 20 (20:1), from about 5 (5:1) to about 15 (15:1), from about 5 (5:1) to about 10 (10:1), or about 5 (5:1), 6 (6:1), 7 (7:1), 8 (8:1), 9 (9:1), 10 (10:1), 11 (11:1), 12 (12:1), 13 (13:1), 14 (14:1), 15 (15:1), 16 (16:1), 17 (17:1), 18 (18:1), 19 (19:1), 20 (20:1), 21 (21:1), 22 (22:1), 23 (23:1), 24 (24:1), or 25 (25:1), or any fraction thereof or range therein. The ratio of the starting materials (input) also falls within this range.

As previously discussed, the conjugated lipid may further include a CPL. A variety of general methods for making SNALP-CPLs (CPL-containing SNALP) are discussed herein. Two general techniques include the “post-insertion” technique, that is, insertion of a CPL into, for example, a pre-formed SNALP, and the “standard” technique, wherein the CPL is included in the lipid mixture during, for example, the SNALP formation steps. The post-insertion technique results in SNALP having CPLs mainly in the external face of the SNALP bilayer membrane, whereas standard techniques provide SNALP having CPLs on both internal and external faces. The method is especially useful for vesicles made from phospholipids (which can contain cholesterol) and also for vesicles containing PEG-lipids (such as PEG-DAAs and PEG-DAGs). Methods of making SNALP-CPLs are taught, for example, in U.S. Pat. Nos. 5,705,385; 6,586,410; 5,981,501; 6,534,484; and 6,852,334; U.S. Patent Publication No. 20020072121; and PCT Publication No. WO 00/62813, the disclosures of which are herein incorporated by reference in their entirety for all purposes.

### Kits

The present invention also provides lipid particles (e.g., SNALP) in kit form. In some embodiments, the kit comprises a container which is compartmentalized for holding the various elements of the lipid particles (e.g., the nucleic acid component and the individual lipid components of the particles). Preferably, the kit comprises a container (e.g., a vial or ampoule) which holds the lipid particles of the invention (e.g., SNALP), wherein the particles are produced by one of the processes set forth herein. In some embodiments, the kit may further comprise one or more antioxidants such as metal chelators (e.g., EDTA), primary antioxidants, and/or secondary antioxidants. In other embodiments, the kit may further comprise an endosomal membrane destabilizer (e.g., calcium ions). The kit typically contains the particle compositions of the present invention, either as a suspension in a pharmaceutically acceptable carrier or in dehydrated form, with instructions for their rehydration (if lyophilized) and administration. In particular embodiments, the particles (whether in a suspension or in dehydrated form) further comprise one or more antioxidants such as metal chelators (e.g., EDTA), primary antioxidants, and/or secondary antioxidants in an amount sufficient to provide particle stability and to prevent or reduce degradation of the particle components.

The lipid particles of the present invention can be tailored to preferentially target particular tissues, organs, or tumors of interest. In certain instances, preferential targeting of lipid particles such as SNALP may be carried out by controlling the composition of the particle itself. In some instances, the 1:57 lipid particle (e.g., SNALP) formulation can be used to preferentially target the liver (e.g., normal liver tissue). In other instances, the 7:54 lipid particle (e.g., SNALP) formulation can be used to preferentially target solid tumors such as liver tumors and tumors outside of the liver. In preferred embodiments, the kits of the invention comprise these liver-directed and/or tumor-directed lipid particles, wherein the particles are present in a container as a suspension or in dehydrated form with one or more antioxidants such as metal chelators (e.g., EDTA), primary antioxidants, and/or secondary antioxidants.

In certain instances, it may be desirable to have a targeting moiety attached to the surface of the lipid particle to further enhance the targeting of the particle. Methods of attaching targeting moieties (e.g., antibodies, proteins, etc.) to lipids (such as those used in the present particles) are known to those of skill in the art.

### Administration of Lipid Particles

Once formed, the lipid particles of the invention (e.g., SNALP) are useful for the introduction of nucleic acids such as interfering RNA into cells. Accordingly, the present invention also provides methods for introducing a nucleic acid such as an interfering RNA (e.g., siRNA) into a cell. In some instances, the cell is a liver cell such as, e.g., a hepatocyte present in liver tissue. In other instances, the cell is a tumor cell such as, e.g., a tumor cell present in a solid tumor. The methods are carried out in vitro or in vivo by first forming the particles as described above and then contacting the particles with the cells for a period of time sufficient for delivery of the nucleic acid to the cells to occur.

The lipid particles of the invention (e.g., SNALP) can be adsorbed to almost any cell type with which they are mixed or contacted. Once adsorbed, the particles can either be endocytosed by a portion of the cells, exchange lipids with cell membranes, or fuse with the cells. Transfer or incorporation of the nucleic acid portion of the particle can take place via any one of these pathways. In particular, when fusion takes place, the particle membrane is integrated into the cell membrane and the contents of the particle combine with the intracellular fluid.

The lipid particles of the invention (e.g., SNALP) can be administered either alone or in a mixture with a pharmaceutically acceptable carrier (e.g., physiological saline or phosphate buffer) selected in accordance with the route of administration and standard pharmaceutical practice. Generally, normal buffered saline (e.g., 135-150 mM NaCl) will be employed as the pharmaceutically acceptable carrier. Other suitable carriers include, e.g., water, buffered water, 0.4% saline, 0.3% glycine, and the like, including glycoproteins for enhanced stability, such as albumin, lipoprotein, globulin, etc. Additional suitable carriers are described in, e.g., REMINGTON'S PHARMACEUTICAL SCIENCES, Mack Publishing Company, Philadelphia, Pa., 17th ed. (1985). As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The phrase “pharmaceutically acceptable” refers to molecular entities and compositions that do not produce an allergic or similar untoward reaction when administered to a human.

The pharmaceutically acceptable carrier is generally added following lipid particle formation. Thus, after the lipid particle (e.g., SNALP) is formed, the particle can be diluted into pharmaceutically acceptable carriers such as normal buffered saline.

The concentration of particles in the pharmaceutical formulations can vary widely, i.e., from less than about 0.05%, usually at or at least about 2 to 5%, to as much as about 10 to 90% by weight, and will be selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, particles composed of irritating lipids may be diluted to low concentrations to lessen inflammation at the site of administration.

The pharmaceutical compositions of the present invention may be sterilized by conventional, well-known sterilization techniques. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, and calcium chloride. Additionally, the particle suspension may include lipid-protective agents which protect lipids against free-radical and lipid-peroxidative damages on storage. Lipophilic free-radical quenchers, such as alphatocopherol, and water-soluble iron-specific chelators, such as ferrioxamine, are suitable.

In some embodiments, the lipid particles of the invention (e.g., SNALP) are particularly useful in methods for the therapeutic delivery of one or more nucleic acids comprising an interfering RNA sequence (e.g., siRNA). In particular, it is an object of this invention to provide in vitro and in vivo methods for treatment of a disease or disorder in a mammal (e.g., a rodent such as a mouse or a primate such as a human, chimpanzee, or monkey) by downregulating or silencing the transcription and/or translation of one or more target nucleic acid sequences or genes of interest. As a non-limiting example, the methods of the invention are useful for in vivo delivery of interfering RNA (e.g., siRNA) to the liver and/or tumor of a mammalian subject. In certain embodiments, the disease or disorder is associated with expression and/or overexpression of a gene and expression or overexpression of the gene is reduced by the interfering RNA (e.g., siRNA). In certain other embodiments, a therapeutically effective amount of the lipid particle may be administered to the mammal. In some instances, an interfering RNA (e.g., siRNA) is formulated into a SNALP, and the particles are administered to patients requiring such treatment. In other instances, cells are removed from a patient, the interfering RNA is delivered in vitro (e.g., using a SNALP described herein), and the cells are reinjected into the patient.

E. In Vivo Administration

Systemic delivery for in vivo therapy, e.g., delivery of a therapeutic nucleic acid to a distal target cell via body systems such as the circulation, has been achieved using nucleic acid-lipid particles such as those described in PCT Publication Nos. WO 05/007196, WO 05/121348, WO 05/120152, and WO 04/002453, the disclosures of which are herein incorporated by reference in their entirety for all purposes.

The nucleic acid-lipid particles of the present invention comprising one or more antioxidants are ideally suited for systemic delivery because they protect the nucleic acid from nuclease degradation in serum, are non-immunogenic, are small in size, and are suitable for repeat dosing. Importantly, the antioxidant or mixture of two, three, or more antioxidants imparts advantageous properties on the nucleic acid-lipid particles by stabilizing both the lipid and nucleic acid components from degradation Particularly preferred antioxidants include EDTA salts such as calcium disodium EDTA (e.g., at least about 20 mM EDTA salt), primary antioxidants such as α-tocopherol or salts thereof (e.g., from about 0.01 mol % to about 10.0 mol %), and/or secondary antioxidants such as ascorbyl palmitate or salts thereof (e.g., from about 0.01 mol % to about 10.0 mol %).

For in vivo administration, administration can be in any manner known in the art, e.g., by injection, oral administration, inhalation (e.g., intransal or intratracheal), transdermal application, or rectal administration. Administration can be accomplished via single or divided doses. The pharmaceutical compositions can be administered parenterally, i.e., intraarticularly, intravenously, intraperitoneally, subcutaneously, or intramuscularly. In some embodiments, the pharmaceutical compositions are administered intravenously or intraperitoneally by a bolus injection (see, e.g., U.S. Pat. No. 5,286,634). Intracellular nucleic acid delivery has also been discussed in Straubringer et al., Methods Enzymol., 101:512 (1983); Mannino et al., Biotechniques, 6:682 (1988); Nicolau et al., Crit. Rev. Ther. Drug Carrier Syst., 6:239 (1989); and Behr, Acc. Chem. Res., 26:274 (1993). Still other methods of administering lipid-based therapeutics are described in, for example, U.S. Pat. Nos. 3,993,754; 4,145,410; 4,235,871; 4,224,179; 4,522,803; and 4,588,578. The lipid particles can be administered by direct injection at the site of disease or by injection at a site distal from the site of disease (see, e.g., Culver, HUMAN GENE THERAPY, MaryAnn Liebert, Inc., Publishers, New York. pp. 70-71 (1994)). The disclosures of the above-described references are herein incorporated by reference in their entirety for all purposes.

In embodiments where the lipid particles of the present invention (e.g., SNALP) are administered intravenously, at least about 5%, 10%, 15%, 20%, or 25% of the total injected dose of the particles is present in plasma about 8, 12, 24, 36, or 48 hours after injection. In other embodiments, more than about 20%, 30%, 40% and as much as about 60%, 70% or 80% of the total injected dose of the lipid particles is present in plasma about 8, 12, 24, 36, or 48 hours after injection. In certain instances, more than about 10% of a plurality of the particles is present in the plasma of a mammal about 1 hour after administration. In certain other instances, the presence of the lipid particles is detectable at least about 1 hour after administration of the particle. In certain embodiments, the presence of a nucleic acid such as an interfering RNA is detectable in cells of the lung, liver, tumor, or at a site of inflammation at about 8, 12, 24, 36, 48, 60, 72 or 96 hours after administration. In other embodiments, downregulation of expression of a target sequence by an interfering RNA (e.g., siRNA) is detectable at about 8, 12, 24, 36, 48, 60, 72 or 96 hours after administration. In yet other embodiments, downregulation of expression of a target sequence by an interfering RNA (e.g., siRNA) occurs preferentially in tumor cells or in cells at a site of inflammation. In further embodiments, the presence or effect of an interfering RNA (e.g., siRNA) in cells at a site proximal or distal to the site of administration or in cells of the lung, liver, or a tumor is detectable at about 12, 24, 48, 72, or 96 hours, or at about 6, 8, 10, 12, 14, 16, 18, 19, 20, 22, 24, 26, or 28 days after administration. In additional embodiments, the lipid particles (e.g., SNALP) of the invention are administered parenterally or intraperitoneally.

The compositions of the present invention, either alone or in combination with other suitable components, can be made into aerosol formulations (i.e., they can be “nebulized”) to be administered via inhalation (e.g., intranasally or intratracheally) (see, Brigham et al., Am. J. Sci., 298:278 (1989)). Aerosol formulations can be placed into pressurized acceptable propellants, such as dichlorodifluoromethane, propane, nitrogen, and the like.

In certain embodiments, the pharmaceutical compositions may be delivered by intranasal sprays, inhalation, and/or other aerosol delivery vehicles. Methods for delivering nucleic acid compositions directly to the lungs via nasal aerosol sprays have been described, e.g., in U.S. Pat. Nos. 5,756,353 and 5,804,212. Likewise, the delivery of drugs using intranasal microparticle resins and lysophosphatidyl-glycerol compounds (U.S. Pat. No. 5,725,871) are also well-known in the pharmaceutical arts. Similarly, transmucosal drug delivery in the form of a polytetrafluoroethylene support matrix is described in U.S. Pat. No. 5,780,045. The disclosures of the above-described patents are herein incorporated by reference in their entirety for all purposes.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions are preferably administered, for example, by intravenous infusion, orally, topically, intraperitoneally, intravesically, or intrathecally.

Generally, when administered intravenously, the lipid particle formulations are formulated with a suitable pharmaceutical carrier. Many pharmaceutically acceptable carriers may be employed in the compositions and methods of the present invention. Suitable formulations for use in the present invention are found, for example, in REMINGTON'S PHARMACEUTICAL SCIENCES, Mack Publishing Company, Philadelphia, Pa., 17th ed. (1985). A variety of aqueous carriers may be used, for example, water, buffered water, 0.4% saline, 0.3% glycine, and the like, and may include glycoproteins for enhanced stability, such as albumin, lipoprotein, globulin, etc. Generally, normal buffered saline (135-150 mM NaCl) will be employed as the pharmaceutically acceptable carrier, but other suitable carriers will suffice. These compositions can be sterilized by conventional liposomal sterilization techniques, such as filtration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. These compositions can be sterilized using the techniques referred to above or, alternatively, they can be produced under sterile conditions. The resulting aqueous solutions may be packaged for use or filtered under aseptic conditions and lyophilized, the lyophilized preparation being combined with a sterile aqueous solution prior to administration.

In certain applications, the lipid particles disclosed herein may be delivered via oral administration to the individual. The particles may be incorporated with excipients and used in the form of ingestible tablets, buccal tablets, troches, capsules, pills, lozenges, elixirs, mouthwash, suspensions, oral sprays, syrups, wafers, and the like (see, e.g., U.S. Pat. Nos. 5,641,515, 5,580,579, and 5,792,451, the disclosures of which are herein incorporated by reference in their entirety for all purposes). These oral dosage forms may also contain the following: binders, gelatin; excipients, lubricants, and/or flavoring agents. When the unit dosage form is a capsule, it may contain, in addition to the materials described above, a liquid carrier. Various other materials may be present as coatings or to otherwise modify the physical form of the dosage unit. Of course, any material used in preparing any unit dosage form should be pharmaceutically pure and substantially non-toxic in the amounts employed.

Typically, these oral formulations may contain at least about 0.1% of the lipid particles or more, although the percentage of the particles may, of course, be varied and may conveniently be between about 1% or 2% and about 60% or 70% or more of the weight or volume of the total formulation. Naturally, the amount of particles in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Formulations suitable for oral administration can consist of: (a) liquid solutions, such as an effective amount of a packaged nucleic acid (e.g., interfering RNA) suspended in diluents such as water, saline, or PEG 400; (b) capsules, sachets, or tablets, each containing a predetermined amount of a nucleic acid (e.g., interfering RNA), as liquids, solids, granules, or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise a nucleic acid (e.g., interfering RNA) in a flavor, e.g., sucrose, as well as pastilles comprising the nucleic acid in an inert base, such as gelatin and glycerin or sucrose and acacia emulsions, gels, and the like containing, in addition to the nucleic acid, carriers known in the art.

In another example of their use, lipid particles can be incorporated into a broad range of topical dosage forms. For instance, a suspension containing nucleic acid-lipid particles such as SNALP can be formulated and administered as gels, oils, emulsions, topical creams, pastes, ointments, lotions, foams, mousses, and the like.

When preparing pharmaceutical preparations of the lipid particles of the invention, it is preferable to use quantities of the particles which have been purified to reduce or eliminate empty particles or particles with nucleic acid associated with the external surface.

The methods of the present invention may be practiced in a variety of hosts. Preferred hosts include mammalian species, such as primates (e.g., humans and chimpanzees as well as other nonhuman primates), canines, felines, equines, bovines, ovines, caprines, rodents (e.g., rats and mice), lagomorphs, and swine.

The amount of particles administered will depend upon the ratio of therapeutic nucleic acid (e.g., interfering RNA) to lipid, the particular therapeutic nucleic acid used, the disease or disorder being treated, the age, weight, and condition of the patient, and the judgment of the clinician, but will generally be between about 0.01 and about 50 mg per kilogram of body weight, preferably between about 0.1 and about 5 mg/kg of body weight, or about 10⁸-10¹⁰ particles per administration (e.g., injection).

F. In Vitro Administration

For in vitro applications, the delivery of nucleic acids (e.g., interfering RNA) can be to any cell grown in culture, whether of plant or animal origin, vertebrate or invertebrate, and of any tissue or type. In preferred embodiments, the cells are animal cells, more preferably mammalian cells, and most preferably human cells (e.g., tumor cells or hepatocytes).

Contact between the cells and the lipid particles, when carried out in vitro, takes place in a biologically compatible medium. The concentration of particles varies widely depending on the particular application, but is generally between about 1 mmol and about 10 mmol. Treatment of the cells with the lipid particles is generally carried out at physiological temperatures (about 37° C.) for periods of time of from about 1 to 48 hours, preferably of from about 2 to 4 hours.

In one group of preferred embodiments, a lipid particle suspension is added to 60-80% confluent plated cells having a cell density of from about 10³ to about 10⁵ cells/ml, more preferably about 2×10⁴ cells/ml. The concentration of the suspension added to the cells is preferably of from about 0.01 to 0.2 μg/ml, more preferably about 0.1 μg/ml.

To the extent that tissue culture of cells may be required, it is well-known in the art. For example, Freshney, Culture of Animal Cells, a Manual of Basic Technique, 3rd Ed., Wiley-Liss, New York (1994), Kuchler et al., Biochemical Methods in Cell Culture and Virology, Dowden, Hutchinson and Ross, Inc. (1977), and the references cited therein provide a general guide to the culture of cells. Cultured cell systems often will be in the form of monolayers of cells, although cell suspensions are also used.

Using an Endosomal Release Parameter (ERP) assay, the delivery efficiency of the SNALP or other lipid particle of the invention can be optimized. An ERP assay is described in detail in U.S. Patent Publication No. 20030077829, the disclosure of which is herein incorporated by reference in its entirety for all purposes. More particularly, the purpose of an ERP assay is to distinguish the effect of various cationic lipids and helper lipid components of SNALP or other lipid particle based on their relative effect on binding/uptake or fusion with/destabilization of the endosomal membrane. This assay allows one to determine quantitatively how each component of the SNALP or other lipid particle affects delivery efficiency, thereby optimizing the SNALP or other lipid particle. Usually, an ERP assay measures expression of a reporter protein (e.g., luciferase, β-galactosidase, green fluorescent protein (GFP), etc.), and in some instances, a SNALP formulation optimized for an expression plasmid will also be appropriate for encapsulating an interfering RNA. In other instances, an ERP assay can be adapted to measure downregulation of transcription or translation of a target sequence in the presence or absence of an interfering RNA (e.g., siRNA). By comparing the ERPs for each of the various SNALP or other lipid particles, one can readily determine the optimized system, e.g., the SNALP or other lipid particle that has the greatest uptake in the cell.

G. Cells for Delivery of Lipid Particles

The compositions and methods of the present invention are used to treat a wide variety of cell types, in vivo and in vitro. Suitable cells include, but are not limited to, hepatocytes, reticuloendothelial cells (e.g., monocytes, macrophages, Kupffer cells, tissue histiocytes, etc.), fibroblast cells, endothelial cells, platelet cells, hematopoietic precursor (stem) cells, keratinocytes, skeletal and smooth muscle cells, osteoblasts, neurons, quiescent lymphocytes, terminally differentiated cells, slow or noncycling primary cells, parenchymal cells, lymphoid cells, epithelial cells, bone cells, and the like.

In particular embodiments, a nucleic acid such as an interfering RNA (e.g., siRNA) is delivered to cancer cells (e.g., cells of a solid tumor) including, but not limited to, liver cancer cells, lung cancer cells, colon cancer cells, rectal cancer cells, anal cancer cells, bile duct cancer cells, small intestine cancer cells, stomach (gastric) cancer cells, esophageal cancer cells, gallbladder cancer cells, pancreatic cancer cells, appendix cancer cells, breast cancer cells, ovarian cancer cells, cervical cancer cells, prostate cancer cells, renal cancer cells, cancer cells of the central nervous system, glioblastoma tumor cells, skin cancer cells, lymphoma cells, choriocarcinoma tumor cells, head and neck cancer cells, osteogenic sarcoma tumor cells, and blood cancer cells.

In vivo delivery of lipid particles such as SNALP encapsulating a nucleic acid (e.g., an interfering RNA) is suited for targeting cells of any cell type. The methods and compositions can be employed with cells of a wide variety of vertebrates, including mammals, such as, e.g., canines, felines, equines, bovines, ovines, caprines, rodents (e.g., mice, rats, and guinea pigs), lagomorphs, swine, and primates (e.g. monkeys, chimpanzees, and humans).

H. Detection of Lipid Particles

In some embodiments, the lipid particles of the present invention (e.g., SNALP) are detectable in the subject at about 1, 2, 3, 4, 5, 6, 7, 8 or more hours. In other embodiments, the lipid particles of the present invention (e.g., SNALP) are detectable in the subject at about 8, 12, 24, 48, 60, 72, or 96 hours, or about 6, 8, 10, 12, 14, 16, 18, 19, 22, 24, 25, or 28 days after administration of the particles. The presence of the particles can be detected in the cells, tissues, or other biological samples from the subject. The particles may be detected, e.g., by direct detection of the particles, detection of a therapeutic nucleic acid such as an interfering RNA (e.g., siRNA) sequence, detection of the target sequence of interest (i.e., by detecting expression or reduced expression of the sequence of interest), or a combination thereof.

1. Detection of Particles

Lipid particles of the invention such as SNALP can be detected using any method known in the art. For example, a label can be coupled directly or indirectly to a component of the lipid particle using methods well-known in the art. A wide variety of labels can be used, with the choice of label depending on sensitivity required, ease of conjugation with the lipid particle component, stability requirements, and available instrumentation and disposal provisions. Suitable labels include, but are not limited to, spectral labels such as fluorescent dyes (e.g., fluorescein and derivatives, such as fluorescein isothiocyanate (FITC) and Oregon Green™; rhodamine and derivatives such Texas red, tetrarhodimine isothiocynate (TRITC), etc., digoxigenin, biotin, phycoerythrin, AMCA, CyDyes™, and the like; radiolabels such as ³H, ¹²⁵I, ³⁵S, ¹⁴C, ³²P, ³³P, etc.; enzymes such as horse radish peroxidase, alkaline phosphatase, etc.; spectral colorimetric labels such as colloidal gold or colored glass or plastic beads such as polystyrene, polypropylene, latex, etc. The label can be detected using any means known in the art.

2. Detection of Nucleic Acids

Nucleic acids (e.g., interfering RNA) are detected and quantified herein by any of a number of means well-known to those of skill in the art. The detection of nucleic acids may proceed by well-known methods such as Southern analysis, Northern analysis, gel electrophoresis, PCR, radiolabeling, scintillation counting, and affinity chromatography. Additional analytic biochemical methods such as spectrophotometry, radiography, electrophoresis, capillary electrophoresis, high performance liquid chromatography (HPLC), thin layer chromatography (TLC), and hyperdiffusion chromatography may also be employed.

The selection of a nucleic acid hybridization format is not critical. A variety of nucleic acid hybridization formats are known to those skilled in the art. For example, common formats include sandwich assays and competition or displacement assays. Hybridization techniques are generally described in, e.g., “Nucleic Acid Hybridization, A Practical Approach,” Eds. Hames and Higgins, IRL Press (1985).

The sensitivity of the hybridization assays may be enhanced through the use of a nucleic acid amplification system which multiplies the target nucleic acid being detected. In vitro amplification techniques suitable for amplifying sequences for use as molecular probes or for generating nucleic acid fragments for subsequent subcloning are known. Examples of techniques sufficient to direct persons of skill through such in vitro amplification methods, including the polymerase chain reaction (PCR), the ligase chain reaction (LCR), Qβ-replicase amplification, and other RNA polymerase mediated techniques (e.g., NASBA™) are found in Sambrook et al., In Molecular Cloning: A Laboratory Manual, Cold Spring Harbor Laboratory Press (2000); and Ausubel et al., SHORT PROTOCOLS IN MOLECULAR BIOLOGY, eds., Current Protocols, Greene Publishing Associates, Inc. and John Wiley & Sons, Inc. (2002); as well as U.S. Pat. No. 4,683,202; PCR Protocols, A Guide to Methods and Applications (Innis et al. eds.) Academic Press Inc. San Diego, Calif. (1990); Arnheim & Levinson (Oct. 1, 1990), C&EN 36; The Journal Of NIH Research, 3:81 (1991); Kwoh et al., Proc. Natl. Acad. Sci. USA, 86:1173 (1989); Guatelli et al., Proc. Natl. Acad. Sci. USA, 87:1874 (1990); Lomell et al., J. Clin. Chem., 35:1826 (1989); Landegren et al., Science, 241:1077 (1988); Van Brunt, Biotechnology, 8:291 (1990); Wu and Wallace, Gene, 4:560 (1989); Barringer et al., Gene, 89:117 (1990); and Sooknanan and Malek, Biotechnology, 13:563 (1995). Improved methods of cloning in vitro amplified nucleic acids are described in U.S. Pat. No. 5,426,039. Other methods described in the art are the nucleic acid sequence based amplification (NASBA™, Cangene, Mississauga, Ontario) and Qβ-replicase systems. These systems can be used to directly identify mutants where the PCR or LCR primers are designed to be extended or ligated only when a select sequence is present. Alternatively, the select sequences can be generally amplified using, for example, nonspecific PCR primers and the amplified target region later probed for a specific sequence indicative of a mutation. The disclosures of the above-described references are herein incorporated by reference in their entirety for all purposes.

Nucleic acids for use as probes, e.g., in in vitro amplification methods, for use as gene probes, or as inhibitor components are typically synthesized chemically according to the solid phase phosphoramidite triester method described by Beaucage et al., Tetrahedron Letts., 22:1859 1862 (1981), e.g., using an automated synthesizer, as described in Needham VanDevanter et al., Nucleic Acids Res., 12:6159 (1984). Purification of polynucleotides, where necessary, is typically performed by either native acrylamide gel electrophoresis or by anion exchange HPLC as described in Pearson et al., J. Chrom., 255:137 149 (1983). The sequence of the synthetic polynucleotides can be verified using the chemical degradation method of Maxam and Gilbert (1980) in Grossman and Moldave (eds.) Academic Press, New York, Methods in Enzymology, 65:499.

An alternative means for determining the level of transcription is in situ hybridization. In situ hybridization assays are well-known and are generally described in Angerer et al., Methods Enzymol., 152:649 (1987). In an in situ hybridization assay, cells are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

### EXAMPLES

The present invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1

### Abrogation of Phosphorothioate to Phosphodiester Conversion in SNALP

### Introduction

Phosphorothioate oligonucleotides are known to be converted to phosphodiesters under certain conditions. This examples illustrates that phosphorothioate modified siRNA encapsulated within SNALP convert to their phosphodiester analogues.

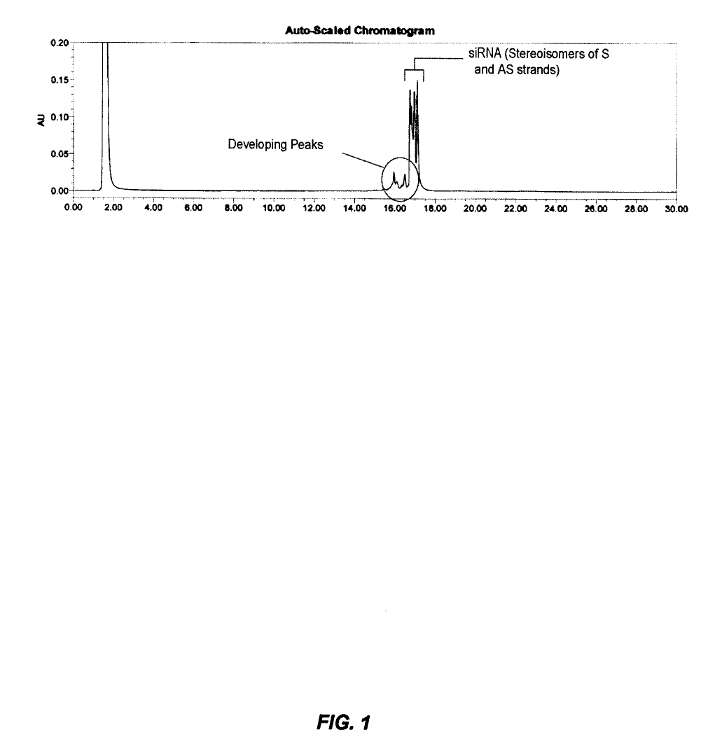

Analysis of SNALP-encapsulated ApoB siRNA containing phosphorothioate linkages by non-denaturing anion exchange HPLC (AX-HPLC) showed new peaks developing upon storage, eluting slightly faster than the ApoB siRNA duplex (FIG. 1).

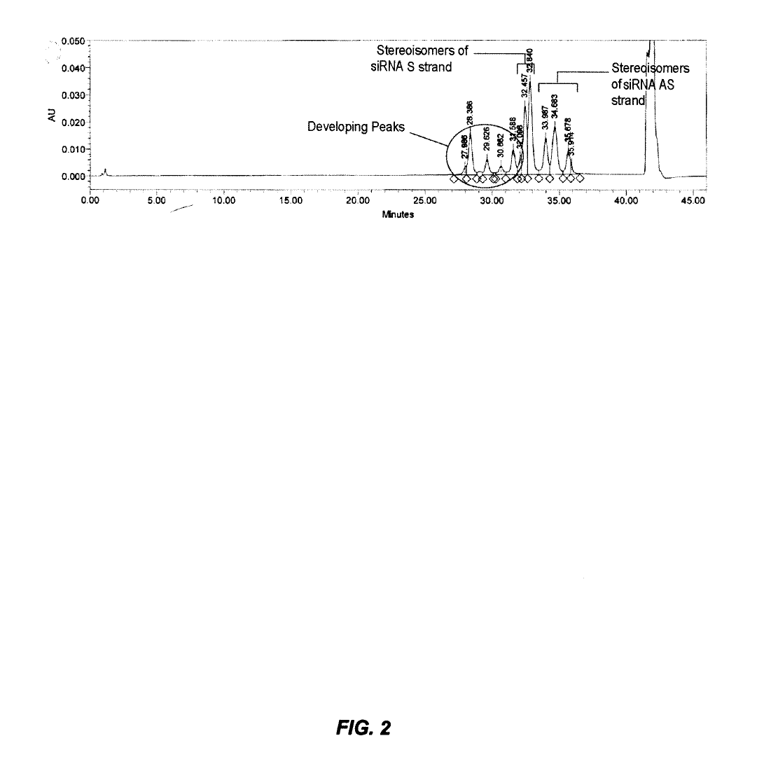

Another HPLC technique, duplex-denaturing Ion Pair Reverse Phase (IPRP), also indicated the development of new products (FIG. 2). The new peaks eluted ahead of the stereoisomer peaks from the sense and antisense strands of the ApoB siRNA duplex, again indicative of conversion to phosphodiesters.

To provide further evidence to support the conversion theory, the various possible phosphodiester degradation products of the ApoB siRNA sense (S) and antisense (AS) strands were synthesized and analyzed by the IPRP-HPLC technique. In this way, all but one of the degradation peaks was identified as co-migrating with the synthesized phosphodiester-containing sequences.

### SNALP Process and Antioxidants

FIG. 3 illustrates a schematic of an exemplary SNALP formulation process and shows at which points antioxidants can be introduced. A Lipid Solution (1) is combined rapidly with a Nucleic Acid (NA) Solution (2) at a T-connector. The resulting mixture is diluted almost instantaneously with a Dilution Buffer (3) to give Diluted SNALP. The buffer mixture of Diluted SNALP is then exchanged with the Exchange Buffer (4) by a process called tangential flow filtration (TFF), so that the final SNALP Product is now suspended in the Exchange Buffer.

Antioxidants can be incorporated into any of the buffers (1-4). Typically, the lipophilic antioxidants were dissolved in the Lipid Solution (1), which is ethanolic. The hydrophilic antioxidants (including EDTA) were added to any or all of the aqueous buffers (2-4).

For antioxidants A-F, the SNALP formulation comprises the lipids DLinDMA (40), DPPC (10), cholesterol (48), and PEG-C-DMA (2) at the molar ratios indicated in parentheses. For antioxidant G, the SNALP formulation comprises the lipids DLinDMA (57.14), DPPC (7.14), cholesterol (34.29), and PEG-C-DMA (1.43) at the molar ratios indicated in parentheses. The ApoB siRNA sequence was used at a 1:6 ratio (wt/wt) to the total lipids.

### Antioxidant Study 1

Antioxidants can be classified in terms of the mechanisms in which they act. Primary antioxidants quench free radicals which are often the source of oxidative pathways. Secondary antioxidants function by decomposing the peroxides that are reactive intermediates of the pathways. Metal chelators function by sequestering the trace metals that promote free radical development.

A panel of antioxidants was selected for evaluation in SNALP formulations. The panel included primary and secondary antioxidants and metal chelators. The panel was organized into groups of both hydrophilic (A-C) and lipophilic (D-F) antioxidants. The lipophilic antioxidants (including α-tocopherol, generally considered to be the most potent antioxidant of the 8 Vitamin E isomers) were incorporated in the ethanolic lipid stock solution, whereas the hydrophilic antioxidants were be added to one or more of several different buffers throughout the SNALP formulation process. SNALP formulations were prepared containing each of the antioxidants and stored at both 5° C. and 37° C. for 1 week or 3 weeks.

The effects of antioxidants on phosphorothioate conversion is summarized in Table 2. Of the hydrophilic antioxidants, citrate (A) and cysteine (C) appeared beneficial in inhibiting phosphorothioate conversion at both 5° C. and 37° C., when used at the higher concentration. However, the use of cysteine led to formation of a precipitate in the sample. Both citrate and cysteine were selected for further investigative studies. None of the lipophilic antioxidants (D-F) appeared to have any meaningful effect on the extent of phosphorothioate degradation at either temperature, including α-tocopherol (E).

TABLE 2
Effects of various antioxidants evaluated in this study after 3 weeks at 5° C. or 37° C.

| | | | | Effect After | Effect After | |
|---|---|---|---|---|---|---|
| | | Where Incorporated | | 3 Weeks at | 3 Weeks at | Additional |
| Description | Antioxidant | (see, FIG. 3) | Concentration | 5° C. | 37° C. | Comments |
| Control | Citrate | Nucleic Acid (2) | 20 mM | N/A | N/A | |
| SNALP | | | | | | |
| Antioxidant | Citrate | Nucleic Acid (2), | 20 mM | −− | − | |
| ‘A’ (Low | | Dilution Buffer (3), | | | | |
| Conc.) | | Exchange Buffer (4) | | | | |
| Antioxidant | Citrate | Nucleic Acid (2), | 100 mM | + | + | |
| ‘A’ (High | | Dilution Buffer (3), | | | | |
| Conc.) | | Exchange Buffer (4) | | | | |
| Antioxidant | Ascorbic | Nucleic Acid (2), | 20 mM | −− | − | |
| ‘B’ (Low | Acid | Dilution Buffer (3), | | | | |
| Conc.) | | Exchange Buffer (4) | | | | |
| Antioxidant | Ascorbic | Nucleic Acid (2), | 100 mM | −− | + | |
| ‘B’ (High | Acid | Dilution Buffer (3), | | | | |
| Conc.) | | Exchange Buffer (4) | | | | |
| Antioxidant | Cysteine | Nucleic Acid (2), | 20 mM | ++ | + | Precipitate |
| ‘C’ (Low | | Dilution Buffer (3), | | | | observed |
| Conc.) | | Exchange Buffer (4) | | | | |
| Antioxidant | Cysteine | Nucleic Acid (2), | 100 mM | ++ | ++ | Precipitate |
| ‘C’ (High | | Dilution Buffer (3), | | | | observed |
| Conc.) | | Exchange Buffer (4) | | | | |
| Antioxidant | Ascorbyl | Lipid Solution (1) | 1% Molar | −− | − | |
| ‘D’ (Low | Palmitate | | Ratio | | | |
| Conc.) | | | | | | |
| Antioxidant | Ascorbyl | Lipid Solution (1) | 5% Molar | − | − | |
| ‘D’ (High | Palmitate | | Ratio | | | |
| Conc.) | | | | | | |
| Antioxidant | Tocopherol | Lipid Solution (1) | 1% Molar | − | − | |
| ‘E’ (Low | | | Ratio | | | |
| Conc.) | | | | | | |
| Antioxidant | Tocopherol | Lipid Solution (1) | 5% Molar | − | − | |
| ‘E’ (High | | | Ratio | | | |
| Conc.) | | | | | | |
| Antioxidant | 2-t-Butyl- | Lipid Solution (1) | 1% Molar | − | − | |
| ‘F’ (Low | methylphenol | | Ratio | | | |
| Conc.) | | | | | | |
| Antioxidant | 2-t-Butyl- | Lipid Solution (1) | 5% Molar | − | − | |
| ‘F’ (High | methylphenol | | Ratio | | | |
| Conc.) | | | | | | |

Each antioxidant was formulated at both a high and low concentration within SNALP and assessed after 3 weeks at either 5° C. or 37° C. Key: ++ = No conversion products observed. + = Beneficial antioxidant effect compared to Control SNALP. − = No different to Control SNALP. −− = Negative effect compared to Control SNALP. All formulations contained 20 mM citrate in the nucleic acid solution, except for Antioxidant ‘A’ (High Conc.), which contained 100 mM citrate.

Duplex integrity in the SNALP was examined using IPRP-HPLC after storage for 1 week at either 5° C. or 37° C. Results are tabulated in Table 3. SNALP comprising a higher concentration of citrate (e.g., 100 mM citrate) was particularly effective at reducing siRNA payload degradation at both temperatures.

TABLE 3
Oligo-HPLC data showing duplex purity after 1 week at 5° C. or 37° C.

| | | | | | 1 Week, 5° C. | 1 Week, 37° C. | |
|---|---|---|---|---|---|---|---|
| | | Where Incorporated | | | IPRP-HPLC | IPRP-HPLC | Additional |
| Description | Antioxidant | (see, FIG. 3) | Concentration | | AUC % Purity | AUC % Purity | Comments |
| Control | Citrate | Nucleic Acid (2) | 20 | mM | 96.0 | 75.6 | |
| SNALP | | | | | | | |
| Antioxidant | Citrate | Nucleic Acid (2) | 20 | mM | 93.3 | 80.9 | |
| ‘A’ (Low | | Dilution Buffer (3) | | | | | |
| Conc.) | | Exchange Buffer (4) | | | | | |
| Antioxidant | Citrate | Nucleic Acid (2) | 100 | mM | 96.4 | 94.3 | |
| ‘A’ (High | | Dilution Buffer (3) | | | | | |
| Conc.) | | Exchange Buffer (4) | | | | | |
| Antioxidant | Ascorbic | Nucleic Acid (2) | 20 | mM | 94.9 | 83.7 | |
| ‘B’ (Low | Acid | Dilution Buffer (3) | | | | | |
| Conc.) | | Exchange Buffer (4) | | | | | |
| Antioxidant | Ascorbic | Nucleic Acid (2) | 100 | mM | 92.4 | 88.3 | |
| ‘B’ (High | Acid | Dilution Buffer (3) | | | | | |
| Conc.) | | Exchange Buffer (4) | | | | | |
| Antioxidant | Cysteine | Nucleic Acid (2) | 20 | mM | 97.2 | 90.9 | Precipitate |
| ‘C’ (Low | | Dilution Buffer (3) | | | | | observed |
| Conc.) | | Exchange Buffer (4) | | | | | |
| Antioxidant | Cysteine | Nucleic Acid (2) | 100 | mM | 96.5 | 94.3 | Precipitate |
| ‘C’ (High | | Dilution Buffer (3) | | | | | observed |
| Conc.) | | Exchange Buffer (4) | | | | | |
| Antioxidant | Ascorbyl | Lipid Solution (1) | 1 | mol % | 97.5 | 93.3 | |
| ‘D’ (Low | Palmitate | | | | | | |
| Conc.) | | | | | | | |
| Antioxidant | Ascorbyl | Lipid Solution (1) | 5 | mol % | 97.0 | 83.9 | |
| ‘D’ (High | Palmitate | | | | | | |
| Conc.) | | | | | | | |
| Antioxidant | Tocopherol | Lipid Solution (1) | 1 | mol % | 96.4 | 54.5 | |
| ‘E’ (Low | | | | | | | |
| Conc.) | | | | | | | |
| Antioxidant | Tocopherol | Lipid Solution (1) | 5 | mol % | 90.6 | 38.9 | |
| ‘E’ (High | | | | | | | |
| Conc.) | | | | | | | |
| Antioxidant | 2-t-Butyl- | Lipid Solution (1) | 1 | mol % | 98.7 | 89.2 | |
| ‘F’ (Low | methylphenol | | | | | | |
| Conc.) | | | | | | | |
| Antioxidant | 2-t-Butyl- | Lipid Solution (1) | 5 | mol % | 98.2 | 81.2 | |
| ‘F’ (High | methylphenol | | | | | | |
| Conc.) | | | | | | | |

### Antioxidant Study 2

Citrate (A) and cysteine (C) were re-evaluated with a more rigorous examination of the method of incorporation into SNALP. Because citrate (A) is known to function by means of metal chelation, the sodium salt of EDTA (Na-EDTA), another metal-chelating agent, was included. A more versatile, amphiphilic antioxidant, dihydrolipoic acid (G), which could be used in either hydrophilic or lipophilic environments, was tested in the second panel. The SNALP formulation used in this study comprises the following lipids: PEG2000-C-DMA (1.43 mol %); DLinDMA (57.14 mol %); cholesterol (34.29 mol %); and DPPC (7.14 mol %).

After storage for 3 months at both 5° C. and ambient temperature, duplex integrity in the SNALP was examined using IPRP-HPLC. Results are tabulated in Table 4. At 5° C., whereas nearly 30% of the siRNA payload appeared to have been degraded/converted in the control SNALP, the EDTA-containing SNALP exhibited only very minor losses. SNALP comprising citrate (A) fair better than the control, with 100 mM citrate being particularly effective when added to all of the aqueous buffers (2-4). However, SNALP containing cysteine (C) and dihydrolipoic acid (G) actually seemed to exacerbate the degradation. These trends were reflected in the samples stored at ambient temperature. The results of this second study clearly established EDTA as the most effective of the tested antioxidants. These results further demonstrated that a higher concentration of citrate (e.g., 100 mM citrate) was also particularly effective at reducing payload degradation.

TABLE 4
IPRP-HPLC data showing duplex purity after 3 months
storage at 5° C. or 37° C.

| | | | | 3 Months, | 3 Months, |
|---|---|---|---|---|---|
| | | | | 5° C. | Ambient |
| | | | | IPRP- | IPRP- |
| | | Where | | HPLC | HPLC |
| | | Incorporated | | AUC % | AUC % |
| Description | Antioxidant | (see, FIG. 3) | Concentration | Purity | Purity |
| ApoB siRNA | None | N/A | N/A | 97.6 | 95.4 |
| Std (Stored at | | | | | |
| −20° C.) | | | | | |
| CTRL | Citrate | Nucleic Acid | 20 mM | 70.0 | 52.8 |
| SNALP | | (2) | | | |
| AntiOx ‘A’ | Citrate | Nucleic Acid | 100 mM | 67.1 | 51.1 |
| (Method 1) | | (2) | | | |
| AntiOx ‘A’ | Citrate | Nucleic Acid | 100 mM | 87.7 | 81.2 |
| (Method 2) | | (2), | | | |
| | | Dilution Buffer | | | |
| | | (3), | | | |
| | | Exchange | | | |
| | | Buffer (4) | | | |
| AntiOx ‘A’ | Citrate | Nucleic Acid | 60 mM | 83.7 | 73.8 |
| (Method 3) | | (2), | | | |
| | | Dilution Buffer | | | |
| | | (3), | | | |
| | | Exchange | | | |
| | | Buffer (4) | | | |
| EDTA-1a | Sodium | Nucleic Acid | 20 mM | 93.4 | 88.3 |
| (Low Conc) | EDTA | (2), | | | |
| | | Dilution Buffer | | | |
| | | (3), | | | |
| | | Exchange | | | |
| | | Buffer (4) | | | |
| EDTA-1b | Sodium | Nucleic Acid | 100 mM | 94.1 | 92.6 |
| (High Cone) | EDTA | (2), | | | |
| | | Dilution Buffer | | | |
| | | (3), | | | |
| | | Exchange | | | |
| | | Buffer (4) | | | |
| AntiOx ‘C’ | Cysteine | Nucleic Acid | 100 mM | 66.0 | 50.2 |
| (Method 2) | | (2) | | | |
| AntiOx ‘G’ | Dihydrolipoic | Lipid Solution | 1% Molar | 59.9 | 49.8 |
| (Low Conc) | Acid | (1) | Ratio | | |
| AntiOx ‘G’ | Dihydrolipoic | Lipid Solution | 5% Molar | 61.7 | 48.3 |
| (High Conc) | Acid | (1) | Ratio | | |

The following formulations also contained 20 mM citrate in the nucleic acid solution: AntiOx ‘C’ (Method 2); AntiOx ‘G’ (Low Conc); and AntiOx ‘G’ (High Conc).

### Antioxidant Study 3

With EDTA selected as the lead antioxidant for use in SNALP, a subsequent panel of nine EDTA formulations was manufactured to examine effects such as EDTA concentration, type (Na vs. Ca), and method of incorporation into SNALP.

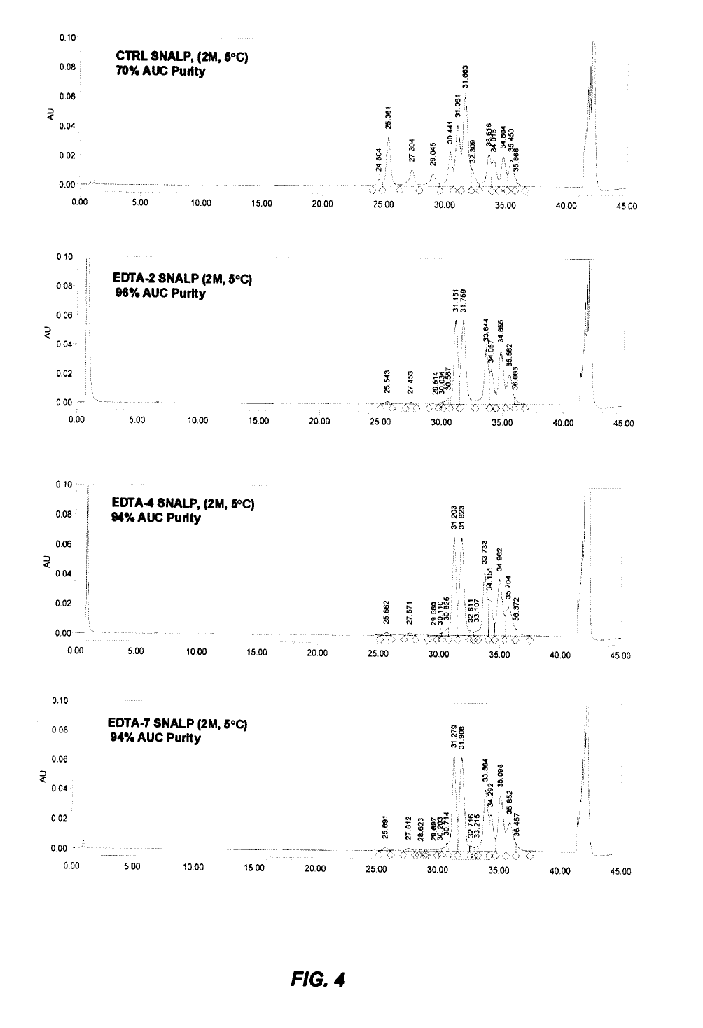

IPRP-HPLC of SNALP after 2 months storage at both 5° C. and 37° C. is summarized in Table 5, with Table 6 providing a key of the antioxidants and concentrations used during the SNALP formulation process. As with the previous study, at 5° C. the siRNA payload was effectively preserved in all tested EDTA formulations, revealing EDTA to be an extremely robust antioxidant when used in SNALP. More impressively, after 2 months storage at 37° C., the majority of the payload was still intact in all EDTA formulations, compared with the control in which more than half of it had degraded. Sample traces are displayed in FIG. 4.

TABLE 5
IPRP-HPLC data showing duplex purity after
2 months at 5° C. or 37° C.

| | 2 Months, 5° C. | 2 Months, 37° C. |
|---|---|---|
| | IPRP-HPLC, AUC % | IPRP-HPLC, AUC % |
| Description | Purity | Purity |
| ApoB siRNA Std | 97.6 | 95.4 |
| (Stored at −20° C.) | | |
| CTRL SNALP | 70.0 | 40.4 |
| EDTA-2 SNALP | 95.9 | 86.1 |
| EDTA-3 SNALP | 94.3 | 87.0 |
| EDTA-4 SNALP | 94.2 | 88.9 |
| EDTA-5 SNALP | 94.6 | 90.1 |
| EDTA-6 SNALP | 94.6 | 89.6 |
| EDTA-7 SNALP | 93.6 | 87.1 |
| EDTA-8 SNALP | 96.1 | 84.6 |
| EDTA-9 SNALP | 93.5 | 89.8 |
| EDTA-10 SNALP | 93.0 | 87.4 |

TABLE 6
Antioxidant codes for the EDTA SNALP formulations shown in Tables 4 and 5.

| | Nucleic Acid | | |
|---|---|---|---|
| | Solution (2) | Dilution buffer (3) | Exchange Buffer (4) |
| EDTA-1a | 20 mM Na-EDTA | PBS + 20 mM Na-EDTA | PBS + 20 mM Na-EDTA |
| EDTA- | 100 mM Na-EDTA | PBS + 100 mM Na-EDTA | PBS + 100 mM Na-EDTA |
| 1b | | | |
| EDTA-2 | 20 mM Na-EDTA | PBS + 20 mM Na-EDTA | PBS + 20 mM Na-EDTA |
| EDTA-3 | 20 mM Na-EDTA | PBS + 5 mM Calcium | PBS + 5 mM Calcium |
| | | EDTA | EDTA |
| EDTA-4 | 20 mM Na-EDTA | PBS + 20 mM Calcium | PBS + 20 mM Calcium |
| | | EDTA | EDTA |
| EDTA-5 | 20 mM Na-EDTA | PBS + 100 mM Calcium | PBS + 100 mM Calcium |
| | | EDTA | EDTA |
| EDTA-6 | 100 mM Na-EDTA | PBS + 100 mM Calcium | PBS + 100 mM Calcium |
| | | EDTA | EDTA |
| EDTA-7 | 20 mM Na-EDTA | PBS | PBS |
| EDTA-8 | 20 mM citrate | PBS + 20 mM Na-EDTA | PBS |
| EDTA-9 | 20 mM citrate | PBS | PBS + 20 mM Na-EDTA |
| EDTA-10 | 20 mM Na-EDTA | PBS + 20 mM Na-EDTA | PBS |

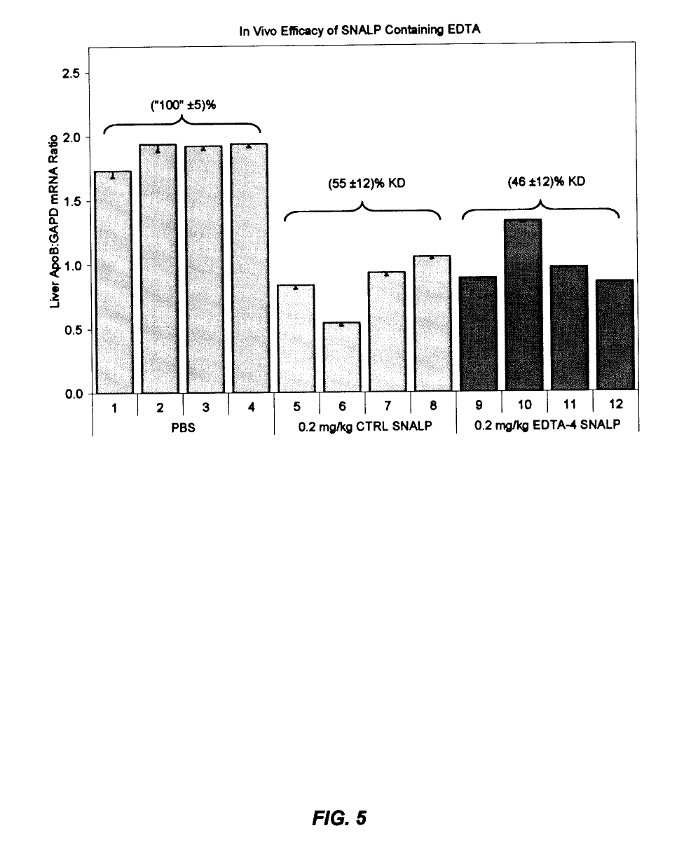

The EDTA-4 SNALP formulation was selected as a putative lead formulation. Concurrent with stability data collection, in vivo studies were conducted to evaluate biological performance of EDTA-4 SNALP in relation to the control SNALP. In vivo drug activity assessment in a mouse model demonstrated that there was no significant difference in efficacy between EDTA-4 SNALP and the control SNALP (Student's T-test p=0.32); both formulations exhibited the expected gene silencing effect shown in FIG. 5.

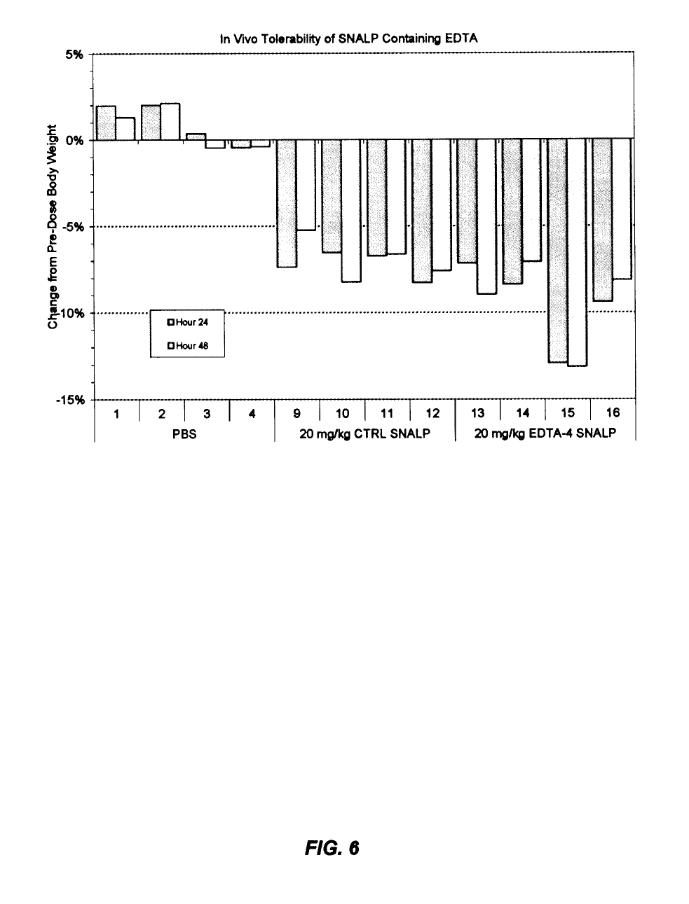

Tolerability of EDTA-4 SNALP treatment in mice was studied using an siRNA dosage 100-fold greater than the dosage used for efficacy testing. This dosage was deliberately selected to compare the effect of EDTA-4 SNALP against the liver toxicity known to result from high-dose control SNALP treatment. The following parameters were assessed: clinical signs; body weight (FIG. 6); and clinical chemistry at 48 h (Table 7). Results show that high dose EDTA-4 SNALP treatment results in a liver toxicity profile that is comparable to control SNALP treatment.

TABLE 7
Clinical chemistry following EDTA-4 SNALP treatment.

| | | ALT | AST | SDH | AlkPhos | Bilirubin | GGT | BUN | Creat | TP | Alb | Glob |
|---|---|---|---|---|---|---|---|---|---|---|---|---|
| | | iu/l | iu/l | iu/l | iu/l | umol/l | iu/l | mmol/l | umol/l | g/l | g/l | g/l |
| PBS | 1 | 35 | 34 | 24.4 | 166 | 4 | 1 | 10.9 | 33.4 | 48 | 31.7 | 16 |
| | 2 | 32 | 52 | 24.0 | 146 | 3 | 4 | 10.8 | 35.0 | 49 | 33.3 | 16 |
| | 3 | 41 | 55 | 23.3 | 147 | 5 | 2 | 11.1 | 32.2 | 48 | 32.6 | 15 |
| | 4 | 36 | 60 | 21.8 | 151 | 4 | 4 | 12.2 | 33.9 | 47 | 31.4 | 16 |
| 20 mg/kg | 5 | 4157 | 4586 | >910.5 | 186 | 4 | 4 | 10.5 | 25.9 | 47 | 29.7 | 17 |
| CTRL | 6 | 4678 | 5221 | 1310.4 | 287 | 6 | 6 | 8.1 | NSQ | 46 | NSQ | NSQ |
| SNALP | 7 | 2113 | 2415 | 193.8 | 219 | 4 | 5 | 9.0 | NSQ | 45 | NSQ | NSQ |
| | 8 | 6554 | 8001 | 696.6 | 253 | 4 | 5 | 8.2 | 29.6 | 45 | 27.8 | 17 |
| 20 mg/kg | 9 | 4949 | 4521 | 1473.5 | 357 | 5 | 9 | 6.2 | 23.2 | 44 | 25.4 | 19 |
| EDTA-4 | 10 | 2388 | 2411 | 627.7 | 313 | 4 | 5 | 8.2 | NSQ | 45 | 28.7 | 16 |
| SNALP | 11 | 3764 | 4877 | 1048.8 | 311 | 4 | 9 | 7.7 | NSQ | 45 | NSQ | NSQ |
| | 12 | 3981 | 4799 | 1094.4 | 267 | 2 | 6 | 8.8 | 22.1 | 42 | 24.3 | 18 |

BALB/c mice (n = 4) were administered SNALP as bolus tail vein injections at an siRNA dosage of 20 mg/kg. Blood was collected via cardiac puncture at 48 h for analysis.

### Antioxidant Study 4

It was apparent from Study 3 that potent antioxidant activity could be maintained while further restricting EDTA incorporation below the levels that were used to prepare EDTA-4 SNALP. EDTA-7 SNALP was therefore selected as another putative lead formulation. Drug activity data for EDTA-7 SNALP were collected in cultured cells and whole animal models, along with rodent toxicity profiles.

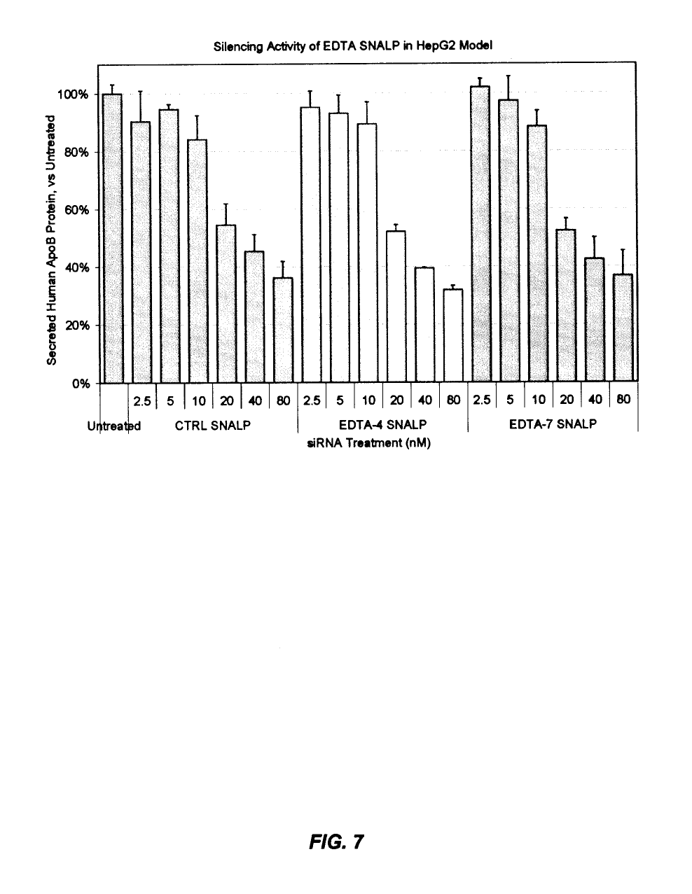

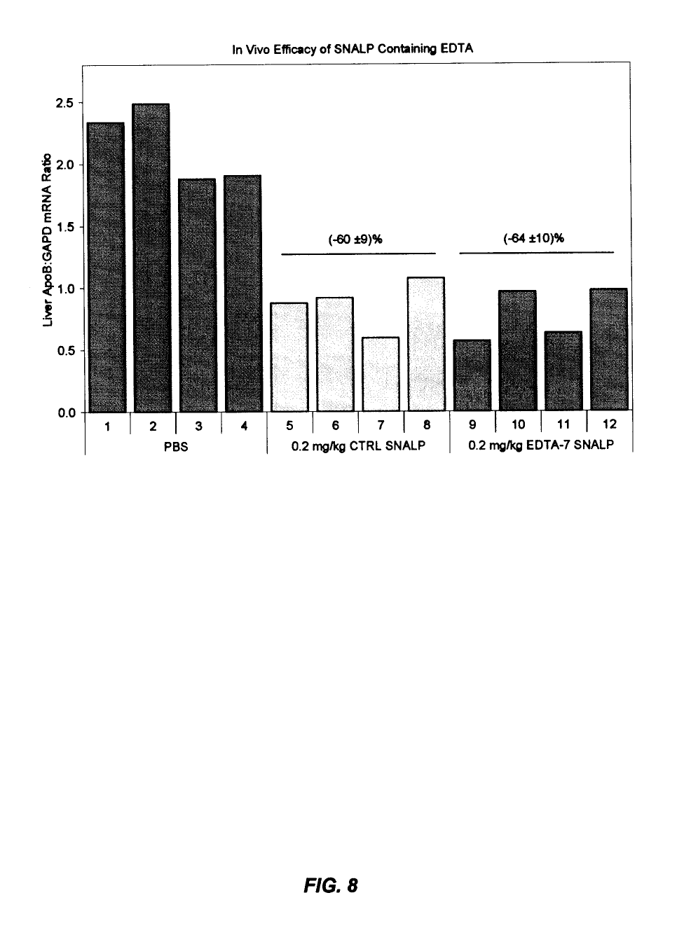

Drug activity for EDTA-4 and EDTA-7 SNALP were compared in a HepG2 cell model. FIG. 7 indicates no appreciable difference between gene silencing ability of EDTA-4, EDTA-7, and control SNALP formulations. As with EDTA-4 SNALP, the drug activity of EDTA-7 SNALP was verified in an in vivo mouse model (FIG. 8), which indicated that there was no significant change in strength of gene silencing in relation to control SNALP (Student's T-test p=0.60).

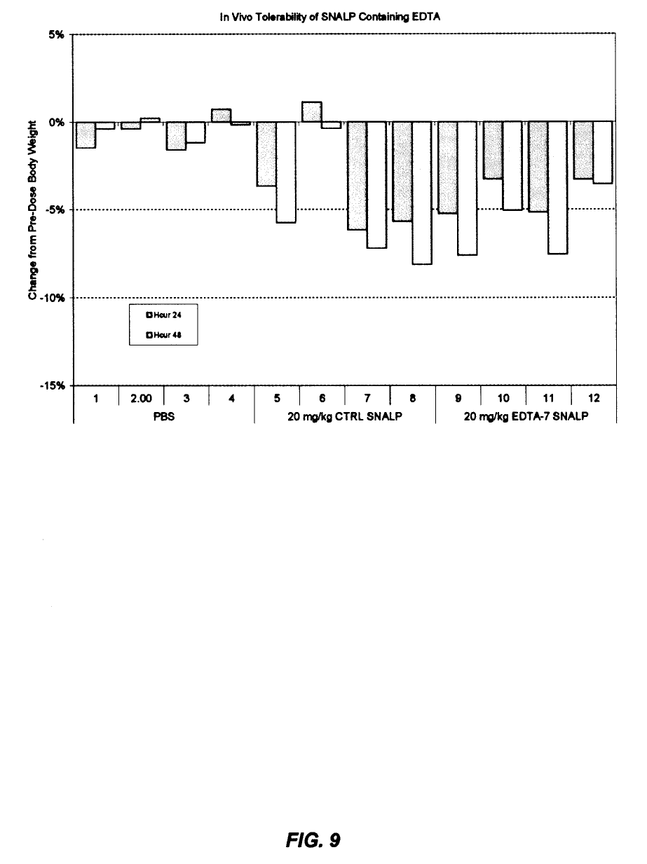

Similar to the tolerability testing performed on EDTA-4 SNALP (direct comparison to control SNALP at a dose that is expected to induce liver toxicity), mouse responses to 20 mg/kg EDTA-7 SNALP treatment were assessed over a 48 h course of study with regards to clinical signs, daily body weight (FIG. 9), as well as hematology and clinical chemistry at 48 h (Table 8). Data collected for EDTA-7 SNALP is very similar to results for control SNALP as well as EDTA-4 SNALP.

TABLE 8
Hematology and clinical chemistry following EDTA-7 SNALP treatment.

| | | WBC | Neut | Lymph | Mono | Eosino | Baso | Platelets | MPV | RBC | Hg | Hcrit |
|---|---|---|---|---|---|---|---|---|---|---|---|---|
| | | 10e9/L | 10e9/L | 10e9/L | 10e9/L | 10e9/L | 10e9/L | 10e9/L | fl | 10e9/L | g/L | L/L |
| PBS | 1 | 3.09 | 0.49 | 2.48 | 0.09 | 0.02 | 0.02 | 1240 | 4.05 | 9.56 | 143.0 | 0.458 |
| | 2 | 3.87 | 0.61 | 3.12 | 0.07 | 0.02 | 0.06 | 1089 | 4.04 | 9.19 | 145.0 | 0.450 |
| 20 mg/kg | 5 | 2.72 | 0.53 | 1.77 | 0.30 | 0.01 | 0.11 | 1009 | 4.43 | 9.69 | 151.0 | 0.476 |
| CTRL | 6 | 3.19 | 1.35 | 1.37 | 0.38 | 0.06 | 0.03 | 862 | 5.05 | 10.80 | 162.0 | 0.520 |
| SNALP | | | | | | | | | | | | |
| 20 mg/kg | 9 | 3.17 | 1.45 | 1.62 | 0.06 | 0.02 | 0.03 | 809 | 5.25 | 11.00 | 170.0 | 0.526 |
| EDTA-7 | 10 | 5.14 | 1.83 | 3.11 | 0.11 | 0.00 | 0.09 | 863 | 5.69 | 10.20 | 162.0 | 0.493 |
| SNALP | | | | | | | | | | | | |

| | | ALT | AST | SDH | AlkPhos | Bilirubin | GGT | BUN | Creat | TP | Alb | Glob |
|---|---|---|---|---|---|---|---|---|---|---|---|---|
| | | iu/l | iu/l | iu/l | iu/l | umol/l | iu/l | mmol/l | umol/l | g/l | g/l | g/l |
| PBS | 3 | 35 | 53 | 23.3 | 134 | 5 | 3 | 11.5 | 30.8 | 43 | 32.1 | 11 |
| | 4 | 37 | 49 | 23.1 | 131 | 4 | 6 | 10.3 | 27.5 | 44 | 31.6 | 12 |
| 20 mg/kg | 7 | 1870 | 2316 | 150.5 | 154 | 4 | 5 | 11.0 | 24.7 | 44 | 29.9 | 14 |
| CTRL | 8 | 3493 | 3476 | 64.2 | 238 | 5 | 8 | 5.9 | 21.3 | 40 | 27.2 | 13 |
| SNALP | | | | | | | | | | | | |
| 20 mg/kg | 11 | 4987 | 5240 | 21.0 | 297 | 6 | 5 | 5.9 | 23.5 | 41 | 27.2 | 14 |
| EDTA-7 | 12 | 4119 | 4493 | 81.4 | 192 | 5 | 1 | 7.9 | 23.5 | 44 | 28.5 | 16 |
| SNALP | | | | | | | | | | | | |

BALB/c mice (n = 4) were administered SNALP as bolus tail vein injections at an siRNA dosage of 20 mg/kg. Blood was collected at 48 h for analysis (n = 2 for hematology, n = 2 for chemistry).

Finally, tolerability of EDTA-4 and EDTA-7 SNALP was also assessed in rats. This study had a focused objective of determining whether inclusion of EDTA caused SNALP to be less well-tolerated in this second rodent model. For this purpose, a control SNALP dose considered to be just above the NOAEL (not causing severe toxicity) was selected. In addition to monitoring clinical signs and body weight, 24 h hematology, clinical chemistry, coagulation factors and major organ weights were measured for each individual animal.

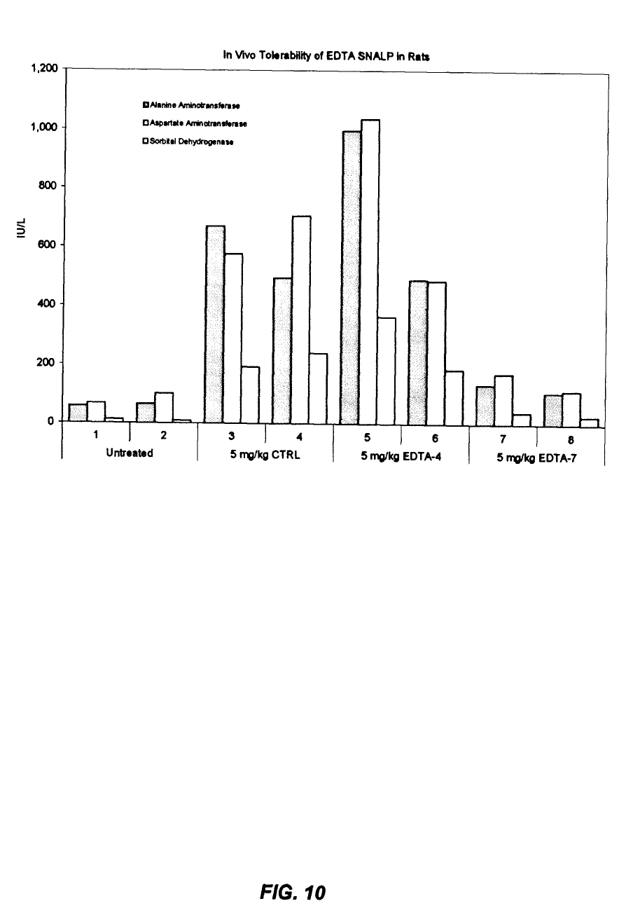

At an siRNA dosage of 5 mg/kg, rats appeared to tolerate all SNALP treatments well, with no clinical signs of adverse response over the 24 hour course of study. Results illustrated in FIG. 10 and summarized in Tables 9 & 10 show that both EDTA SNALP formulations appeared at least as well-tolerated as control SNALP. In fact, the data shows that EDTA-7 SNALP is somewhat better tolerated that control SNALP, as evidenced by body weights, liver enzymes, platelets, and activated partial thromboplastin times.

TABLE 9
Rat body and organ weights following treatment
with EDTA-4 or EDTA-7 SNALP.

| | | 24 h | Liver | Spleen | Heart | Kidneys | Lungs |
|---|---|---|---|---|---|---|---|
| | | BW | as % | as % | as % | as % | as % |
| | | Change | BW | BW | BW | BW | BW |
| Untreated | 1 | 0.7% | 4.1% | 0.22% | 0.36% | 0.71% | 0.46% |
| | 2 | 0.5% | 4.3% | 0.26% | 0.35% | 0.67% | 0.48% |
| 5 mg/kg | 3 | −7.3% | 3.7% | 0.27% | 0.31% | 0.68% | 0.47% |
| CTRL | 4 | −4.4% | 3.9% | 0.28% | 0.34% | 0.71% | 0.49% |
| SNALP | | | | | | | |
| 5 mg/kg | 5 | −6.0% | 3.4% | 0.27% | 0.32% | 0.63% | 0.46% |
| EDTA-4 | 6 | −4.3% | 3.8% | 0.28% | 0.33% | 0.62% | 0.43% |
| SNALP | | | | | | | |
| 5 mg/kg | 7 | −1.8% | 4.5% | 0.28% | 0.35% | 0.76% | 0.45% |
| EDTA-7 | 8 | −2.3% | 4.2% | 0.25% | 0.35% | 0.72% | 0.47% |
| SNALP | | | | | | | |

Male Sprague-Dawley rats were administered SNALP as bolus tail vein injections at an siRNA dosage of 5 mg/kg. BW = body weight. Organ weights were collected at 24 h and are expressed as percentage of pre-dose body weight.

TABLE 10
Rat clinical chemistry, hematology and coagulation values
following treatment with EDTA-4 or EDTA-7 SNALP.

| | | ALT | AST | SDH | AlkPhos | Bilirubin | GGT | BUN | Creat | TP | Alb | Glob |
|---|---|---|---|---|---|---|---|---|---|---|---|---|
| | | iu/l | iu/l | iu/l | iu/l | umol/l | iu/l | mmol/l | umol/l | g/l | g/l | g/l |
| Untreated | 1 | 58 | 68 | 12.4 | 213 | 2 | 5 | 7.2 | 34.9 | 55 | 34.2 | 21 |
| | 2 | 65 | 101 | 9.4 | 220 | 2 | 4 | 7.8 | 35.4 | 53 | 33.9 | 19 |
| 5 mg/kg | 3 | 670 | 576 | 192.3 | 231 | 2 | 7 | 5.9 | 37.3 | 56 | 32.4 | 24 |
| CTRL | 4 | 495 | 706 | 240.4 | 264 | 2 | 13 | 2.7 | 41.1 | 55 | 32.7 | 22 |
| 5 mg/kg | 5 | 996 | 1036 | 364.3 | 320 | 3 | 5 | 6.5 | 42.2 | 53 | 31.9 | 21 |
| EDTA-4 | 6 | 490 | 486 | 185.1 | 263 | 2 | 4 | 7.5 | 49.5 | 52 | 31.8 | 20 |
| 5 mg/kg | 7 | 134 | 172 | 40.4 | 188 | 2 | 5 | 6.1 | 43.3 | 54 | 32.1 | 22 |
| EDTA-7 | 8 | 108 | 115 | 27.0 | 209 | 2 | 6 | 5.9 | 41.1 | 54 | 33.5 | 21 |

| | | WBC | Neut | Lymph | Mono | Eosino | Baso | Neut | Lymph | Mono | Eosino | Baso |
|---|---|---|---|---|---|---|---|---|---|---|---|---|
| | | 10e9/L | 10e9/L | 10e9/L | 10e9/L | 10e9/L | 10e9/L | % | % | % | % | % |
| Untreated | 1 | 14.90 | 1.09 | 12.70 | 0.54 | 0.14 | 0.43 | 7 | 85 | 4 | 1 | 3 |
| | 2 | 10.40 | 0.72 | 8.62 | 0.53 | 0.08 | 0.45 | 7 | 83 | 5 | 1 | 4 |
| 5 mg/kg | 3 | 10.90 | 6.32 | 4.36 | 0.22 | 0.00 | 0.00 | 58 | 48 | 2 | 0 | 0 |
| CTRL | 4 | 11.70 | 2.15 | 9.13 | 0.24 | 0.07 | 0.12 | 18 | 78 | 2 | 1 | 1 |
| 5 mg/kg | 5 | 13.40 | 3.81 | 9.26 | 0.13 | 0.07 | 0.12 | 28 | 69 | 1 | 1 | 1 |
| EDTA-4 | 6 | 12.30 | 3.22 | 8.60 | 0.21 | 0.13 | 0.14 | 26 | 70 | 2 | 1 | 1 |
| 5 mg/kg | 7 | 16.20 | 6.97 | 8.75 | 0.32 | 0.16 | 0.00 | 43 | 54 | 2 | 1 | 0 |
| EDTA-7 | 8 | 13.00 | 2.06 | 10.10 | 0.55 | 0.11 | 0.18 | 16 | 78 | 4 | 1 | 1 |

| | | Platelets | MPV | RBC | Hg | Hcrit | MCV | MCH | MCHC | Fib | APTT | PT |
|---|---|---|---|---|---|---|---|---|---|---|---|---|
| | | 10e9/L | fl | 10e9/L | g/L | L/L | fl | pg | f/L | g/L | sec | sec |
| Untreated | 1 | 1029 | 5.85 | 7.85 | 155.0 | 0.466 | 59.4 | 19.8 | 333 | 1 | 20.2 | 19.6 |
| | 2 | 1152 | 5.04 | 7.19 | 143.0 | 0.417 | 58.0 | 19.9 | 343 | 4 | 20.4 | 19.4 |
| 5 mg/kg | 3 | 864 | 5.66 | 8.46 | 171.0 | 0.502 | 59.3 | 20.3 | 342 | 2 | 28.7 | 18.1 |
| CTRL | 4 | 1010 | 5.95 | 8.04 | 159.0 | 0.467 | 58.0 | 19.8 | 341 | 2 | 25.8 | 17.3 |
| 5 mg/kg | 5 | 859 | 6.14 | 7.72 | 154.0 | 0.453 | 58.7 | 19.9 | 339 | 4 | 26.4 | 18.7 |
| EDTA-4 | 6 | 848 | 5.53 | 7.53 | 150.0 | 0.438 | 58.1 | 19.9 | 342 | 4 | 23.0 | 17.5 |
| 5 mg/kg | 7 | 1021 | 5.32 | 7.88 | 150.0 | 0.441 | 56.0 | 19.1 | 340 | 2 | 21.4 | 19.4 |
| EDTA-7 | 8 | 1165 | 5.49 | 7.12 | 146.0 | 0.425 | 59.7 | 20.5 | 343 | 2 | 21.6 | 17.6 |

Male Sprague-Dawley rats were administered SNALP as bolus tail vein injections at an siRNA dosage of 5 mg/kg. Blood was collected at 24 h for analysis.

### CONCLUSION

IPRP-HPLC data revealed that EDTA-formulated SNALP containing siRNA have an excellent stability profile for at least 3 months at 5° C. Even at elevated temperatures, the siRNA is effectively preserved for several months. IPRP-HPLC data also revealed that a higher concentration of citrate (e.g., 100 mM citrate), when added to all of the aqueous buffers during the SNALP formulation process, was particularly effective at stabilizing the siRNA payload at 5° C. and ambient temperature. These findings are in stark contrast to the extensive payload conversion observed with other formulations.

Furthermore, EDTA incorporation does not have any negative impact on gene silencing as assessed in vitro in a HepG2 cell model as well as in vivo in a BALB/c mouse model. High dose testing in mice showed that EDTA incorporation does not substantially alter the liver toxicity profile of SNALP. In rats, the EDTA-7 SNALP formulation appears at least as well-tolerated as control SNALP.

### Example 2

### SNALP Stability Study: Comparison of EDTA Versus Low Citrate Concentration

This example demonstrates that low EDTA concentrations (e.g., 20 mM) prevented the oxidative degradation of both the lipid and nucleic acid components of SNALP, whereas a similar protective effect was not observed with low citrate concentrations (e.g., 20 mM).

The nucleic acid payload used in this study is an siRNA having phosphorothioate (PS) linkages. The SNALP formulation used in this study comprises the following lipid composition: PEG2000-C-DMA (1.43 mol %); DLinDMA (57.14 mol %); cholesterol (34.29 mol %); and DPPC (7.14 mol %). A pH 5 siRNA solution was prepared for mixing with an ethanolic lipid solution. SNALP formulation conditions included either the addition of 20 mM citrate or 20 mM EDTA to the SNALP formulation process. Stability of lipid and siRNA components was monitored at 5° C. for up to 9 months and at room temperature (RT) for up to 5 months.

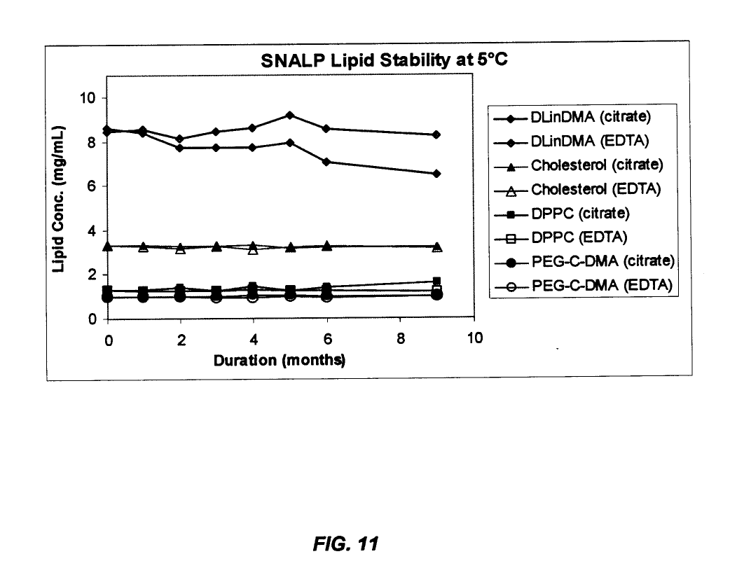

FIG. 11 shows an HPLC analysis of each of the lipid components present in SNALP over a period of 9 months at 5° C. when formulated with either 20 mM EDTA or 20 mM citrate. There was a modest reduction in DLinDMA concentration observed with the citrate SNALP formulation. However, all lipid components (including DLinDMA) were stable in the EDTA SNALP formulation.

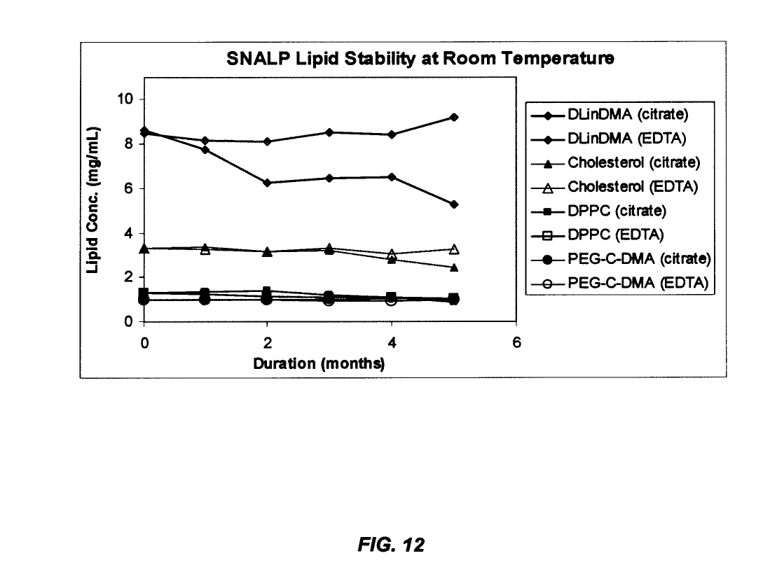

FIG. 12 shows an HPLC analysis of each of the lipid components present in SNALP over a period of 5 months at room temperature when formulated with either 20 mM EDTA or 20 mM citrate. There was a substantial reduction in DLinDMA concentration observed with the citrate SNALP formulation, but all lipid components (including DLinDMA) were stable in the EDTA SNALP formulation.

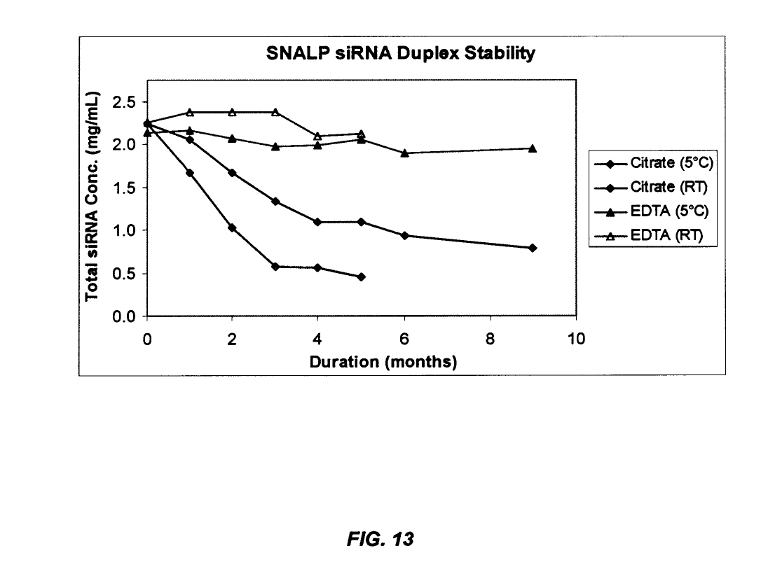

FIG. 13 shows an HPLC analysis of the siRNA component present in SNALP when formulated with either 20 mM EDTA or 20 mM citrate. There was a substantial reduction in siRNA duplex concentration observed with the citrate SNALP formulation, whereas the siRNA was stable in the EDTA SNALP formulation at both 5° C. and RT.

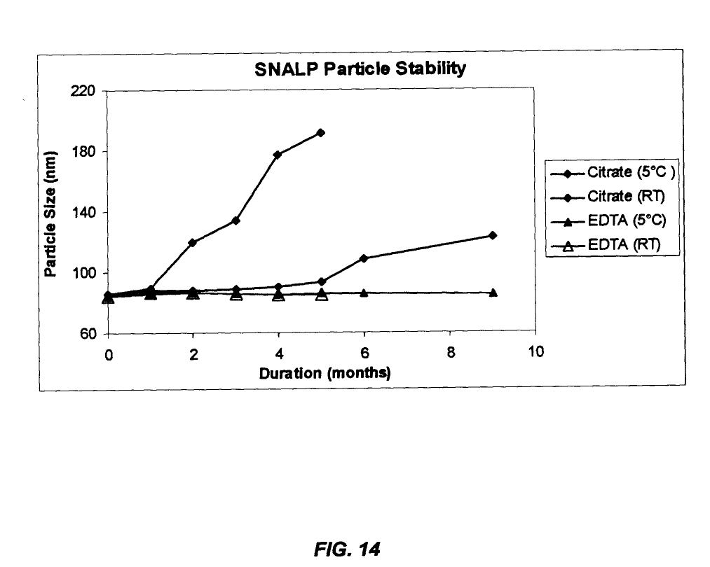

FIG. 14 shows a particle size analysis of SNALP when formulated with either 20 mM EDTA or 20 mM citrate. There was a particle size increase observed with the citrate SNALP formulation, especially at RT. In contrast, particle sizes were stable in the EDTA SNALP formulation.

Table 11 shows an siRNA purity analysis using denaturing HPLC to determine the extent of PS to phosphodiester (PO) conversion in the siRNA. The presence of EDTA in the SNALP formulation stabilized the siRNA to oxidation at both temperatures tested.

TABLE 11
EDTA prevents the desulfurization of PS-modified
siRNA encapsulated in SNALP.

| | ~Relative | % AUC | |
|---|---|---|---|
| PO Impurity | Retention Time | Citrate SNALP | EDTA SNALP |

9 months at 5° C.

| | | | |
|---|---|---|---|
| 1 | 0.47 | 9.32 | 1.10 |
| 2 | 0.67 | 5.91 | 0.92 |
| 3 | 0.76 | 6.49 | 1.13 |
| 4 | 0.85 | 10.96 | 1.36 |

| | | |
|---|---|---|
| Total PO | 32.68 | 4.51 |

5 months at RT

| | | | |
|---|---|---|---|
| 1 | 0.47 | 12.64 | 1.68 |
| 2 | 0.67 | 8.52 | 1.17 |
| 3 | 0.76 | 6.96 | 1.42 |
| 4 | 0.85 | 12.89 | 2.09 |

| | | |
|---|---|---|
| Total PO | 41.10 | 6.36 |

### Example 3

### Stabilization of SNALP Containing Polyunsaturated Lipids with Antioxidants

This example demonstrates that the metal chelator EDTA in combination with one or more additional antioxidants improves the stability of SNALP formulations containing a polyunsaturated cationic lipid such as γ-DLenDMA, MC3, MC3 Ether, or MC4 Ether. In Antioxidant Study 1, the synergistic effect of EDTA combined with another antioxidant type at two concentrations on the stability of SNALP containing γ-DLenDMA was evaluated. In Antioxidant Study 2, the synergistic effect of mixtures of EDTA, primary antioxidant, and secondary antioxidant at two concentrations of each antioxidant on the stability of SNALP containing γ-DLenDMA was evaluated using a statistical Design of Experiments model. In Antioxidant Study 3, the effect of antioxidants and mixtures thereof on stability of SNALP containing MC3, MC3 Ether, or MC4 Ether was evaluated.

### Antioxidant Study 1

SNALP Preparation:

The nucleic acid payload used in this study is an ApoB siRNA without phosphorothioate (PS) linkages at a 6:1 L/D ratio. The SNALP formulation used in this study comprises the following 1:57 lipid composition: PEG2000-C-DMA (1.43 mol %); γ-DLenDMA (57.14 mol %); cholesterol (34.29 mol %); and DPPC (7.14 mol %). The nucleic acid solution was prepared as described in Table 12, using a total lipid concentration of ˜8.1 mg/mL. For the first formulation, citrate was used instead of EDTA, but the concentrations were the same. SNALP were prepared at a 5 mg scale using 5 cc syringes with a 0.8 mm T-connector. 3.7 mL of nucleic acid solution was blended with 3.7 mL of lipid stock with direct dilution into 14.3 mL of PBS to form SNALP. Antioxidants were added to the lipid stock just before formulation. In particular, lipophilic antioxidants were incorporated at 0.1 mol % or 1.0 mol %. Formulations were then worked up by midgee hoops (4000 sec-1, 20 mL/min recirculation rate). During tangential flow ultrafiltration (TFU), SNALP were first concentrated to approximately 5 mL (total including 2 mL holdup) and diafiltered against 60 mL of PBS (12 wash volumes). The SNALP were further concentrated to <3 mL (including 2 mL holdup), discharged, and sterile filtered. SNALP were diluted to 0.5 mg/ml before the study started. Data collection was performed over a 1 month period. Analytical assays such as Malvern Nano Series Zetasizer for particle size and Varian Cary Eclipse Fluorimeter for RiboGreen analysis of encapsulation efficiency were performed on SNALP to determine their stability at t=0 and upon storage at 4° C. or room temperature (RT) for 1 month.

TABLE 12
Theoretical nucleic acid solution composition.

| | Input | | |
|---|---|---|---|
| Component | Concentration | Volume (mL) | Final Concentration |
| Nucleic acid | 14.21 mg/mL | 0.361 | 1.350 mg/mL |
| 100 mM EDTA | 100 mM | 0.760 | 20 mM |
| Water | N/A | 2.681 | N/A |

Stability of SNALP:

The SNALP formulations were stored at 4° C. and RT (22° C.). Particle sizes and encapsulation were evaluated at t=0, 1 month at 4° C., and 1 month at RT for the following antioxidants and mixtures thereof: (1) 20 mM citrate; (2) 20 mM EDTA; (3) 20 mM EDTA+0.1 mol % BHT; (4) 20 mM EDTA+1.0 mol % BHT; (5) 20 mM EDTA+0.1 mol % ascorbyl palmitate; (6) 20 mM EDTA+1.0 mol % ascorbyl palmitate; (7) 20 mM EDTA+0.1 mol % α-tocopherol; (8) 20 mM EDTA+1.0 mol % α-tocopherol; (9) 20 mM EDTA+0.1 mol % lipoic acid; and (10) 20 mM EDTA+1.0 mol % lipoic acid.

Results:

Table 13 shows the positive effect on SNALP stability with regard to both particle size and nucleic acid encapsulation when mixtures of EDTA and ascorbyl palmitate or α-tocopherol at either concentration (e.g., 0.1 mol % or 1.0 mol %) were included in the SNALP formulation process. For example, encapsulation efficiencies were greater than about 90% for each of these SNALP formulations after a period of 1 month at 4° C. In contrast, BHT at both concentrations showed detrimental results. Although the percent encapsulation was high with lipoic acid, particle sizes were about 50 nm higher than those observed with mixtures of EDTA and ascorbyl palmitate or α-tocopherol.

TABLE 13
Summary of results from Study 1 - EDTA
combined with a lipophilic antioxidant.

| Composition | | Particle size (nm) | | | Encapsulation (%) | | |
|---|---|---|---|---|---|---|---|
| | Mol | | 1 month | 1 month | | 1 month | 1 month |
| Antioxidant | % | t = 0 | 4° C. | RT | t = 0 | 4° C. | RT |
| Citrate | n/a | 91 | 152 | 171 | 97 | 81 | 36 |
| EDTA | n/a | 83 | 121 | 160 | 97 | 92 | 27 |
| EDTA + | 0.1 | 82 | 141 | 163 | 97 | 87 | 32 |
| BHT | 1.0 | 83 | 282 | 158 | 96 | 17 | 21 |
| EDTA + | 0.1 | 83 | 106 | 165 | 96 | 94 | 42 |
| Ascorbyl | 1.0 | 89 | 102 | 169 | 93 | 91 | 34 |
| palmitate | | | | | | | |
| EDTA + | 0.1 | 87 | 96 | 170 | 95 | 93 | 45 |
| Alpha- | 1.0 | 90 | 107 | 196 | 96 | 93 | 72 |
| tocopherol | | | | | | | |
| EDTA + | 0.1 | 86 | 140 | 178 | 97 | 88 | 41 |
| Lipoic acid | 1.0 | 85 | 156 | 159 | 97 | 83 | 37 |

### Antioxidant Study 2

SNALP Preparation:

The nucleic acid payload used in this study is a Luc siRNA with phosphorothioate (PS) linkages at a 6:1 L/D ratio. The SNALP formulation used in this study comprises the following 1:57 lipid composition: PEG2000-C-DMA (1.43 mol %); γ-DLenDMA (57.14 mol %); cholesterol (34.29 mol %); and DPPC (7.14 mol %). The nucleic acid solution was prepared as described in Tables 14 and 15, using a total lipid concentration of ˜8.1 mg/mL. The first four lots of SNALP had a final concentration of 20 mM EDTA and the last four lots of SNALP had a final concentration of 80 mM EDTA.

TABLE 14
Theoretical nucleic acid solution composition
for SNALP formulations 1-4.

| | Input | Volume | Final |
|---|---|---|---|
| Component | Concentration | (mL) | Concentration |
| Nucleic acid | 10.33 mg/mL | 0.497 | 1.350 mg/mL |
| 100 mM EDTA | 100 mM | 0.760 | 20 mM |
| Water | N/A | 2.545 | N/A |

TABLE 15
Theoretical nucleic acid solution composition
for SNALP formulations 5-8.

| | Input | Volume | Final |
|---|---|---|---|
| Component | Concentration | (mL) | Concentration |
| Nucleic acid | 10.33 mg/mL | 0.497 | 1.350 mg/mL |
| 160 mM EDTA | 160 mM | 1.901 | 80 mM |
| Water | N/A | 1.404 | N/A |

SNALP were prepared at a 5 mg scale using 5 cc syringes with a 0.8 mm T-connector. 3.7 mL of nucleic acid solution was blended with 3.7 mL of lipid stock with direct dilution into 14.3 mL of PBS to form SNALP. Antioxidants were added to the lipid stock just before formulation. In particular, lipophilic antioxidants were incorporated at 0.1 mol % or 1.0 mol %. Formulations were then worked up by midgee hoops (4000 sec-1, 20 mL/min recirculation rate). During tangential flow ultrafiltration (TFU), SNALP were first concentrated to approximately 5 mL (total including 2 mL holdup) and diafiltered against 60 mL of PBS (12 wash volumes). The SNALP were further concentrated to <3 mL (including 2 mL holdup), discharged, and sterile filtered. SNALP were diluted to 0.5 mg/ml before the study started. Data collection was performed over a 1 month period. Analytical assays such as Malvern Nano Series Zetasizer for particle size, Varian Cary Eclipse Fluorimeter for RiboGreen analysis of encapsulation efficiency, and DENAX siRNA analysis for phosphorothioate (PS) to phosphodiester (PO) conversion in the nucleic acid payload were performed on SNALP to determine their stability at t=0 and upon storage at 4° C. or room temperature (RT) for 1 month.

Stability of SNALP:

The SNALP formulations were stored at 4° C. and RT (22° C.). Particle sizes and percent encapsulation were evaluated at t=0, 1 month at 4° C., and 1 month at RT, while percent PO content was evaluated at t=0 and 1 month at 4° C. for the mixtures of antioxidants set forth in Table 16.

TABLE 16
Antioxidant combinations and concentrations used in this study.

| | | Ascorbyl Palmitate | Tocopherol |
|---|---|---|---|
| Formulation | EDTA (mM) | (mol %) | (mol %) |
| 1 | 20 | 0.1 | 0.1 |
| 2 | | 0.1 | 1.0 |
| 3 | | 1.0 | 0.1 |
| 4 | | 1.0 | 1.0 |
| 5 | 80 | 0.1 | 0.1 |
| 6 | | 0.1 | 1.0 |
| 7 | | 1.0 | 0.1 |
| 8 | | 1.0 | 1.0 |

Results:

Table 17 shows that Formulation 1 (i.e., mixture of 20 mM EDTA+0.1 mol % ascorbyl palmitate+0.1 mol % α-tocopherol), Formulation 3 (i.e., mixture of 20 mM EDTA+1.0 mol % ascorbyl palmitate+0.1 mol % α-tocopherol), Formulation 5 (i.e., mixture of 80 mM EDTA+0.1 mol % ascorbyl palmitate+0.1 mol % α-tocopherol), and Formulation 7 (i.e., mixture of 80 mM EDTA+1.0 mol % ascorbyl palmitate+0.1 mol % α-tocopherol) demonstrated significant improvement in stability of SNALP. In particular, these formulations exhibited little to no change in particle size, percent encapsulation, and percent PO content over a 1 month period at both 4° C. and room temperature (RT). For example, encapsulation efficiencies were greater than about 90% (e.g., greater than about 95%) for each of these SNALP formulations after 1 month at both 4° C. and RT. In addition, particle sizes were less than about 100 nm (e.g., between about 70 nm to about 90 nm) for each of these SNALP formulations after 1 month at both 4° C. and RT. Furthermore, there was little change in the percent PS to PO conversion observed with the siRNA payload based upon the PO content of the formulations after 1 month at 4° C.

TABLE 17
Summary of results from Study 2 - EDTA combined with 2 lipophilic
antioxidants: a primary antioxidant and a secondary antioxidant.

| Composition | | | Particle size (nm) | | | Encapsulation (%) | | | PO content (%) | |
|---|---|---|---|---|---|---|---|---|---|---|
| Ascorbyl | Alpha- | | | 1 | 1 | | 1 | 1 | | 1 |
| Palmitate | Tocopherol | EDTA | | month | month | | month | month | | month |
| (mol %) | (mol %) | (mM) | t = 0 | 4 C. | RT | t = 0 | 4 C. | RT | t = 0 | 4 C. |
| 0.1 | 0.1 | 20 | 79 | 81 | 83 | 99 | 98 | 94 | 11.7 | 18.6 |
| 0.1 | 1.0 | 20 | 80 | 91 | 143 | 98 | 98 | 89 | 18.4 | 34.1 |
| 1.0 | 0.1 | 20 | 80 | 80 | 82 | 98 | 97 | 95 | 8.7 | 10.2 |
| 1.0 | 1.0 | 20 | 81 | 84 | 118 | 98 | 98 | 92 | 8.3 | 15.8 |
| 0.1 | 0.1 | 80 | 76 | 82 | 78 | 98 | 98 | 96 | 10.2 | 14.9 |
| 0.1 | 1.0 | 80 | 81 | 95 | 114 | 98 | 96 | 91 | 14.8 | 26.7 |
| 1.0 | 0.1 | 80 | 84 | 88 | 85 | 98 | 97 | 95 | 7.8 | 9.9 |
| 1.0 | 1.0 | 80 | 84 | 89 | 103 | 98 | 97 | 93 | 7.7 | 13.3 |

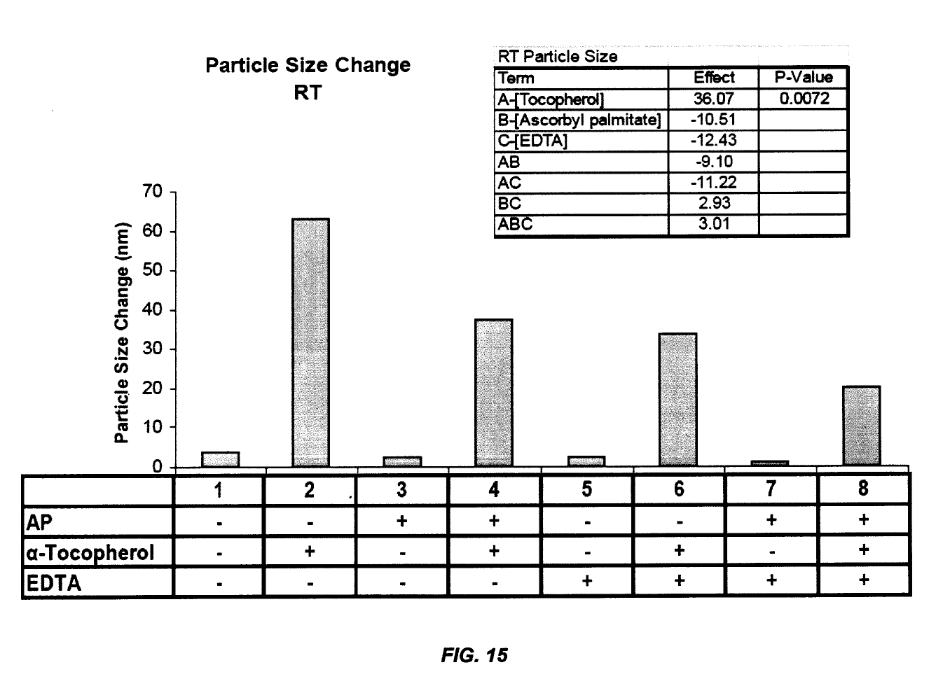

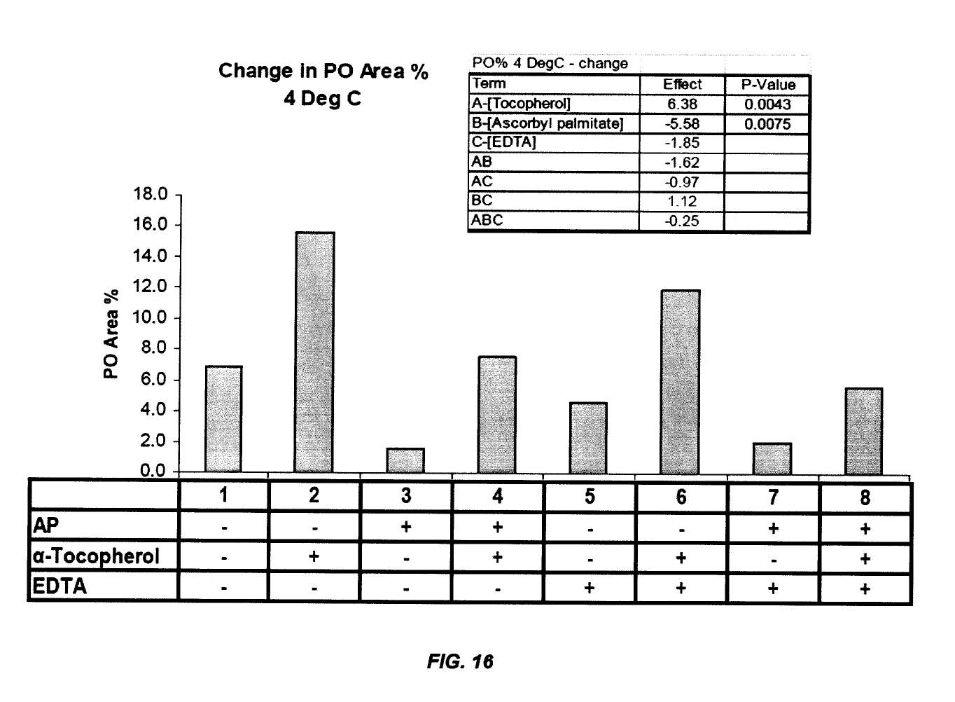

A statistical Design of Experiments (DOE) analysis was applied to the formulations to determine significance in variation caused by the different conditions. FIGS. 15-16 show the results for Formulations 1-8 with regard to particle size and percent PO content over a 1 month period. For ascorbyl palmitate (AP) and α-tocopherol: “−” means 0.1 mol %; “+” means 1.0 mol %. For EDTA: “−” means 20 mM EDTA; “+” means 80 mM EDTA. The table at the top of each figure shows statistical significance.

### Antioxidant Study 3

SNALP Preparation:

The SNALP formulation used in this study comprises the following 1:57 lipid composition: PEG2000-C-DMA (1.43 mol %); polyunsaturated cationic lipid (57.14 mol %); cholesterol (34.29 mol %); and DPPC (7.14 mol %). Lipid stocks (1:57 lipid composition) were prepared with either MC3, MC3 Ether, or MC4 Ether. For batches containing antioxidants, the lipid stocks were prepared on the day of formulation. siRNA solutions were prepared with either 20 mM EDTA or 20 mM citrate, pH 5. SNALP formulations were prepared at the 15 mg input siRNA scale by automated syringe press (lipobot). The formulations are listed in Table 18. The formulations were worked-up by tangential flow ultrafiltration (TFU) using hollow fiber cartridges (midgee hoops). SNALP stability was evaluated at 0.5 mg/mL siRNA. 0.5 mL of SNALP was stored in cryogenic vials at 4° C., RT (22° C.), and 40° C.

TABLE 18
List of SNALP formulations prepared.

| | siRNA buffer | Antioxidants |
|---|---|---|
| Description | condition | in lipid stock |
| 1:57 MC3 | 20 mM EDTA | none |
| 1:57 MC3 Ether | 20 mM EDTA | none |
| 1:57 MC4 Ether | 20 mM EDTA | none |
| 1:57 MC3 | 20 mM citrate | none |
| 1:57 MC3 Ether | 20 mM citrate | none |
| 1:57 MC4 Ether | 20 mM citrate | none |
| 1:57 MC3 | 20 mM EDTA | 0.1% alpha-tocopherol, |
| | | 1% ascorbyl palmitate |
| 1:57 MC3 Ether | 20 mM EDTA | 0.1% alpha-tocopherol, |
| | | 1% ascorbyl palmitate |
| 1:57 MC4 Ether | 20 mM EDTA | 0.1% alpha-tocopherol, |
| | | 1% ascorbyl palmitate |

Results:

Samples were removed after 2 weeks of storage at room temperature (RT) or 40° C. (accelerated conditions). Samples were analyzed for particle size, encapsulation efficiency, lipid content and purity, and siRNA content and purity. Particle size measurements were obtained using a Malvern Zetasizer instrument and encapsulation efficiency was determined using a RiboGreen fluorometric assay. Lipid content and purity were analyzed by reverse phase HPLC. siRNA content and purity were analyzed by denaturing anion-exchange HPLC (DENAX).

The particle size and siRNA encapsulation data for the formulations are presented in Tables 19 and 20, respectively. The particle sizes were stable at both storage temperatures with the exception of the MC4 Ether 20 mM citrate SNALP stored at 40° C., which showed a modest size increase. No significant changes in encapsulation efficiency were observed.

TABLE 19
Particle Size.

| Composition | | Particle size (nm) | | |
|---|---|---|---|---|
| Cationic | | | 2 weeks | 2 weeks |
| lipid | Condition | t = 0 | RT | 40° C. |
| MC3 | Citrate | 88 | 89 | 91 |
| | EDTA | 86 | 86 | 85 |
| | EDTA + alpha-tocopherol + | 84 | 84 | 85 |
| | ascorbyl palmitate | | | |
| MC3 Ether | Citrate | 89 | 90 | 90 |
| | EDTA | 84 | 88 | 86 |
| | EDTA + alpha-tocopherol + | 85 | 85 | 86 |
| | ascorbyl palmitate | | | |
| MC4 Ether | Citrate | 92 | 92 | 100 |
| | EDTA | 96 | 96 | 96 |
| | EDTA + alpha-tocopherol + | 98 | 95 | 98 |
| | ascorbyl palmitate | | | |

TABLE 20
Encapsulation.

| Composition | | Encapsulation (%) | | |
|---|---|---|---|---|
| Cationic | | | 2 weeks | |
| lipid | Condition | t = 0 | RT | 2 weeks 40° C. |
| MC3 | Citrate | 99 | 98 | 98 |
| | EDTA | 99 | 99 | 99 |
| | EDTA + alpha- | 99 | 98 | 98 |
| | tocopherol + ascorbyl | | | |
| | palmitate | | | |
| MC3 Ether | Citrate | 99 | 99 | 99 |
| | EDTA | 99 | 99 | 99 |
| | EDTA + alpha- | 98 | 98 | 98 |
| | tocopherol + ascorbyl | | | |
| | palmitate | | | |
| MC4 Ether | Citrate | 99 | 99 | 98 |
| | EDTA | 99 | 99 | 99 |
| | EDTA + alpha- | 99 | 99 | 99 |
| | tocopherol + ascorbyl | | | |
| | palmitate | | | |

DENAX HPLC analysis assessing siRNA content and purity (% AUC) is shown in Tables 21 and 22. The SNALP formulations prepared with EDTA or EDTA combined with additional antioxidants tested with significantly higher siRNA content and purity compared to the formulations containing 20 mM citrate after 2 weeks of storage under the accelerated conditions. At room temperature (RT), the antioxidant mixture improved formulation stability over the EDTA condition. The same trends in siRNA stability were observed regardless of which cationic lipid was present in the formulation.

TABLE 21
siRNA Content by DENAX.

| Composition | | siRNA content (mg/mL) | | |
|---|---|---|---|---|
| Cationic | | | | 2 weeks |
| lipid | Condition | t = 0 | 2 weeks RT | 40° C. |
| MC3 | Citrate | 0.425 | 0.347 | 0.195 |
| | EDTA | 0.430 | 0.364 | 0.381 |
| | EDTA + alpha- | 0.424 | 0.411 | 0.350 |
| | tocopherol + ascorbyl | | | |
| | palmitate | | | |
| MC3 Ether | Citrate | 0.428 | 0.368 | 0.200 |
| | EDTA | 0.423 | 0.398 | 0.367 |
| | EDTA + alpha- | 0.449 | 0.426 | 0.360 |
| | tocopherol + ascorbyl | | | |
| | palmitate | | | |
| MC4 Ether | Citrate | 0.429 | 0.329 | 0.153 |
| | EDTA | 0.419 | 0.379 | 0.355 |
| | EDTA + alpha- | 0.448 | 0.412 | 0.316 |
| | tocopherol + ascorbyl | | | |
| | palmitate | | | |

TABLE 22
siRNA Purity by DENAX.

| Composition | | siRNA purity (% AUC) | | |
|---|---|---|---|---|
| Cationic | | | | 2 weeks |
| lipid | Condition | t = 0 | 2 weeks RT | 40° C. |
| MC3 | Citrate | 90.8 | 76.9 | 48.4 |
| | EDTA | 91.2 | 83.3 | 79.1 |
| | EDTA + alpha- | 92.0 | 89.8 | 79.8 |
| | tocopherol + ascorbyl | | | |
| | palmitate | | | |
| MC3 Ether | Citrate | 91.8 | 80.1 | 55.7 |
| | EDTA | 91.6 | 86.3 | 83.9 |
| | EDTA + alpha- | 92.3 | 90.4 | 79.4 |
| | tocopherol + ascorbyl | | | |
| | palmitate | | | |
| MC4 Ether | Citrate | 91.3 | 77.3 | 44.1 |
| | EDTA | 91.2 | 86.0 | 80.9 |
| | EDTA + alpha- | 92.0 | 88.1 | 76.5 |
| | tocopherol + ascorbyl | | | |
| | palmitate | | | |

| | |
|---|---|
| STANDARD | 92.7 |

Reverse phase HPLC analysis was performed to assess the level of lipid degradation. The results are shown Tables 23-25. No significant changes in lipid content were observed for formulations stored at room temperature. However, the 20 mM citrate formulations stored at 40° C. showed a drop in cationic lipid content, with MC4 Ether demonstrating the largest drop from about 58 mol % to about 52 mol %. The formulations containing EDTA or EDTA combined with additional antioxidants had no significant change in lipid content at either storage temperature. The same trends in lipid stability were observed regardless of which cationic lipid was present in the formulation.

TABLE 23
MC3 SNALP Lipid Stability by HPLC.

| | | t = 0 | | 2 weeks at RT | | 2 weeks at 40° C. | |
|---|---|---|---|---|---|---|---|
| | | [Lipid] | | [Lipid] | | [Lipid] | |
| Sample | Lipid | (mg/mL) | Mol % | (mg/mL) | Mol % | (mg/mL) | Mol % |
| MC3 citrate | Cholesterol | 0.72 | 33.8% | 0.66 | 33.6% | 0.66 | 35.5% |
| | PEG2000-C-DMA | 0.22 | 1.5% | 0.17 | 1.3% | 0.18 | 1.3% |
| | DPPC | 0.31 | 7.5% | 0.26 | 6.9% | 0.26 | 7.3% |
| | MC3 | 2.03 | 57.2% | 1.90 | 58.2% | 1.72 | 55.9% |
| MC3 EDTA | Cholesterol | 0.64 | 33.0% | 0.66 | 33.4% | 0.67 | 33.1% |
| | PEG2000-C-DMA | 0.20 | 1.5% | 0.17 | 1.2% | 0.18 | 1.2% |
| | DPPC | 0.27 | 7.3% | 0.26 | 6.8% | 0.26 | 6.7% |
| | MC3 | 1.89 | 58.3% | 1.92 | 58.5% | 1.98 | 59.0% |
| MC3 EDTA + | Cholesterol | 0.66 | 32.7% | 0.59 | 33.4% | 0.59 | 33.3% |
| alpha-tocopherol + | PEG2000-C-DMA | 0.21 | 1.5% | 0.16 | 1.3% | 0.16 | 1.3% |
| ascorbyl | DPPC | 0.29 | 7.6% | 0.23 | 7.0% | 0.23 | 6.8% |
| palmitate | MC3 | 1.95 | 58.2% | 1.70 | 58.3% | 1.72 | 58.7% |

TABLE 24
MC3 Ether SNALP Lipid Stability by HPLC.

| | | t = 0 | | 2 weeks at RT | | 2 weeks at 40° C. | |
|---|---|---|---|---|---|---|---|
| | | [Lipid] | | [Lipid] | | [Lipid] | |
| Sample | Lipid | (mg/mL) | Mol % | (mg/mL) | Mol % | (mg/mL) | Mol % |
| MC3 Ether | Cholesterol | 0.72 | 34.8% | 0.65 | 34.9% | 0.69 | 37.8% |
| citrate | PEG2000-C-DMA | 0.22 | 1.5% | 0.18 | 1.4% | 0.18 | 1.4% |
| | DPPC | 0.29 | 7.5% | 0.25 | 7.2% | 0.25 | 7.2% |
| | MC3 Ether | 1.84 | 56.3% | 1.66 | 56.5% | 1.56 | 53.6% |
| MC3 Ether | Cholesterol | 0.66 | 34.7% | 0.62 | 33.9% | 0.62 | 35.0% |
| EDTA | PEG2000-C-DMA | 0.21 | 1.6% | 0.18 | 1.4% | 0.18 | 1.4% |
| | DPPC | 0.28 | 7.7% | 0.25 | 7.3% | 0.24 | 7.2% |
| | MC3 Ether | 1.68 | 56.0% | 1.68 | 57.5% | 1.59 | 56.4% |
| MC3 Ether | Cholesterol | 0.64 | 35.0% | 0.64 | 34.8% | 0.59 | 35.5% |
| EDTA + alpha- | PEG2000-C-DMA | 0.19 | 1.5% | 0.17 | 1.3% | 0.16 | 1.3% |
| tocopherol + | DPPC | 0.29 | 7.6% | 0.23 | 7.0% | 0.23 | 6.8% |
| ascorbyl | MC3 Ether | 1.95 | 58.2% | 1.70 | 58.3% | 1.72 | 58.7% |
| palmitate | | | | | | | |

TABLE 25
MC4 Ether SNALP Lipid Stability by HPLC.

| | | t = 0 | | 2 weeks at RT | | 2 weeks at 40° C. | |
|---|---|---|---|---|---|---|---|
| | | [Lipid] | | [Lipid] | | [Lipid] | |
| Sample | Lipid | (mg/mL) | Mol % | (mg/mL) | Mol % | (mg/mL) | Mol % |
| MC4 Ether | Cholesterol | 0.61 | 33.5% | 0.62 | 34.5% | 0.64 | 38.4% |
| citrate | PEG2000-C-DMA | 0.18 | 1.4% | 0.18 | 1.4% | 0.20 | 1.7% |
| | DPPC | 0.26 | 7.6% | 0.24 | 7.0% | 0.25 | 7.9% |
| | MC4 Ether | 1.69 | 57.6% | 1.67 | 57.1% | 1.40 | 51.9% |
| MC4 Ether | Cholesterol | 0.58 | 33.1% | 0.57 | 33.3% | 0.64 | 33.8% |
| EDTA | PEG2000-C-DMA | 0.17 | 1.4% | 0.17 | 1.4% | 0.18 | 1.4% |
| | DPPC | 0.25 | 7.6% | 0.23 | 6.9% | 0.23 | 6.5% |
| | MC4 Ether | 1.64 | 57.9% | 1.63 | 58.4% | 1.80 | 58.4% |
| MC4 Ether | Cholesterol | 0.61 | 34.1% | 0.57 | 33.5% | 0.61 | 33.6% |
| EDTA + alpha- | PEG2000-C-DMA | 0.18 | 1.4% | 0.17 | 1.4% | 0.19 | 1.4% |
| tocopherol + | DPPC | 0.25 | 7.4% | 0.23 | 7.1% | 0.24 | 6.9% |
| ascorbyl | MC4 Ether | 1.66 | 57.0% | 1.59 | 58.1% | 1.72 | 58.1% |
| palmitate | | | | | | | |

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications, patents, PCT publications, and Genbank Accession Nos., are incorporated herein by reference for all purposes.

1. A method for preventing, decreasing, or inhibiting the degradation of a polyunsaturated cationic lipid present in a nucleic acid-lipid particle, said method comprising:
including an antioxidant in said nucleic acid-lipid particle, wherein said antioxidant comprises ethylenediaminetetraacetic acid (EDTA) or a salt thereof, and
wherein said nucleic acid-lipid particle comprises a nucleic acid, said polyunsaturated cationic lipid, a non-cationic lipid, and a conjugated lipid that inhibits aggregation of said particle.

2. The method of claim 1, wherein said EDTA salt is selected from the group consisting of sodium EDTA, calcium EDTA, calcium disodium EDTA, and mixtures thereof.

3. The method of claim 1, wherein said EDTA or salt thereof is included at a concentration of at least about 20 mM.

4. The method of claim 1, wherein said method further comprises including at least one additional antioxidant in said nucleic acid-lipid particle.

5. The method of claim 4, wherein said at least one additional antioxidant is selected from the group consisting of a primary antioxidant, a secondary antioxidant, salts thereof, and combinations thereof.

6-12. (canceled)

13. The method of claim 1, wherein said polyunsaturated cationic lipid comprises at least one lipid moiety having at least two or at least three sites of unsaturation.

14. The method of claim 13, wherein said at least one lipid moiety is selected from the group consisting of a dodecadienyl moiety, a tetradecadienyl moiety, a hexadecadienyl moiety, an octadecadienyl moiety, an icosadienyl moiety, a dodecatrienyl moiety, a tetradectrienyl moiety, a hexadecatrienyl moiety, an octadecatrienyl moiety, an icosatrienyl moiety, an arachidonyl moiety, a docosahexaenoyl moiety, and combinations thereof.

15. The method of claim 13, wherein said at least one lipid moiety is selected from the group consisting of a linoleyl moiety, a linolenyl moiety, a γ-linolenyl moiety, and combinations thereof.

16-36. (canceled)

37. The method of claim 1, wherein said nucleic acid-lipid particle has a mean diameter of less than about 100 nm after about 1 month at 4° C.

38. The method of claim 1, wherein said nucleic acid-lipid particle has an encapsulation efficiency of greater than about 90% after about 1 month at 4° C.

39. A nucleic acid-lipid particle composition, said composition comprising:
(a) a plurality of nucleic acid-lipid particles comprising: a nucleic acid; a polyunsaturated cationic lipid; a non-cationic lipid; and a conjugated lipid that inhibits aggregation of the particle; and
(b) an antioxidant, wherein said antioxidant comprises EDTA or a salt thereof.

40. The composition of claim 39, wherein said EDTA salt is selected from the group consisting of sodium EDTA, calcium EDTA, calcium disodium EDTA, and mixtures thereof.

41. The composition of claim 39, wherein said composition comprises at least about 20 mM EDTA or a salt thereof.

42. The composition of claim 39, wherein said composition further comprises at least one additional antioxidant in said nucleic acid-lipid particle.

43. The composition of claim 42, wherein said at least one additional antioxidant is selected from the group consisting of a primary antioxidant, a secondary antioxidant, salts thereof, and combinations thereof.

44-50. (canceled)

51. The composition of claim 39, wherein said polyunsaturated cationic lipid comprises at least one lipid moiety having at least two or at least three sites of unsaturation.

52. The composition of claim 51, wherein said at least one lipid moiety is selected from the group consisting of a dodecadienyl moiety, a tetradecadienyl moiety, a hexadecadienyl moiety, an octadecadienyl moiety, an icosadienyl moiety, a dodecatrienyl moiety, a tetradectrienyl moiety, a hexadecatrienyl moiety, an octadecatrienyl moiety, an icosatrienyl moiety, an arachidonyl moiety, a docosahexaenoyl moiety, and combinations thereof.

53. The composition of claim 51, wherein said at least one lipid moiety is selected from the group consisting of a linoleyl moiety, a linolenyl moiety, a γ-linolenyl moiety, and combinations thereof.

54-71. (canceled)

72. The composition of claim 39, wherein said nucleic acid-lipid particle has a mean diameter of less than about 100 nm after about 1 month at 4° C.

73. The composition of claim 39, wherein said nucleic acid-lipid particle has an encapsulation efficiency of greater than about 90% after about 1 month at 4° C.

74. (canceled)

75. A nucleic acid-lipid particle composition, said composition comprising:
(a) a plurality of nucleic acid-lipid particles comprising: a nucleic acid, a polyunsaturated cationic lipid, a non-cationic lipid, and a conjugated lipid that inhibits aggregation of the particle, wherein said polyunsaturated cationic lipid comprises at least one linoleyl moiety, linolenyl moiety, γ-linolenyl moiety, or mixtures thereof; and
(b) an antioxidant, wherein said antioxidant comprises EDTA or a salt thereof.

76-82. (canceled)
